(19) United States
(12) Patent Application Publication
(10) Pub. No.: US 2025/0078994 A1
(43) Pub. Date: Mar. 6, 2025

(54) METHOD OF SURGICAL INSTRUMENT MANAGEMENT AND INFORMATION MANAGEMENT SYSTEM

(71) Applicants: SYSMEX CORPORATION, Kobe-shi (JP); MEDICAROID CORPORATION, Kobe-shi (JP)

(72) Inventors: Masanao OHASHI, Kobe-shi (JP); Masayuki OHIGASHI, Kobe-shi (JP); Kaoru ASANO, Kobe-shi (JP)

(73) Assignees: SYSMEX CORPORATION, Kobe-shi (JP); MEDICAROID CORPORATION, Kobe-shi (JP)

(21) Appl. No.: 18/466,015

(22) Filed: Sep. 13, 2023

(30) Foreign Application Priority Data
Aug. 29, 2023 (JP) 2023- 139182

(51) Int. Cl. G16H 40/40; A61B 34/30; A61B 90/70; G06Q 10/083

(52) U.S. Cl. CPC G16H 40/40; A61B 34/30; A61B 90/70; G06Q 10/083

(57) ABSTRACT

In a method of managing surgical instruments that are used in surgical robotic systems installed in each of facilities, that are collected, for each of which a maintenance comprising at least cleaning and sterilization is performed, and that are shipped to any one of the facilities, the method according to one or more embodiments may include storing, for each of the surgical instruments, status information regarding status of the surgical instruments in association with identification information for each of the surgical instruments, and updating the status information corresponding to a surgical instrument that has been made available for shipping by the maintenance to the status information indicating that the surgical instrument is available to be selected for shipping to any one of the facilities.

### CROSS REFERENCE TO RELATED APPLICATIONS

This application claims priority from prior Japanese Patent Applications No. 2023-139182, filed on Aug. 29, 2023, the entire of which are incorporated herein by reference.

### BACKGROUND

The disclosure relates to a method for managing surgical instruments and an information management system.

In related arts, surgical operations are performed using surgical robotic systems. The surgical robot system is equipped with a console and a surgical robotic arm. During surgery, surgical instruments such as forceps are attached to the surgical robotic arm, and the robotic arm and surgical instruments are operated by an operator operating the console.

Such surgical instruments are maintained by cleaning and sterilization after each surgery, and are used repeatedly. U.S. Patent Application Publication No. 2020/0261173 (patent document 1) describes an automatic processing device for postoperative cleaning processes.

However, compared to conventional surgical instruments used manually by primary surgeons, surgical instruments used in surgical robot systems have fine and complex structures such as wire mechanisms. In addition, the postoperative cleaning process is also complicated, and if not properly maintained, there is a risk of damage to the surgical instruments and residual proteins and other substances adhering to the surgical instruments.

As described above, the method of managing surgical instruments in the related art is burdensome for medical institutions and other facilities that use surgical robot systems, even if the automatic processing device described in the above patent document 1 is used.

### SUMMARY

A method of managing surgical instruments according to one or more embodiments, for example, as shown in FIGS. 1 to 4, 12, 18, 19, 49, 51, 55, 59, is a method of managing surgical instruments (30) that are used in surgical robot systems (11) installed in each of facilities (10), that are collected and for which a maintenance comprising cleaning and sterilization is performed, and that are shipped to any one of the facilities (10), the method may include, storing for each of the surgical instruments (30), status information regarding status of the surgical instruments (30) in association with identification information for each of the surgical instruments (30), and updating the status information corresponding to a surgical instrument (30) that has been made available for shipping by the maintenance (step S212 in FIG. 18). to the status information indicating that the surgical instrument is available to be selected for shipping to any one of the facilities (10).

According to the method for managing surgical instruments according to one or more embodiments, it may be possible to reduce the burden on facilities using the surgical robot system because maintenance such as cleaning and sterilization of surgical instruments used in the surgical robot system need not be performed at the facility where the surgical robot system is installed. In addition, since it may be possible to manage whether or not the surgical instruments may be selected for shipping to a facility based on the status information of the surgical instruments, surgical instruments that become available for shipping through maintenance may be identified from among multiple surgical instruments and provided to a facility and may be reused.

An information management system for one or more embodiments is, for example, as shown in FIGS. 1 to 4, 10, 12, 18, 19, 49, 51, 55, 59, a system to manage information about surgical instruments that are used in a surgical robot system (11) installed in each of the facilities (10), that are collected and for each of which a maintenance comprising cleaning and sterilization is performed, and that are shipped to any one of the facilities (10). An information management system (40) for managing surgical instruments (30) may include: a memory (152) storing, for each of the surgical instruments (30), status information on status of the surgical instruments (30) in association with identification information for each of the surgical instruments (30), a controller (141) which updates the status information corresponding to a surgical instruments (30) that has become deliverable through maintenance to the status information indicating that it may be selected for shipping to the facility (10).

According to the information management system according to one or more embodiments, the maintenance of surgical instruments used by the surgical robot system, such as cleaning and sterilization, does not need to be performed at the facility where the surgical robot system is installed, which may reduce the burden on the facility using the surgical robot system. In addition, since the system may manage whether or not the surgical instruments may be selected for shipping to a facility based on the status information of the surgical instruments, surgical instruments that become available for shipping due to maintenance may be identified from among multiple surgical instruments and provided to a facility, and may be reused.

### BRIEF DESCRIPTION OF DRAWINGS

FIG. 1 is a schematic diagram illustrating a reuse of surgical instruments in accordance with embodiment 1.

FIG. 2 is a block diagram illustrating a functional structure of a surgical instrument management system according to embodiment 1.

FIG. 3 is a schematic diagram of a process flow in the facility according to embodiment 1.

FIG. 4 is a schematic diagram illustrating a flow of processing within a device center according to embodiment 1.

FIG. 5 is a block diagram illustrating a functional configuration of a surgical robot system and surgical instruments according to embodiment 1.

FIG. 6 is a diagram illustrating an appearance of a patient-side device and surgical instruments according to embodiment 1.

FIG. 7 is a diagram illustrating a configuration of a surgical instrument in accordance with embodiment 1.

FIG. 8 is a diagram illustrating appearance of an operating system according to embodiment 1.

FIG. 9 is a block diagram illustrating functional configuration of a controller according to embodiment 1.

FIG. 10 is a block diagram illustrating a functional structure of an information management system according to embodiment 1.

FIG. 11 is a block diagram illustrating a functional configuration of a work terminal located at a facility and at a device center according to embodiment 1.

FIG. 12 is a schematic diagram illustrating details of a structure of a surgical instrument table and status IDs stored in a database, according to embodiment 1.

FIG. 13 is a schematic diagram illustrating a composition of a demand information table stored in a database according to embodiment 1.

FIG. 14 is a schematic diagram illustrating a structure of a shipping information table stored in a database, according to embodiment 1.

FIG. 15 is a flowchart illustrating a process of transmitting an operation log by a controller according to embodiment 1.

FIG. 16 is a schematic diagram illustrating a configuration of an operation log for an arm to which a surgical instrument is attached, according to embodiment 1.

FIG. 17 is a flowchart illustrating a process of updating status to collection by an information management system according to embodiment 1.

FIG. 18 is a flowchart illustrating a processing of a work terminal and information management system in a maintenance process of a device center, according to embodiment 1.

FIG. 19 is a schematic diagram illustrating an update screen according to embodiment 1.

FIG. 20 is a flowchart illustrating a work of an operator in a maintenance process of a device center, according to embodiment 1.

FIG. 21 is a flowchart illustrating a work terminal and information management system processing in a device center selection process according to embodiment 1.

FIG. 22 is a schematic diagram illustrating a shipping decision screen displayed on a display of a work terminal in a device center according to embodiment 1.

FIG. 23 is a schematic diagram illustrating an example of a modified shipping decision screen according to embodiment 1.

FIG. 24 is a flowchart illustrating a processing of a work terminal and information management system in a shipping process of a device center, according to embodiment 1.

FIG. 25 is a schematic diagram illustrating a shipping screen displayed on a display of a work terminal in a device center according to embodiment 1.

FIG. 26 is a flowchart illustrating a processing of a work terminal and information management system during inventory management at a device center, according to embodiment 1.

FIG. 27 is a schematic diagram illustrating an inventory screen displayed on a display of a work terminal in a device center according to embodiment 1.

FIG. 28 is a flowchart illustrating a processing of a work terminal and information management system in a device center selection process according to embodiment 1 in a Modification 1.

FIG. 29 is a schematic diagram illustrating a shipping decision screen displayed on a display of a work terminal in a device center according to embodiment 1 in modification 1.

FIG. 30 is a schematic diagram illustrating a details of a status ID according to embodiment 1 in modification example 2.

FIG. 31 is a schematic diagram illustrating a structure of a surgery schedule table and the quantity of surgical instruments table stored in a database according to embodiment 2.

FIG. 32 is a schematic diagram illustrating a composition of a demand information table stored in a database according to embodiment 2.

FIG. 33 is a schematic diagram illustrating shipment confirmation at a device center according to embodiment 2.

FIG. 34 is a flowchart illustrating a processing of a facility's work terminal and information management system when entering a surgical schedule, according to embodiment 2.

FIG. 35 is a schematic diagram illustrating a structure of a surgery schedule entry screen displayed on a display of a work terminal in a facility, in accordance with embodiment 2.

FIG. 36 is a schematic diagram illustrating a shipping decision screen displayed on a display of a work terminal in a device center according to embodiment 2.

FIG. 37 is a schematic diagram illustrating an inventory screen displayed on a display of a work terminal in a device center according to embodiment 2.

FIG. 38 is a schematic diagram illustrating a structure of a surgery schedule table stored in a database according to embodiment 3.

FIG. 39 is a schematic diagram illustrating a structure of a combination table stored in a database according to embodiment 3.

FIG. 40 is a schematic diagram illustrating a structure of a surgery schedule entry screen displayed on a display of a work terminal in a facility, in accordance with embodiment 3.

FIG. 41 is a schematic diagram illustrating a structure of a surgery schedule table according to embodiment 3 in a modification example.

FIG. 42 is a schematic diagram illustrating a structure of a combination table according to embodiment 3 in a modified example.

FIG. 43 is a schematic diagram illustrating a structure of a utilization result table according to embodiment 4.

FIG. 44 is a flowchart illustrating a processing of an information management system and work terminal when registering demand information, according to embodiment 4.

FIG. 45 is a schematic diagram illustrating a structure of a utilization result table according to embodiment 5.

FIG. 46 is a flowchart illustrating a processing of an information management system and work terminal when updating combination information, according to embodiment 5.

FIG. 47 is a diagram illustrating a lead time set for each status ID according to embodiment 6.

FIG. 48 is a schematic diagram illustrating a shipping decision screen displayed on a display of a work terminal in a device center, in accordance with embodiment 6.

FIG. 49 is a schematic diagram illustrating a details of a status ID according to embodiment 7.

FIG. 50 is a schematic diagram illustrating a structure of a surgical instrument table stored in a database according to embodiment 7.

FIG. 51 is a schematic diagram illustrating a device center maintenance process flow according to embodiment 7.

FIG. 52 is a schematic diagram illustrating a configuration of a screen for inspection displayed on a display of a work terminal in an inspection area, according to embodiment 7.

FIG. 53 is a schematic diagram illustrating a configuration of a confirmation screen displayed on a display of a work terminal in an inspection area, according to embodiment 7.

FIG. 54 is a schematic diagram illustrating a structure of a surgical instrument table stored in a database according to embodiment 7 in modification 1.

FIG. 55 is a block diagram illustrating a functional structure of a surgical instrument management system according to embodiment 7 in modification 2.

FIG. 56 is a flowchart illustrating a process of inspection using a surgical robot system according to embodiment 7 in modification 2.

FIG. 57 is a diagram illustrating a usage status ID added to a status ID according to embodiment 8.

FIG. 58 is a diagram illustrating a refurbish ID added to a status ID according to embodiment 8 in a modified example.

FIG. 60 is a schematic diagram illustrating a structure of a facility table and device center table stored in a database DB according to embodiment 9.

FIG. 61 schematically shows the structure of the surgical instrument table stored in the database DB, according to embodiment 9.

FIG. 62 is a schematic diagram illustrating a shipping decision screen displayed on a display of a work terminal in a device center, according to embodiment 9.

FIG. 63 is a schematic diagram illustrating a shipping decision screen displayed on a display of a work terminal in a device center, according to embodiment 10.

FIG. 64 is a schematic block diagram illustrating a structure of a surgical instrument table stored in a database, according to embodiment 11.

FIG. 65 is a schematic diagram illustrating a shipping decision screen displayed on a display of a work terminal in a device center, according to embodiment 11.

### DETAILED DESCRIPTION

### Embodiment 1

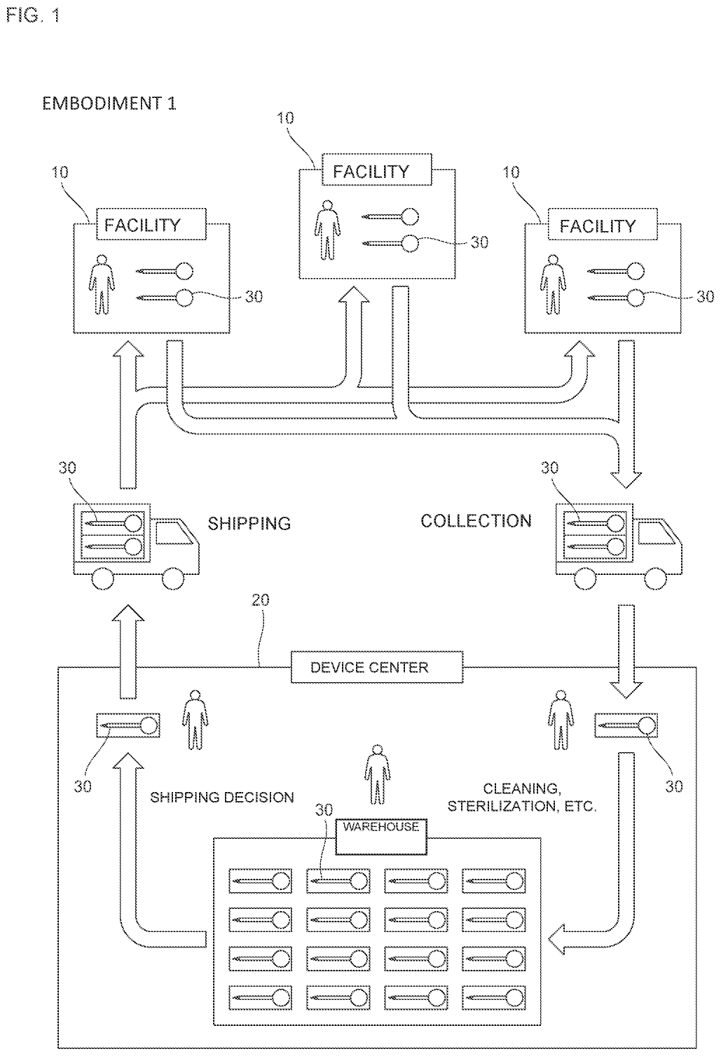

FIG. 1 is a schematic diagram illustrating the reuse of surgical instruments 30.

A surgical robotic system 11, described below, is installed in a medical facility or other facility 10. During surgery, surgical instruments 30 are attached to the surgical robot system 11. The surgical robotic system 11 is operated by an operator, who is a physician, to perform surgery on the patient. When the surgery is completed, the surgical instruments 30 used at each facility 10 are collected at the device center 20.

The device center 20 collects surgical instruments 30, performs maintenance such as cleaning and sterilization on the collected surgical instruments 30, and stores the maintained surgical instruments 30 in a warehouse. When a demand for surgical instruments 30 arises from the facilities 10, the surgical instruments 30 in the warehouse in the device center 20 are targeted for shipping according to the demand and are delivered to each facility 10. At the facility 10, surgery is performed by the surgical robot system 11 using the surgical instruments 30 delivered from the device center 20.

When the maintenance of surgical instruments 30 is performed at the device center 20 as shown in FIG. 1, the burden of maintaining surgical instruments 30 at each facility 10 is reduced. In addition, when the surgical instruments 30 are reused, the use of the surgical instruments 30 can be achieved more environmentally friendly.

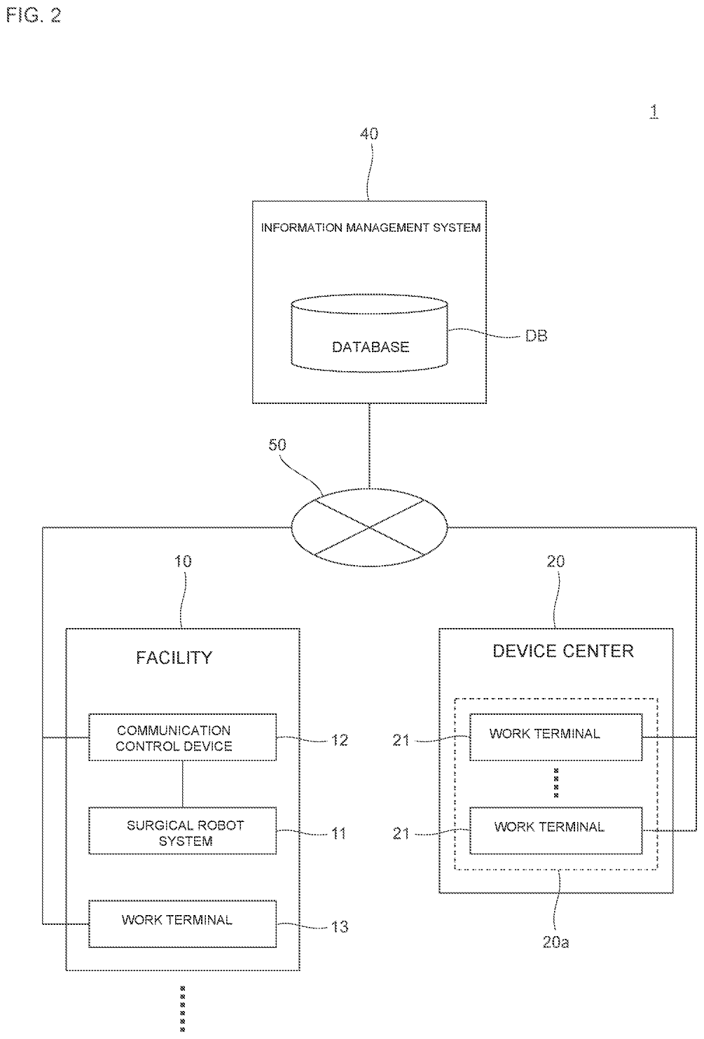

FIG. 2 is a block diagram showing the functional structure of the surgical instrument management system 1.

The surgical instrument management system 1 includes a surgical robot system 11, a communication control device 12, a work terminal 13, a work terminal 21, and an information management system 40. The communication control device 12 and the information management system 40, the work terminal 13 and the information management system 40, and the work terminal 21 and the information management system 40 are connected by a network 50. The network 50 includes, for example, the Internet. The communication control device 12 includes, for example, a computer.

Each of the multiple facilities 10 includes a surgical robot system 11, a communication control device 12, and a work terminal 13. The surgical robot system 11 is communicatively connected to the information management system 40 via the communication control device 12 and the network 50. The work terminal 13 is communicatively connected to the information management system 40 via the network 50. A maintenance management system 20A including a plurality of work terminals 21 is installed in the device center 20. The plurality of work terminals 21 are communicably connected to the information management system 40 via the network 50.

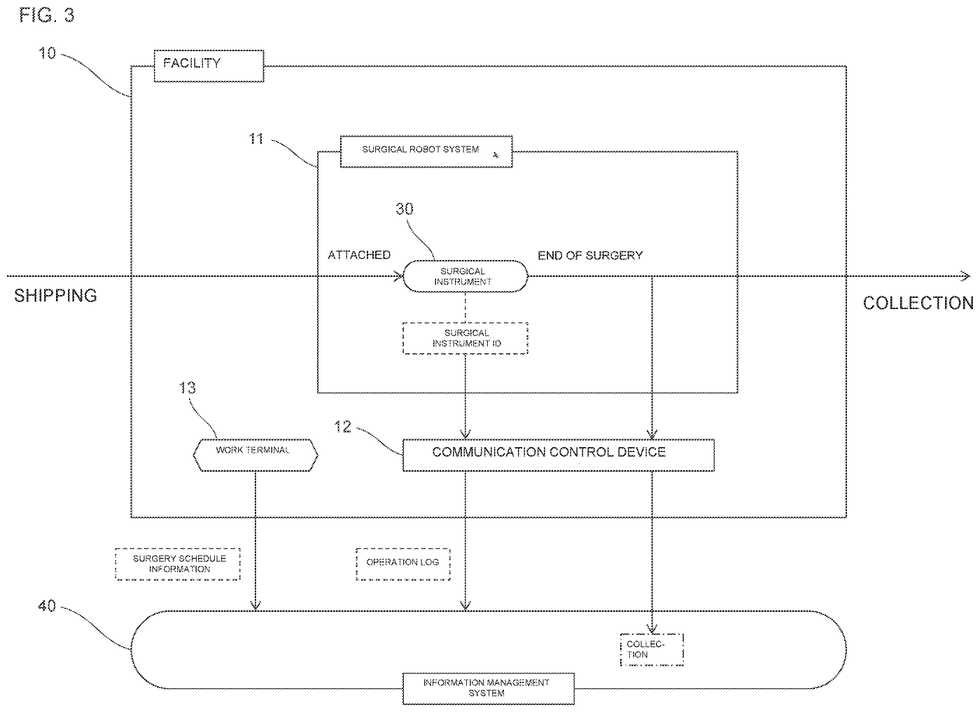

FIG. 3 is a schematic diagram of the process flow in the facility 10.

At facility 10, prior to the date of the surgery, the operator enters scheduled surgery information, for example, as demand information, into work terminal 13. The surgery schedule information includes, for example, the type and quantity of surgical instruments 30 required for the surgery. The surgery schedule information entered into the work terminal 13 is transmitted to the information management system 40. The surgical instruments 30 required for the surgery are then delivered to the facility 10 prior to the date the surgery is to be performed.

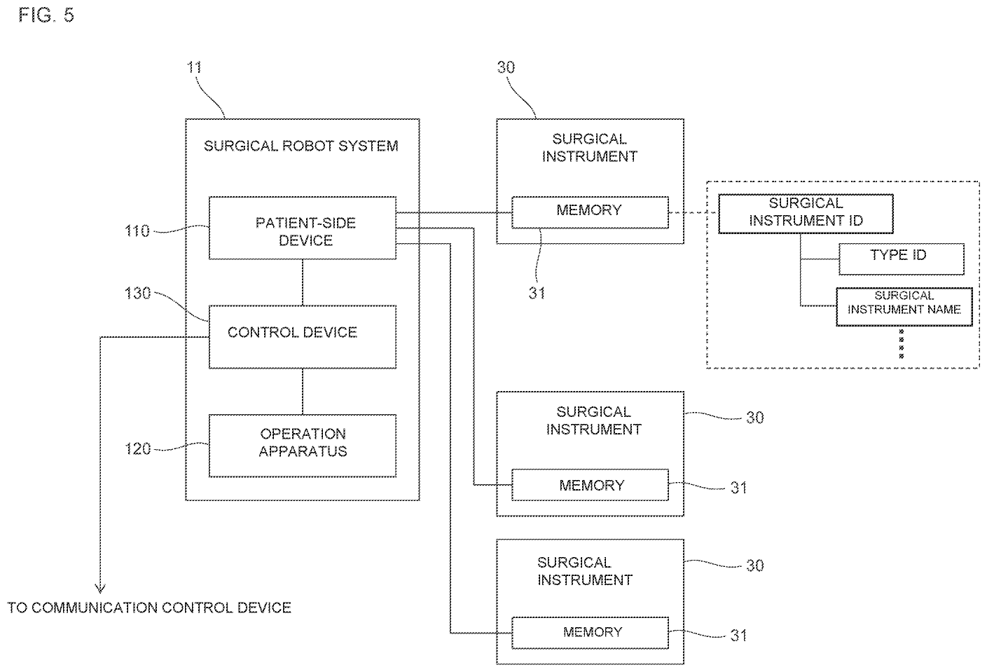

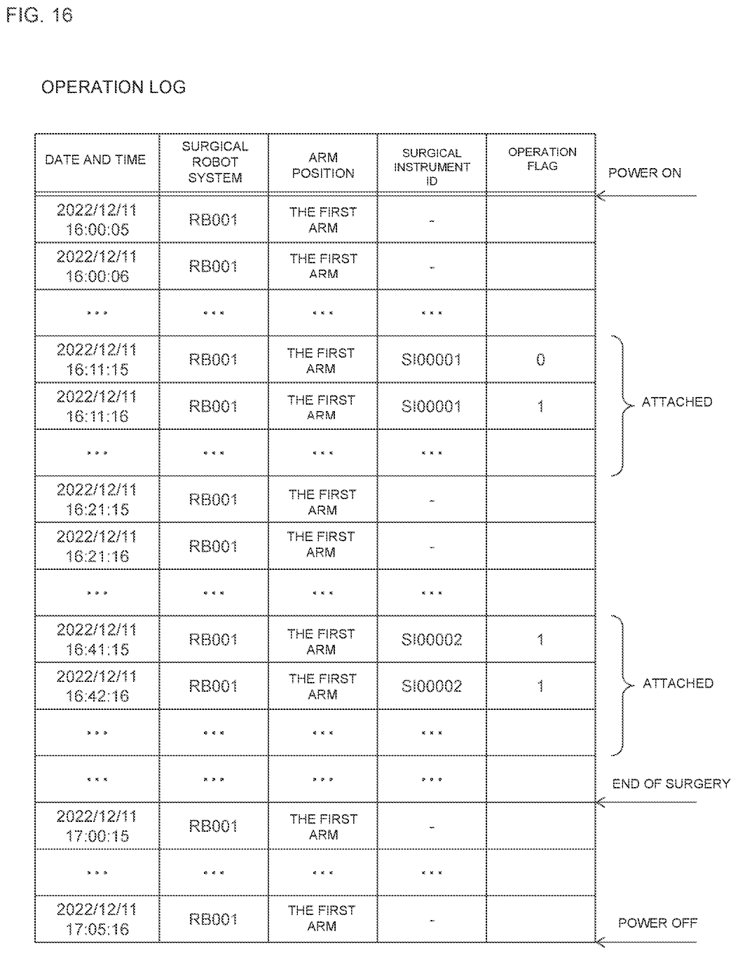

The surgical instruments 30 delivered to the facility 10 are attached to the surgical robot system 11 during surgery. The memory 31 (see FIG. 5) of the surgical instrument 30 stores a surgical instrument ID that can identify the surgical instrument 30. The surgical instrument ID of the surgical instrument 30 attached to the surgical robot system 11 is transmitted to the information management system 40 via the communication control device 12 as part of the operation log of the surgical robot system 11. The operation log is described later with reference to FIG. 16.

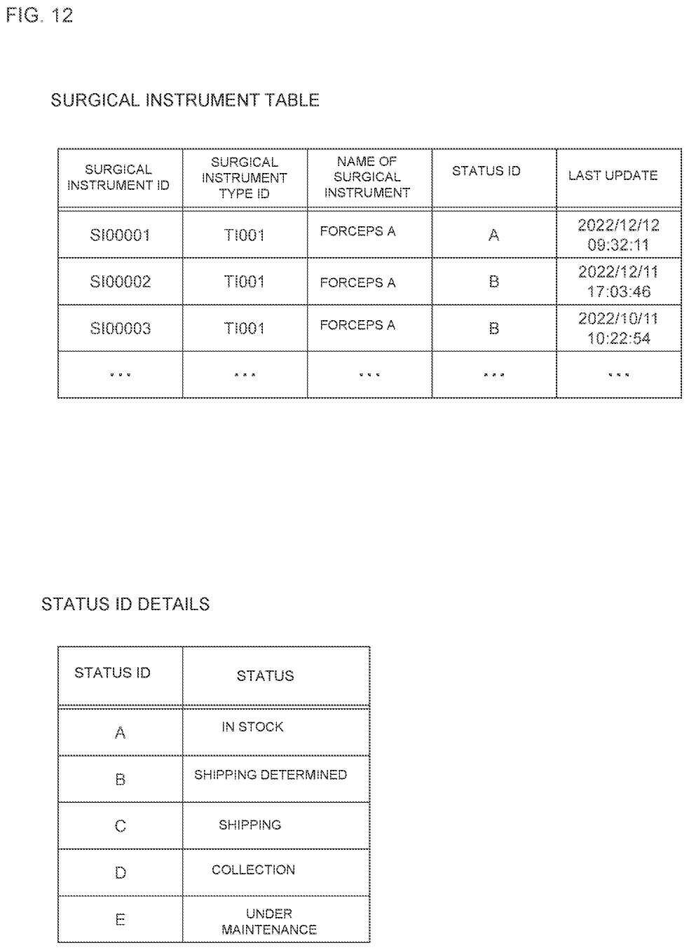

When the surgery by the surgical robot system 11 is completed, information indicating the completion of the surgery is sent to the information management system 40 via the communication control device 12. The information management system 40 then sets the status of the surgical instruments 30 used in this surgery to “collect”. The status of the surgical instruments 30 will be explained later with reference to FIG. 12. The used surgical instruments 30 are then collected to the device center 20.

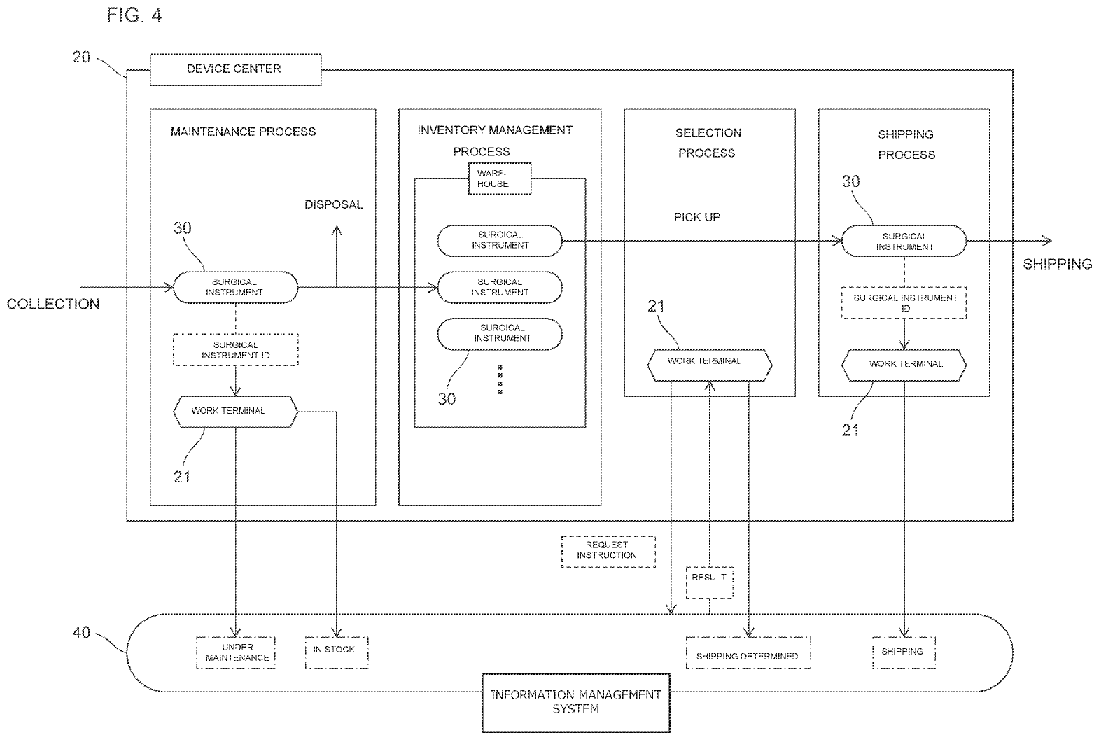

FIG. 4 is a schematic diagram of the process flow within the device center 20.

The processing process performed at the device center 20 includes a maintenance process, an inventory management process, a selection process, and a shipping process.

In the maintenance process, the operator enters the surgical instrument ID of the surgical instrument 30 collected from the facility 10 into the work terminal 21. The surgical instrument ID entered into the work terminal 21 is transmitted to the information management system 40, and the status of the surgical instrument 30 stored in the information management system 40 is set to “under maintenance”. Thereafter, maintenance such as cleaning and sterilization is performed on the surgical instruments 30, and the surgical instruments 30 are sealed and packaged in a tray or other container. The operator enters the surgical instrument ID of the surgical instrument 30 for which maintenance has been completed into the work terminal 21. The surgical instrument ID entered into the work terminal 21 is transmitted to the information management system 40, and the status of the surgical instrument 30 stored in the information management system 40 is set to “In stock”. The surgical instruments 30 then proceed to the inventory management process.

In the maintenance process, for example, if it is determined that the quantity of times the surgical instruments 30 have been used exceeds a predetermined threshold, or if it is determined that the surgical instruments 30 are malfunctioning, the surgical instruments 30 are discarded and new surgical instruments 30 are replenished. If the frequency of surgeries at each of the facilities 10 increase, or if new facilities 10 are added, the total quantity of surgical instruments 30 must be increased, and new surgical instruments 30 are replenished. The replenished surgical instruments 30 proceed through the maintenance process to the inventory management process.

In the inventory management process, surgical instruments 30 that have been packaged through the maintenance process are stored in a warehouse.

In the selection process, the operator operates the work terminal 21 to send instructions to the information management system 40 to request demand information and surgical instrument information, and receives the resulting demand information and surgical instrument information from the information management system 40. The demand and surgical instrument information includes the type and quantity of surgical instruments 30 required at the facility 10 and a list of the surgical instruments 30 stored as inventory in the warehouse of the device center 20.

The operator operates the work terminal 21, refers to the demand information and surgical instrument information, and determines the surgical instruments 30 to be delivered. As a result, the status of the surgical instruments 30 determined as the shipping target is set to “shipping determined” in the information management system 40, and the surgical instruments 30 are associated with the shipping destination facility 10. The operator then picks up the surgical instruments 30 that have been set to shipping determined from the warehouse.

In the shipping process, the operator performs shipping procedures for the surgical instruments 30 picked up from the warehouse, and enters the surgical instrument ID of the surgical instruments 30 for which shipping procedures have been performed into the work terminal 21. The surgical instrument ID entered into the work terminal 21 is transmitted to the information management system 40, and in the information management system 40, the status of the surgical instrument 30 for which the shipping procedure has been performed is set to “shipping” and stored in association with the surgical instrument 30 and the shipping destination facility 10. The operator then delivers the surgical instruments 30 to the destination facility 10.

FIG. 5 is a block diagram showing the functional configuration of the surgical robot system 11 and surgical instruments 30.

The surgical robot system 11 is a device used in endoscopic surgery. The surgical robot system 11 has a surgical robot 110 (also called a patient-side device), an operation apparatus 120, and a control device 130 that controls the patient-side device 110 and the operation apparatus 120. The control device 130 is built into the patient-side device 110 and is communicatively connected to the communication control device 12. The control device 130 may be built into the operation apparatus 120 or may be located independently of the patient-side device 110 and the operation apparatus 120. Three surgical instruments 30 are attached to the patient-side device 110, and surgery is performed on the patient by the operation of the three surgical instruments 30. The quantity of surgical instruments 30 attached to the patient-side device 110 is not limited to three, but may be one, two, or four or more.

The surgical instrument 30 has a memory 31. In FIG. 5, the information stored in memory 31 is shown within a dashed line enclosure. The memory 31 stores the surgical instrument ID, the type ID, the name of the surgical instrument, and other information about the surgical instrument 30. The memory 31 may further include the maximum number of usage, current number of usage, lot number, manufacturer, and other information regarding the surgical instrument 30 in question.

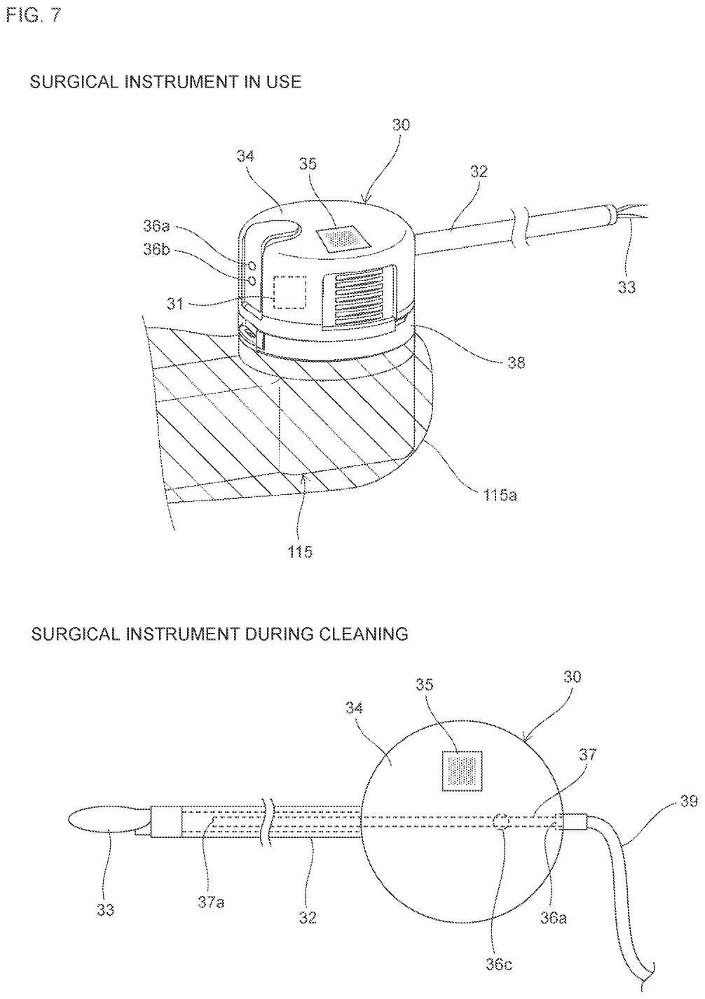

The surgical instrument ID includes an ID that can individually identify surgical instruments 30. The surgical instrument ID includes, for example, a serial number. The information stored in memory 31 is associated with each other based on the surgical instrument ID. The type ID includes an ID that can individually identify the type of the surgical instruments 30 in question. The surgical instrument name includes the name of the surgical instrument 30 indicated by the type ID. The surgical instrument name indicates the function of the relevant surgical instrument 30 and the type of end effector 33 (see FIG. 7). The surgical instrument name includes, for example, forceps A, forceps B, forceps C, high frequency knife A, high frequency knife B, high frequency knife C, etc.

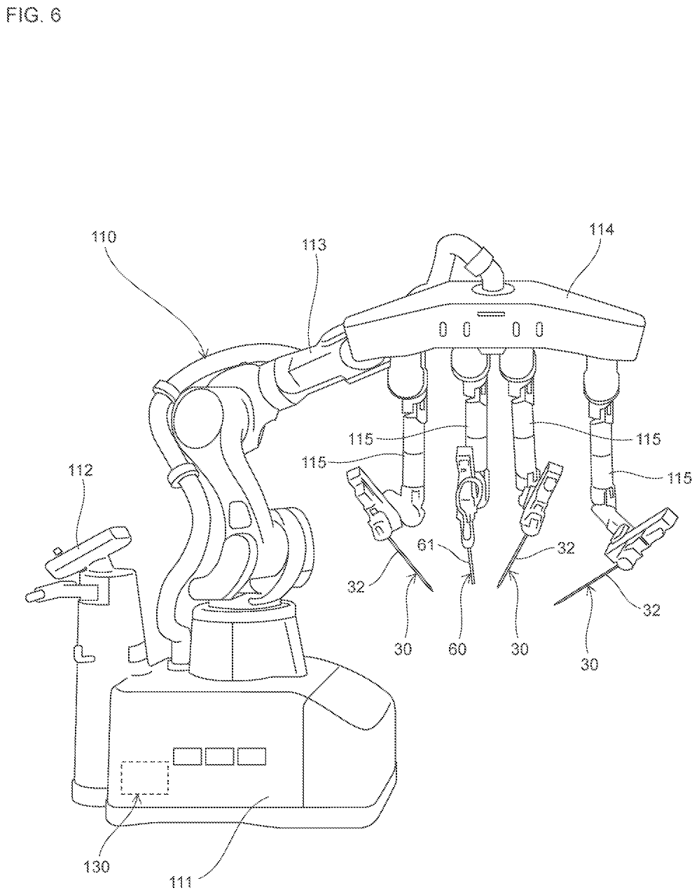

FIG. 6 shows a diagram of the patient-side device 110 and the surgical instrument 30.

The patient-side device 110 includes a patient-side device with four robotic arms (hereinafter referred to as arms) 115 that can be equipped with three surgical instruments 30 and one endoscope 60. The patient-side device 110 operates according to commands transmitted from the control device 130 when the operation apparatus 120 is operated by an operator, a physician.

The patient-side device 110 includes a base 111, an operation unit 112, a base arm 113, a support 114, and four arms 115. three surgical instruments 30 and one endoscope 60 are removably attached to the four arms 115, respectively.

The operation unit 112 is located on the base 111 and includes a display and an input unit. While viewing the display of the operation unit 112, the operator operates the input section of the operation unit 112 to move the support 114 by driving the base arm 113 of the patient-side device 110 to position the arm 115 in a position suitable for preparing for surgery. The base arm 113 is installed at its rear end on the base 111 and includes a robotic arm with multiple joints. The support 114 has its top surface connected to the tip of the base arm 113 in a pivoting manner, and moves with the movement of the tip of the base arm 113 and pivots around the axis of the tip of the base arm 113.

Four arms 115 have their upper ends installed on the underside of the support 114; each of the four arms 115 includes an arm with twelve axes and inter-axial joints. At the lower end of the arm 115 is a support configured to attach and detach a surgical instrument 30 and an endoscope 60 via an adapter 38 (see FIG. 7), and the surgical instrument 30 or endoscope 60 is attached to the support via the adapter 38. The surgical instrument 30 has an elongated shaft 32 and the endoscope 60 has an elongated shaft 61. Endoscope 60 includes, for example, a 3D endoscope.

Prior to the start of surgery, each arm 115 (see FIG. 7) is positioned above the patient lying on the operating table, with drape 115a and adapter 38 attached to the support fixture. A guide tube (trocar) is inserted into the abdomen of the patient lying on the operating table, and the surgical instruments 30 and the shafts 32 and 61 of the endoscope 60, attached to each arm 115 via the adapter 38, are inserted into the patient through the guide tube. Thus, preparations for surgery are made. Usually, the surgical instruments 30 are replaced multiple times during surgery with different types of surgical instruments 30 depending on the nature of the procedure.

FIG. 7 shows the configuration of the surgical instrument 30. The upper part of FIG. 7 is a diagrammatic view of the configuration of the surgical instrument 30 attached to the end of the arm 115, and the lower part of FIG. 7 is a schematic plan view of the configuration of the surgical instrument 30 during cleaning.

As shown in the upper and lower sections of FIG. 7, surgical instrument 30 has a memory 31, shaft 32, end effector 33, housing tag 34, tag 35, cleaning liquid supply ports 36a, 36b, 36c, tube 37, and adapter 38. The surgical instruments 30 include various types of instruments, such as grasping forceps, scissors, hooks, high-frequency knives, bipolar, monopolar, snare wires, clamps, and staplers. The 33 end-effectors depend on the type of surgical instrument 30. The top and bottom rows of FIG. 7 show the end effector 33 when the surgical instruments 30 are scissors and grasping forceps, respectively, as an example.

Shaft 32 has an elongated shape. End effector 33 is located at the end of shaft 32. The housing tag 34 has a substantially cylindrical shape and supports the end of the shaft 32 opposite the end effector 33. The memory 31 is located inside the housing tag 34, in an enclosure that prevents the entry of cleaning liquid, and is composed, for example, of a flash memory. Tag 35 is affixed to the top surface of housing tag 34, and the surgical instrument ID of the surgical instrument 30 is printed on the surface of tag 35, for example, as a bar code or QR code (registered trademark). The string of the surgical instrument ID is also printed on the surface of the tag 35.

The cleaning liquid supply ports 36a, 36b, and 36c are holes for supplying cleaning water to the inside of the shaft 32 and housing tag 34 when cleaning the surgical instrument 30. The cleaning liquid supply ports 36a and 36b are located on the sides of the housing tag 34, and the cleaning liquid supply port 36c is located on the underside of the housing tag 34. The tube 37 is arranged inside the housing tag 34 and shaft 32 while extending from the cleaning liquid supply port 36a to near the end of the shaft 32.

The drape 115a is removably installed on the arm 115 to cover the arm 115 and is a housing to prevent infection to the patient caused by the arm 115. Adapter 38 is attached to the support of the arm 115 with the drape 115a and secures the drape 115a. When the surgical instrument 30 is connected to the arm 115, the adapter 38 is attached to a support on the arm 115 side. This allows each of the surgical instruments 30 and the arm 115 to be mechanically and electrically connected via the adapter 38. The electrical connection between each of the surgical instruments 30 and the arm 115 allows the patient-side device 110 to read and write information to and from the memory 31 of the surgical instrument 30. To remove the surgical instrument 30 from the arm 115, the surgical instrument 30 is removed from the adapter 38, which is attached to a support on the side of the arm 115.

When surgical instruments 30 are cleaned in the device center 20, a hose 39 is connected to the cleaning liquid supply port 36a and cleaning water is supplied into the tube 37 via the hose 39, as shown in the lower part of FIG. 7. The cleaning water supplied into the tube 37 is supplied from the tip 37a of the tube 37 into the shaft 32 and flows through the shaft 32 into the housing tag 34. A hose 39 is connected to the cleaning liquid supply port 36b, and the cleaning liquid is supplied into the housing tag 34 through the hose 39. The cleaning liquid supplied inside the housing tag 34 flows out through the gaps in the housing tag 34 and through the cleaning liquid supply port 36c.

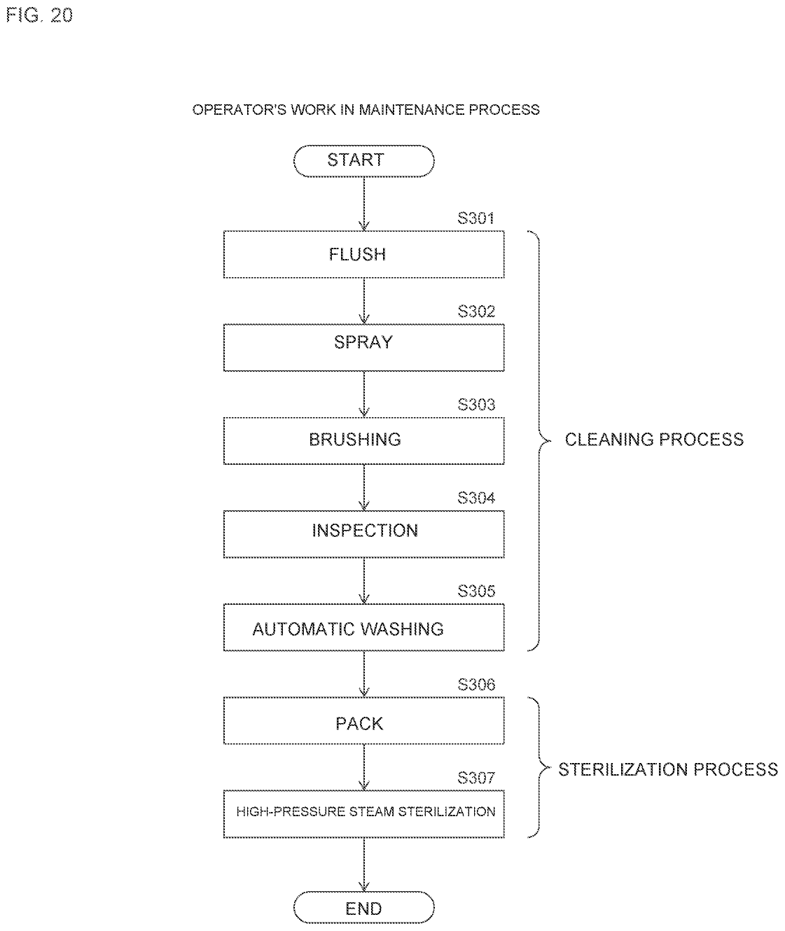

The process in which cleaning liquid is supplied to the cleaning liquid supply ports 36a and 36b in the cleaning of the surgical instruments 30 is referred to as flushing. The process of cleaning the surgical instruments 30 consists of five steps, including the flush. The cleaning process of the surgical instruments 30 will be explained later with reference to FIG. 20.

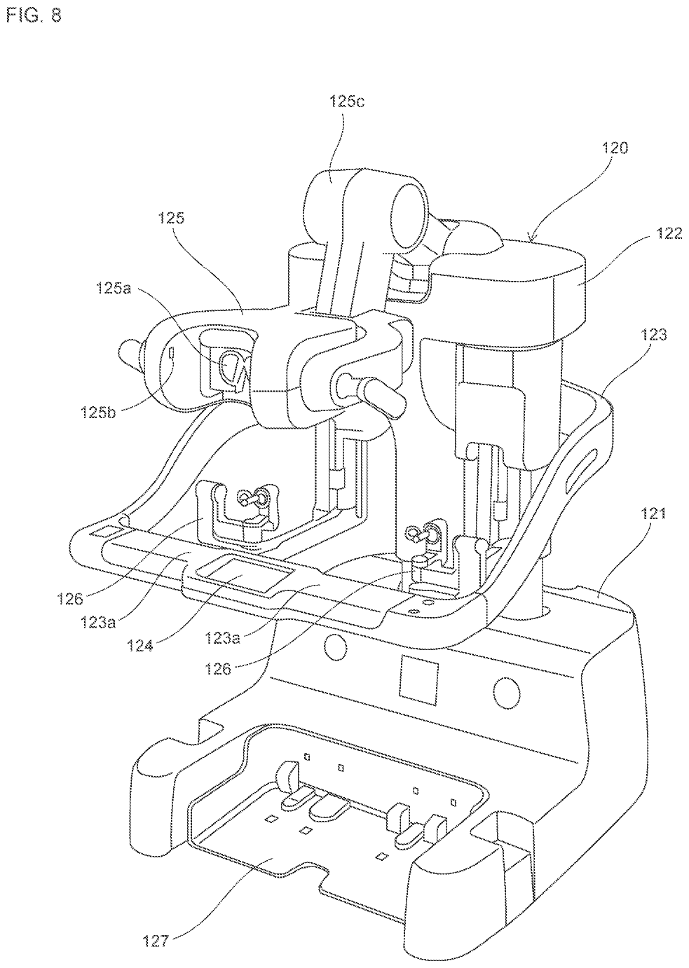

FIG. 8 is a diagram of the appearance of the operation apparatus 120.

The operation apparatus 120 is an operator-side device for driving the patient-side device 110 by being operated by the operator, a physician, during endoscopic surgery.

The operation apparatus 120 includes a base 121, a support 122, a frame member 123, an operation panel 124, a viewer unit 125, two hand controllers 126, and a foot unit 127.

The support 122 is installed on top of the base 121 so that it can be moved up and down. A frame member 123 is installed on the support 122. An operation panel 124 is installed on the top surface of the front center of the frame member 123. Armrests 123a are formed on the left and right sides of the operation panel 124 for the operator to place the operator's elbows on when operating the hand controller 126. The height of the arm rests 123a can be changed by moving the support 122 up and down with respect to the base 121.

The viewer unit 125 is supported by the end of an arm 125c installed on the top surface of the support 122, and the height of the arm 125c can be changed by rotating the joint of the arm 125c. The viewer unit 125 has a viewer 125a and a head sensor 125b. The viewer 125a includes a display for displaying images taken by the endoscope 60 (endoscopic images) and various screens. The head sensor 125b is provided across the area in front of the viewer 125a and includes a transmitted light photoelectric sensor. When an operator looks into the viewer 125a, the operator's head approaches the viewer 125a, shading the area between the head sensors 125b and detecting that the operator is looking at the viewer 125a.

Two hand controllers 126, each with seven axes and inter-axial joints, are installed on a support 122. The left and right hand controllers 126 include input devices for the operator to manipulate the four arms 115 of the patient-side device 110 using the left and right hands, respectively.

When the operator inputs the operation of movement to the hand controller 126, each joint of the hand controller 126 rotates on its axis. This drives the target arm 115, and the surgical instrument 30 or endoscope 60 attached to the arm 115 moves. When the surgical instrument 30 with forceps as the end effector 33 is attached to the target arm 115, when the operator inputs a grasping operation to the hand controller 126, the tip of the forceps is driven to open and close.

The foot unit 127 is located on the lower front side of the base 121. The foot unit 127 includes an input device for the operator to operate the patient-side device 110 using the left and right feet.

By performing operations on the foot unit 127, the operator can switch between the surgical instruments 30 and endoscopes 60 to be operated by the left and right hand controllers 126. The operator can also cause the end effector 33 on the tip of the surgical instruments 30 to perform a predetermined operation (e.g., incision, coagulation, etc.) by performing an operation on the foot unit 127.

Before the start of the surgery, the operator sits in a chair, places both arms on the armrests 123a and both feet inside or in front of the foot unit 127. The operator places the operator's head inside the viewer unit 125 and looks into the viewer 125a. When the head sensor 125b detects that the operator's head is positioned inside the viewer unit 125, the patient-side device 110 can be operated by the operation apparatus 120.

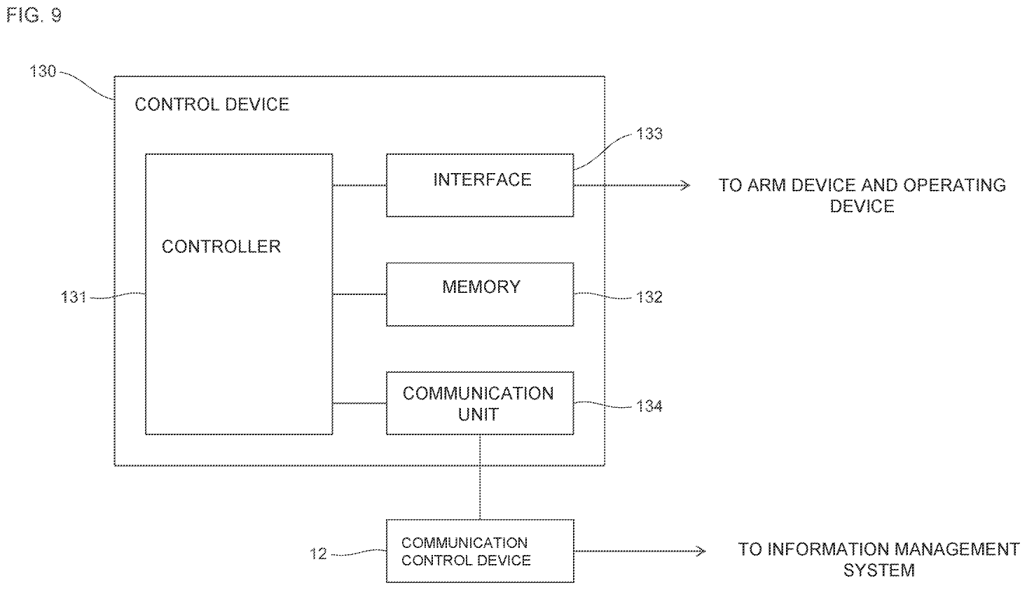

FIG. 9 is a block diagram showing the functional structure of the control device 130.

The controller 130 has a controller 131, a memory 132, an interface 133, and a communication section 134.

The controller 131 includes, for example, a CPU. The controller 131 executes a computer program stored in the memory 132 to control the hardware of the control device 130 and to execute various processes. The memory 132 includes, for example, an SSD or HDD. The interface 133 includes a communication interface capable of communicating with the patient-side device 110 and the operation apparatus 120 based on a predetermined communication standard. The communication section 134 includes, for example, a communication interface capable of communicating with the communication control device 12 based on an Ethernet standard.

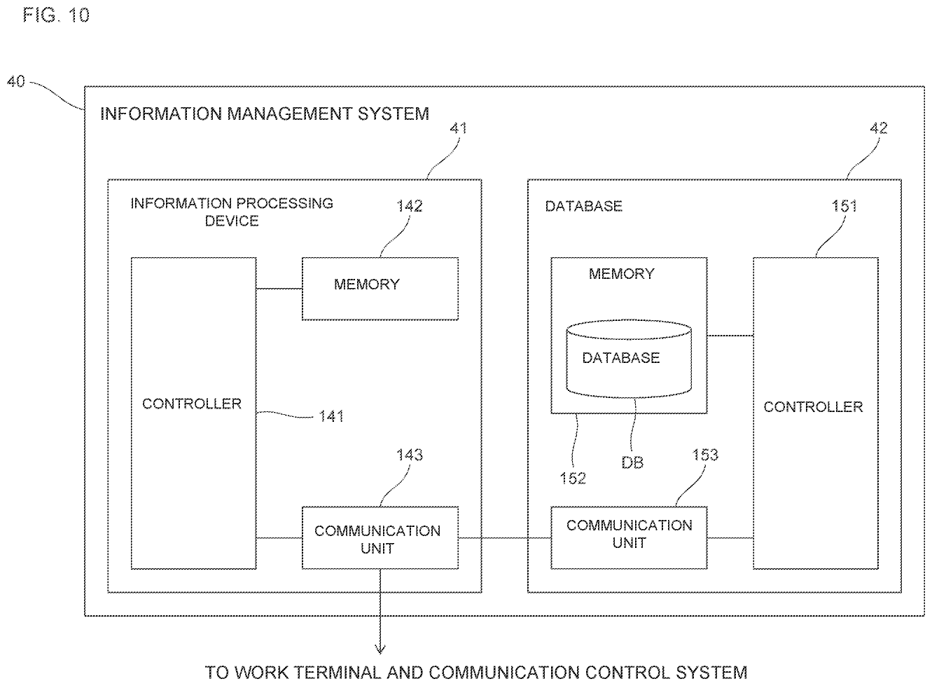

FIG. 10 is a block diagram showing the functional structure of the information management system 40.

The information management system 40 includes an information processing device 41 and a database device 42. The information management system 40 writes information received from the communication control device 12 and work terminals 13 and 21 into the database DB, and retrieves information stored in the database DB and transmits it to the communication control device 12 and work terminals 13 and 21.

The information processing device 41 has a controller 141, a memory 142, and a communication section 143. The controller 141 includes, for example, a CPU. The controller 141 controls each hardware section of the information processing device 41 and executes various processes by executing a computer program stored in the memory 142. The controller 141 stores information in the database DB and retrieves information from the database DB by sending instructions to the database device 42. The memory 142 includes, for example, SSD and HDD. The communication section 143 includes an interface capable of communicating with the work terminals 13, 21, the communication control device 12 and the database device 42 based on an Ethernet standard, for example.

The database device 42 has a controller 151, a memory 152, and a communication unit 153. The controller 151 includes, for example, a CPU. The controller 151 controls each hardware section of the database device 42 and executes various processes by executing a computer program stored in the memory 152. The controller 151 stores information in the database DB and retrieves information from the database DB in response to instructions from the controller 141 of the information processing device 41. The memory 152 includes, for example, SSD and HDD. The memory 152 stores the database DB. The database DB stores various tables and operation logs of the surgical instruments 30 transmitted from the surgical robot system 11. The communication unit 153 includes, for example, an interface that can communicate with the information processing device 41 based on the Ethernet standard.

Although the information management system 40 was configured with an information processing device 41 and a database device 42, the configuration of the information management system 40 is not limited to this. For example, the information management system 40 may be configured by a single information processing device 41. In this case, the database DB is stored in memory 142. The information management system 40 may also be equipped with other devices that perform processing between the information processing device 41 and the database device 42. In this case, the three devices of the information management system 40 constitute a so-called web 3-tier structure.

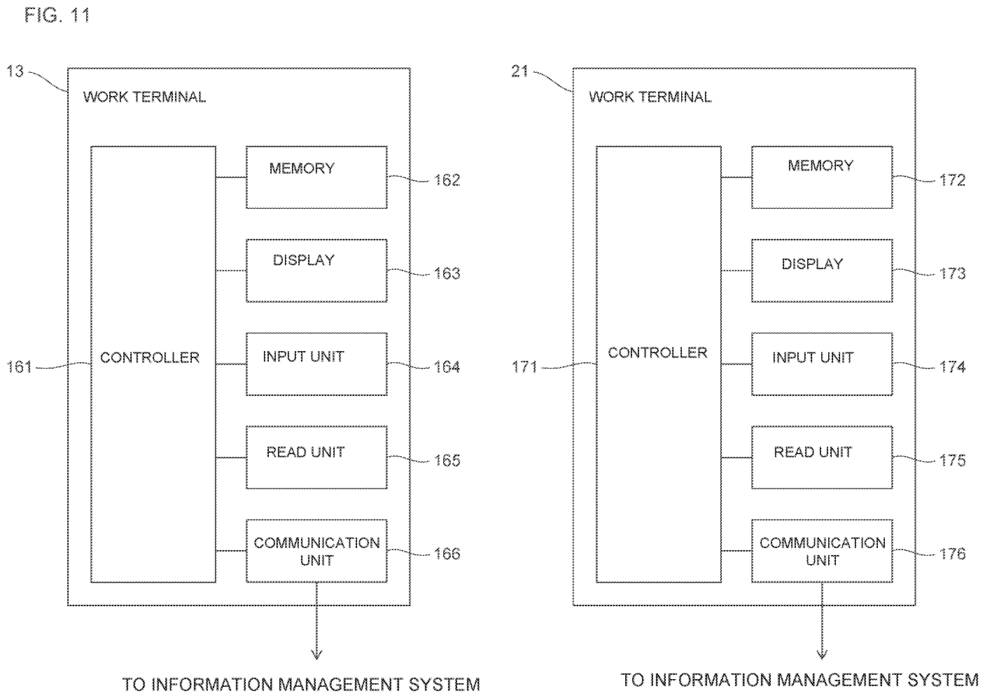

FIG. 11 is a block diagram showing the functional configuration of work terminal 13 located at facility 10 and work terminal 21 located at device center 20.

The work terminals 13 and 21 comprise, for example, a personal computer or a smart phone. The work terminal 13 has a controller 161, a memory 162, a display 163, an input unit 164, a reading unit 165, and a communication section 166. Work terminal 21 is equipped with a controller 171, a memory 172, a display 173, an input unit 174, a reading unit 175, and a communication section 176. Since work terminals 13 and 21 have almost the same configuration and functions as each other, the configuration of work terminal 13 will be mainly described below for convenience.

The controller 161 includes, for example, a CPU. The controller 161 controls each hardware section of the terminal and executes various processes by executing a computer program stored in the memory 162. The memory 162 includes, for example, an SSD or HDD.

Display 163 includes, for example, an LCD display. The input unit 164 includes, for example, a keyboard and a mouse. Instead of the display 163 and the input unit 164, a touch panel display may be used. The input unit 164 may be, for example, a microphone. For example, at the work terminal 21, a voice recognition program may be stored in the memory 172 so that the operator of the device center 20 can input information and instructions without touching the input unit 174. This can prevent contamination of the surgical instruments 30 by bacteria adhesion, etc. caused by the operator touching the input unit 174 in the sterilization process (see FIG. 20) described below. The work terminal 21 may further include a speaker, and information displayed on the display 173 may be output audibly through the speaker. This allows the operator to perform maintenance work without having to check the display 173, thereby making maintenance work more efficient.

If a bar code is printed on the surface of the tag 35 (see FIG. 7), the reading unit 165 comprises a bar code reader, and if a QR code (registered trademark) is printed on the surface of the tag 35, the reading unit 165 comprises a camera. The communication section 166 includes, for example, an interface capable of communicating with the information management system 40 based on the Ethernet standard.

The controller 161 receives information for configuring screens from the information management system 40 by executing a web browser or a predetermined application. The controller 161 displays various screens on the display 163 based on the information for configuring the received screens.

The operator of facility 10 who operates work terminal 13 operates the buttons, etc. included in the screen displayed on display 163 via input unit 164. Similarly, the operator of the device center 20 who operates the work terminal 21 operates the buttons, etc. included in the screen displayed on the display 173 via the input unit 174. As a result, various information is transmitted from the work terminals 13 and 21 to the information management system 40.

FIG. 12 schematically shows the details of the structure and status IDs of the surgical instrument table stored in the database DB.

As shown in the top row of FIG. 12, the surgical instrument table contains the following items: surgical instrument ID, type ID of the surgical instruments 30, surgical instrument name, status ID, and last updated date.

The surgical instrument ID, type ID, and surgical instrument name items correspond to the surgical instrument ID, type ID, and surgical instrument name shown in FIG. 5. That is, the surgical instrument ID item indicates an ID that can individually identify the surgical instruments 30. The type ID item indicates an ID that can individually identify the type of the relevant surgical instrument 30. The item of the surgical instrument name indicates the function of the relevant surgical instrument 30 and the type of end effector 33.

The Status ID item indicates status information regarding the status of the surgical instrument 30. The status ID is set to the status ID value A to E, as shown in the lower part of FIG. 12. The status ID value A (in stock) indicates that the surgical instruments 30 have completed the maintenance process at the device center 20 and are stored in the warehouse. The status ID value B (shipping determined) indicates that the surgical instrument 30 has been determined for shipping at the device center 20. Status ID value C (shipping) indicates that the surgical instrument 30 is being delivered from the device center 20 to the facility 10. Status ID value D (collection) indicates that the surgical instrument 30 is being collected from the facility 10 to the device center 20. The status ID value E (under maintenance) indicates that the surgical instrument 30 is in the maintenance process at the device center 20. The Last Update Date and Time field indicates the date and time when the status ID in the surgical instrument table was last updated.

The surgical instrument table contains information on all of the surgical instruments 30 used in the surgical instrument management system 1. For convenience, the top row of FIG. 12 illustrates information on surgical instruments 30 whose surgical instrument IDs are SI00001 to SI00003. When a new surgical instrument 30 is introduced into the surgical instrument management system 1, the surgical instrument ID, type ID, and name of the surgical instrument 30 are registered in the surgical instrument table. The status ID and the last update date of the surgical instruments 30 are updated according to the use of the surgical instruments 30. When a surgical instrument 30 is discarded, the information corresponding to the surgical instrument 30 is deleted from the surgical instrument table.

The surgical instrument table may further include a registration date entry indicating the date when the surgical instrument 30 was first registered with the device center 20. This makes it possible to grasp the elapsed period from the registration date to the present, the elapsed period from the registration date to the last update date, etc., and to use them as a reference to determine whether the device center 20 should continue to manage or discard the surgical instruments 30 whose number of usage or cumulative usage time have not reached the upper limit, etc.

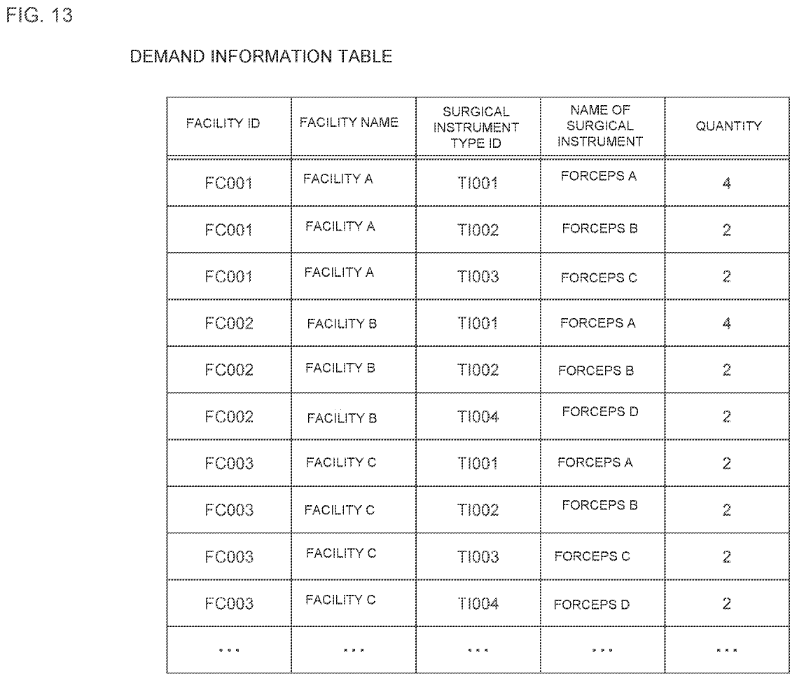

FIG. 13 schematically shows the composition of the demand information table stored in the database DB.

The information management system 40 determines the type and quantity of the surgical instruments 30 required at each facility 10 based on the surgery schedule information (see FIG. 3) transmitted from the work terminal 13 at each facility 10. The information management system 40 adds a record to the demand information table based on the received surgery schedule information and the determined type and quantity of the surgical instruments 30.

As shown in FIG. 13, the demand information table contains items for facility ID, facility name, type ID of the surgical instruments 30, surgical instrument name, and number. The facility ID item indicates an ID that can identify each of the facilities 10 individually. The facility name item indicates the name of the facility 10. The number item indicates the required quantity of surgical instruments 30 of the type corresponding to the type ID at the facility 10 corresponding to the facility ID.

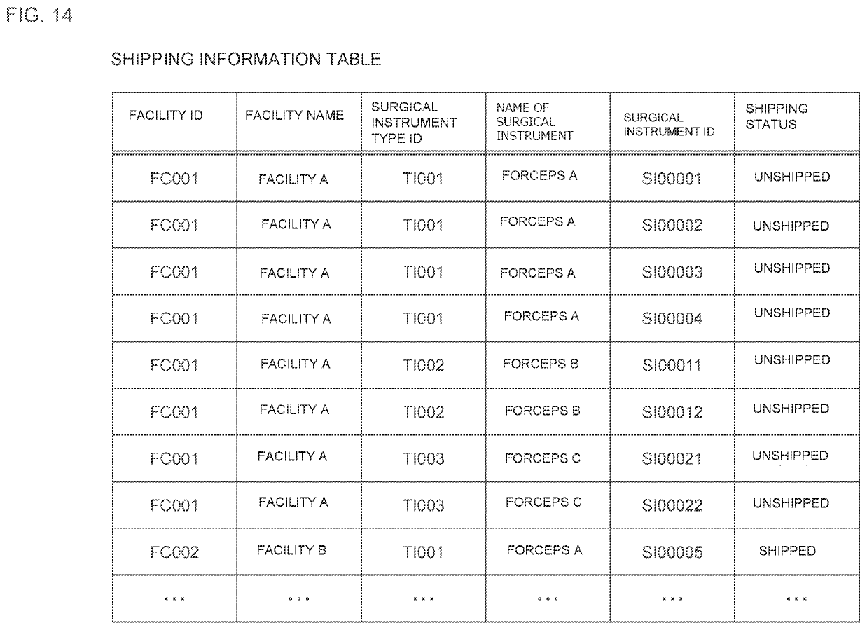

FIG. 14 schematically shows the structure of the shipping information table stored in the database DB.

The information management system 40 adds a record to the shipping information table based on the surgical instruments 30 set as determined for shipping and the facility 10 to which the surgical instruments 30 are to be delivered.

As shown in FIG. 14, the shipping information table contains items for facility ID, facility name, type ID of the surgical instruments 30, surgical instrument name, surgical instrument ID, and shipping status. The shipping status item indicates whether the shipping procedure for the surgical instruments 30 has not yet been completed (unshipped) or whether the shipping procedure for the surgical instruments 30 has already been completed (shipped).

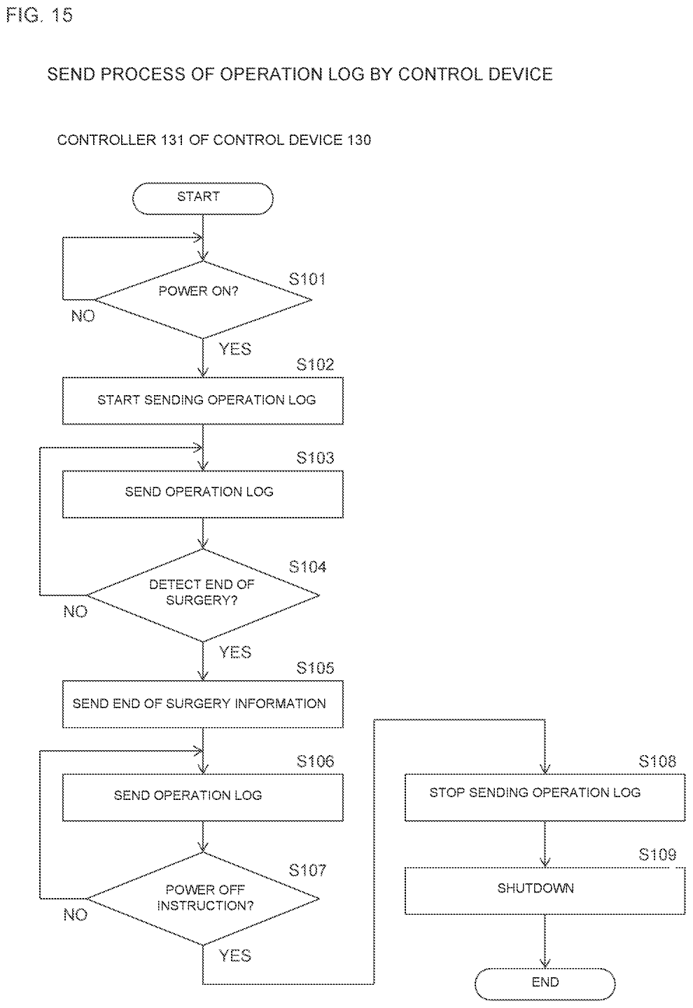

The flowcharts and screens shown in FIGS. 15 to 27 will now describe the processes performed by the surgical instrument management system 1.

FIG. 15 is a flowchart showing the process of transmitting the operation log by the control device 130.

When the surgical robot system 11 is turned on when surgery is started at facility 10 (step S101: YES), in step S102, the controller 131 of the control device 130 starts transmitting the operation logs corresponding to the four arms 115. The controller 131 transmits the operation logs to the information management system 40 via the communication control device 12 at predetermined time intervals (e.g., 1 second) in step S103 until the end of the surgery is detected.

The controller 131 detects the end of the surgery by detecting a predetermined state change, such as removal of the drape 115a from the surgical robot system 11. When the controller 131 detects the end of the surgery (step S104: YES), it transmits the surgery end information indicating that the surgery has ended to the information management system 40 via the communication control device 12 in step S105, and continues transmitting the operation log in step S106. When an instruction to turn off the power is input from the surgical robot system 11 (step S107: YES), the controller 131 terminates, in step S108, the transmission of the operation log started in step S102, and in step S109, the surgical robot system 11 is shut down the system.

The end of the surgery may be detected by the controller 141 or 151 of the information management system 40 based on the operation log received from the control device 130. In that case, when the controller 141 or 151 of the information management system 40 detects the end of the surgery, it stores the date and time of the end of the surgery in the memory 142 or 152 associated with the surgical robot ID of the corresponding surgical robot system 11.

FIG. 16 schematically illustrates the configuration of the operation log for arm 115 to which the surgical instrument 30 is attached.

The operation log contains items for date and time, surgical robot system ID, arm position, surgical instrument ID, and operation flags. The date and time item indicates the date and time when the operation log was transmitted. The item of surgical robot system ID indicates an ID that can identify the surgical robot system 11 individually. The arm position item indicates which of the three arms 115 the information contained in the operation log is based on. The surgical instrument ID item is the surgical instrument ID of the surgical instruments 30 attached to the arm 115 indicated by the arm position. The operation flag is a flag indicating that the surgical instrument 30 attached to the arm 115 was in an operational state.

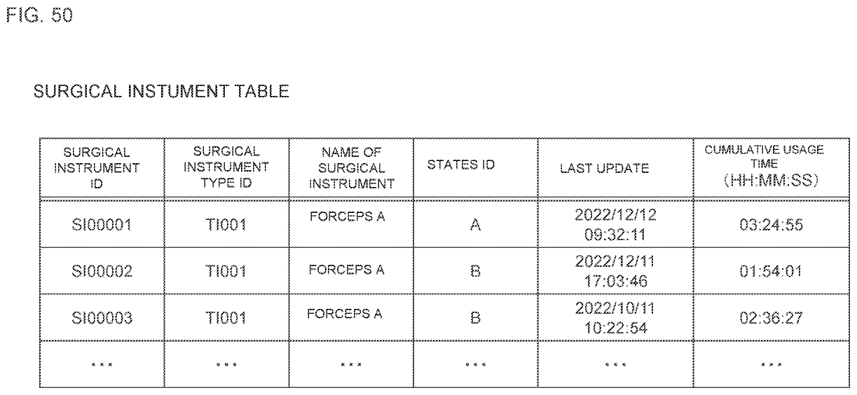

Specifically, the surgical robot system 11 and the information management system 40 may be configured that, when an operator, for example a physician, operates the hand controller 126 or foot unit 127 of the operation apparatus 120 to operate the surgical instrument 30, an operation log indicating the operation is sent from the control device 130 to the information management system 40, and the operation flag corresponding to the date and time and to the surgical instrument 30 is set to “1”. This allows the controller 141 of the information management system 40 to obtain the cumulative usage time (see FIG. 50) described below by calculating the number of times the operation flag is set to “1”.

The value of the operation flag may be set according to the type of operation of the surgical instrument 30 (e.g., opening and closing forceps, energizing an electric scalpel), and the cumulative usage time may be acquired for each type of operation. The method of obtaining the cumulative usage time is not limited to this method and may be, for example, the total time the surgical instrument 30 is attached to the arm 115, regardless of whether the surgical instrument 30 is operating or not.

The operation log transmitted at predetermined time intervals corresponds to one line of data in FIG. 16. The example shown in FIG. 16 shows an operation log for arm 1 in the surgical robot system 11 whose surgical robot system ID is RB001. When the controller 141 of the information management system 40 receives the operation log continuously for each predetermined period, it stores the received operation log in the database DB. In FIG. 16, the operation logs are arranged in chronological order from top to bottom for convenience.

When the surgical robot system 11 is turned on, for example, the operation log shown in lines 1 and 2 of FIG. 16 is transmitted. At this time, if the surgical instrument 30 is not attached to the first arm, the surgical instrument ID entry is assigned information that the surgical instrument 30 is not attached (e.g., “-” or null). Subsequently, when the surgical instrument 30 is attached to the first arm, an operation log including the surgical instrument ID is sent, as shown in lines 4 and 5 of FIG. 16. When the surgical instrument 30 is subsequently removed from the first arm, the operation log no longer includes the surgical instrument ID, as shown in lines 7 and 8 of FIG. 16. If another surgical instrument 30 is subsequently attached to arm 1, the operation log including the surgical instrument ID of the other surgical instrument 30 is transmitted, as shown in lines 10 and 11 of FIG. 16. As shown in lines 1 to 3 from the bottom of FIG. 16, the operation log continues to be transmitted after the surgery is completed. After that, when the instruction to turn off the surgical robot system 11 is input, the transmission of the operation log is terminated.

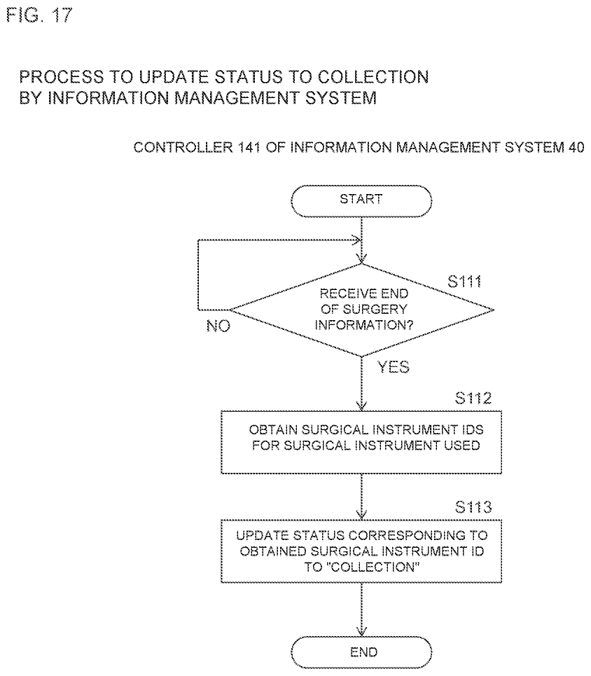

FIG. 17 is a flowchart showing the process of updating status to collection by the information management system 40.

When the controller 141 of the information management system 40 receives the surgical operation end information transmitted from the control device 130 in step S105 of FIG. 15 (step S111: YES), it obtains the surgical instrument ID of the surgical instrument 30 used in that operation in step S112. Here, the surgical operation end information includes the surgical robot system ID and the date and time when the surgery was completed. The controller 141 reads from the database DB the operation log for the period corresponding to the surgical robot system ID included in the surgery completion information and corresponding to the surgery completion date and time included in the surgery completion information, and obtains the surgical instrument IDs of the surgical instruments 30 used in the relevant surgery based on the read operation log.

In step S113, the controller 141 updates the status corresponding to the surgical instrument ID obtained in step S112 to “collection”. Specifically, the controller 141 updates the value of the status ID corresponding to the surgical instrument ID obtained in step S112 to D in the surgical instrument table shown in the upper row of FIG. 12.

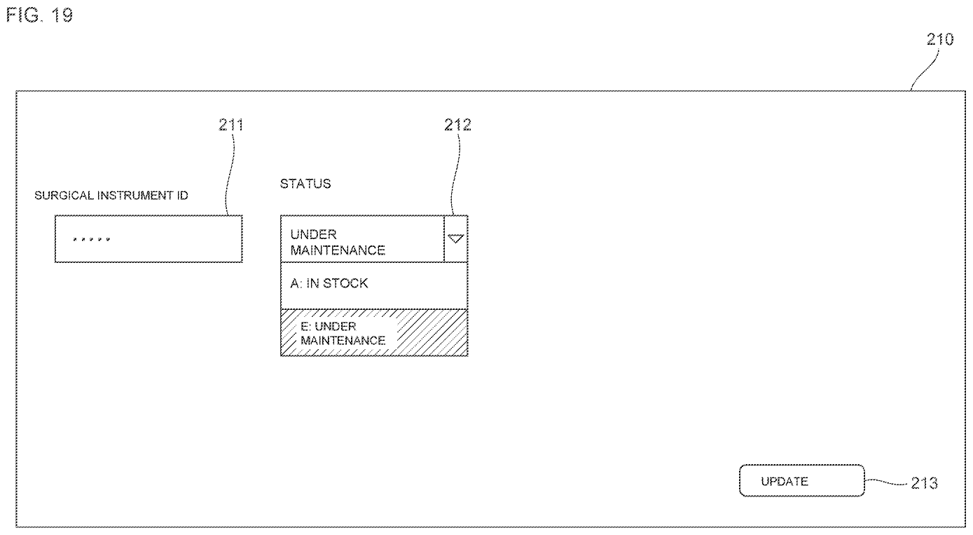

The surgical instrument table shown in the top row of FIG. 12 may further include an entry for a use flag indicating that the surgical instruments 30 were used in the surgery. The initial value of the use flag is, for example, “0”. In that case, the controller 141 sets the use flag corresponding to the surgical instrument ID to “1” for the surgical instrument 30 for which the surgical instrument ID was obtained in step S112. If the status is set to “A: In stock” on the update screen 210 (see FIG. 19) described below, and that information is sent to the information management system 40, the controller 141 sets the use flag to “0”. This allows the operator of the device center 20, when performing maintenance of the surgical instruments 30 in the maintenance process, to know whether the collected surgical instruments 30 have been used for surgery or not, thereby making the maintenance work more efficient.

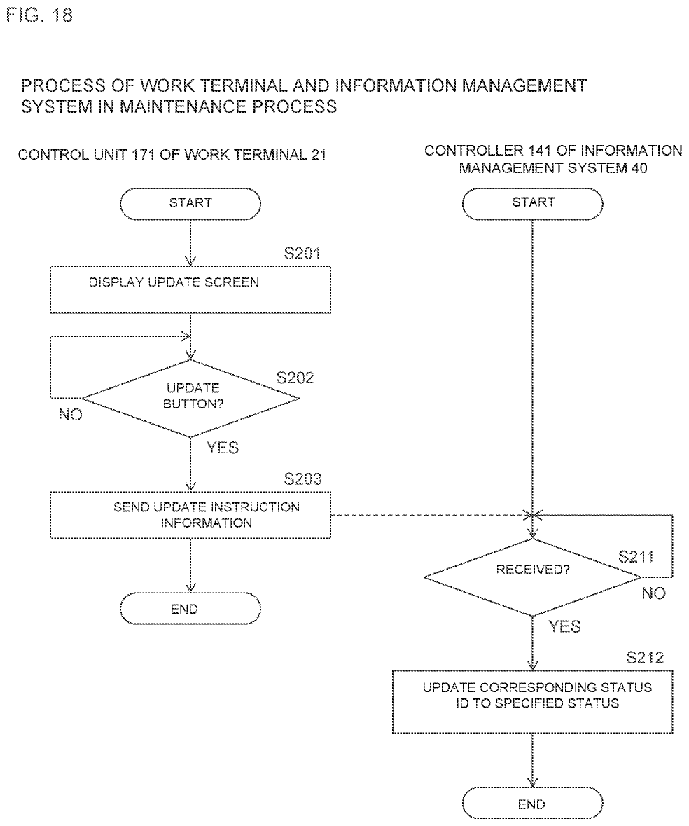

FIG. 18 is a flowchart showing the processing of work terminal 21 and information management system 40 in the maintenance process of device center 20.

The operator in charge of the maintenance process of the device center 20 operates the work terminal 21 to update the status of the surgical instruments 30 to be processed. Specifically, the operator operates the input unit 174 of the work terminal 21 to input instructions to display the update screen 210 (see FIG. 19) for updating the status. When the controller 171 of the work terminal 21 receives the display instruction, the update screen 210 is displayed on the display 173 in step S201.

FIG. 19 schematically illustrates the update screen 210.

The update screen 210 has a text box 211, a pull-down menu 212, and an update button 213.

The operator uses the reading unit 175 of the work terminal 21 to read the tag 35 of the surgical instrument 30 and enter the surgical instrument ID into the text box 211. Alternatively, the operator refers to the string of the surgical instrument ID on the surface of the tag 35 and enters the surgical instrument ID into the text box 211 using the input unit 174 of the work terminal 21. The operator also selects the updated status of the surgical instrument 30 using the pull-down menu 212. The pull-down menu 212 is configured to allow selection of in stock or under maintenance as the status. When the update button 213 is operated by the operator, the status ID corresponding to the surgical instrument ID entered in the text box 211 is updated to the status ID selected in the pull-down menu 212.

The operator typically displays the update screen 210 on the display 173 when the operator receives the surgical instruments 30 collected from the facility 10 and when the maintenance process for the surgical instruments 30 is completed. The operator selects “E: Under Maintenance” from the pull-down menu 212 when receiving the surgical instruments 30 collected from the facility 10, and selects “A: In stock” from the pull-down menu 212 when the maintenance process for the surgical instruments 30 is complete.

If the work terminal 21 is configured to allow voice input, for example, the status ID may be updated to “A: In stock” by the operator voice instructing the input unit 174, which includes a microphone, to update status “A: In stock”.

Returning to FIG. 18, when the update button 213 is operated by the operator (step S202: YES), in step S203, the controller 171 of the work terminal 21 sends update instruction information including the status ID of the surgical instrument ID entered in the text box 211 and the status ID selected in the pull-down menu 212 to the information management system 40.

When the controller 141 of the information management system 40 receives the update instruction information from the work terminal 21 (step S211: YES), in step S212, it updates the status ID of the surgical instrument ID in the surgical instrument table (see FIG. 12) corresponding to the surgical instrument ID included in the update instruction information to the status ID included in the update instruction information. This allows the status of the surgical instruments 30 collected from the facility 10 to be set to under maintenance and the status of the surgical instruments 30 whose maintenance has been completed to be set to in stock.

FIG. 20 is a flowchart showing the operator's work in the maintenance process of the device center 20.

The maintenance process includes a cleaning process consisting of steps S301 to S305 and a sterilization process consisting of steps S306 and S307. Each process is performed by an operator in charge of the maintenance process.

In step S301, the operator connects the hose 39 to the cleaning liquid supply ports 36a and 36b and supplies cleaning water with water pressure to the cleaning liquid supply ports 36a and 36b. This washes the inside of the surgical instrument 30, as described with reference to FIG. 7.

In step S302, the operator washes the entire tip of the surgical instrument 30 (shaft 32 and end effector 33) with the surgical instrument 30 immersed in the wash water. In step S303, the operator brushes the surgical instruments 30 with a soft bristle nylon brush while the surgical instruments 30 are immersed in the wash water or the wash water is poured over the surgical instruments 30. In step S304, the operator inspects the surgical instruments 30 for residue using a magnifying glass, and repeats the process of steps S301 to S303 if there is any residue. In step S305, the operator automatically cleans the surgical instruments 30 using a washer-disinfector or other cleaning device.

As shown in steps S301 to S305, the reason why the cleaning process consists of multiple steps is because the surgical instruments 30 have a complex structure and are prone to residual blood, body fluids, and tissue adhered by the surgery. According to the cleaning process comprising steps S301 to S305, the surgical instruments 30 can be cleaned reliably.

In step S306, the operator packs the cleaned surgical instruments 30 in a sealed condition in a tray or other container under conditions where sterility is assured. In step S307, the operator performs heat sterilization treatment using high-pressure steam. The heat sterilization process is performed under sterilization conditions such as sterilization temperature of 134° C., exposure time of 3 minutes, and drying time of 30 to 60 minutes. In step S307, gas sterilization with ethylene oxide gas or plasma sterilization, for example, may be performed instead of high-pressure steam sterilization.

As described above, the maintenance process consists of multiple processes, and if maintenance of the surgical instruments 30 is performed at the facility 10, the burden of maintenance of the surgical instruments 30 becomes large for the facility 10. In contrast, in embodiment 1, since maintenance is performed at the device center 20, the burden of maintenance of the surgical instruments 30 can be reduced at the facility 10 using the surgical robot system 11.

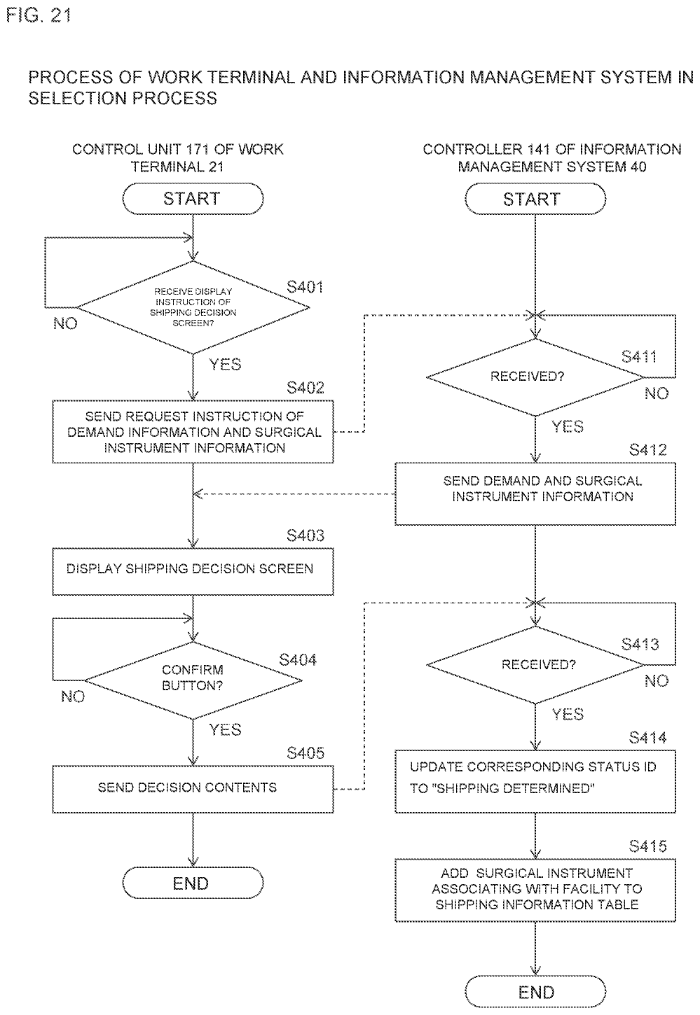

FIG. 21 is a flowchart showing the process of work terminal 21 and information management system 40 in the selection process of device center 20.

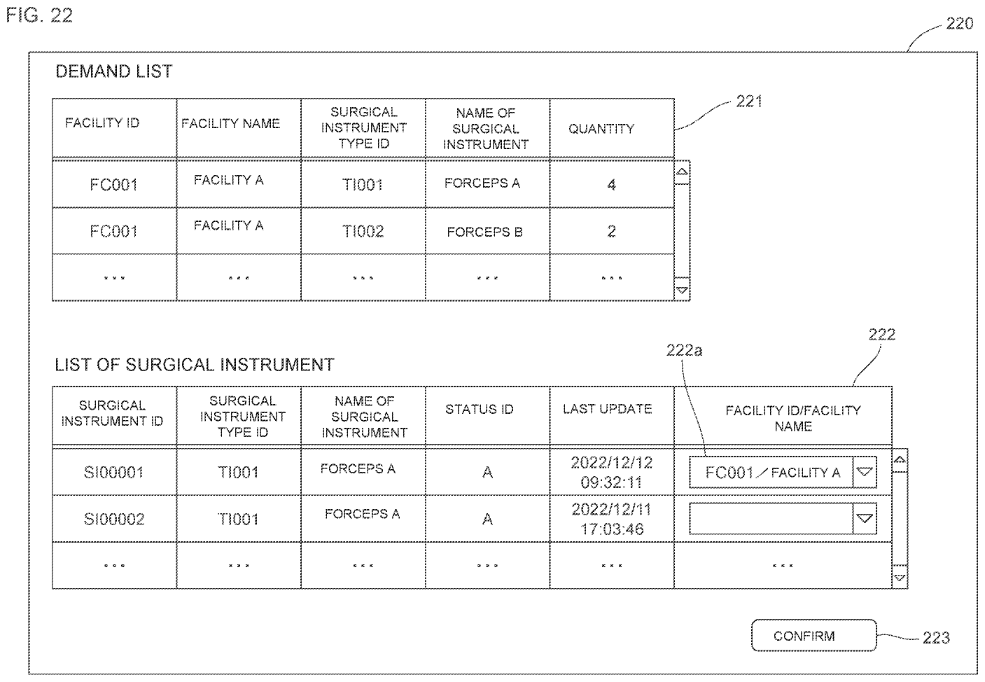

The operator in charge of the selection process at the device center 20 inputs an instruction to display the shipping decision screen 220 (see FIG. 22). When the controller 171 of the work terminal 21 accepts the instruction to display the shipping decision screen 220 (step S401: YES), it transmits in step S402 to the information management system 40 a request instruction for demand information and surgical instrument information for the surgical instrument 30 that can be selected.

When the controller 141 of the information management system 40 receives the request instruction sent in step S402 (step S411: YES), it sends the demand information and surgical instrument information to the work terminal 21 that sent the request instruction in step S412.

Specifically, the controller 141 generates demand information indicating the type and quantity of surgical instruments 30 that need to be delivered to each facility 10 based on the demand information table (see FIG. 13). The demand information includes the facility ID, the name of the facility, the type ID of the surgical instruments 30, the name of the surgical instruments, and the quantity of surgical instruments 30. The controller 141 also generates surgical instrument information indicating the surgical instruments 30 that can be selected for shipping based on the surgical instrument table (see FIG. 12), i.e., the surgical instruments 30 whose status ID value is A (in stock). The surgical instrument information includes the surgical instrument ID, the type ID of the surgical instruments 30, the name of the surgical instrument, the status ID, and the last update date and time. If the surgical instrument table (see the upper part of FIG. 12) further includes a registration date item, the surgical instrument information may further include the registration date. The controller 141 then transmits the generated demand information and surgical instrument information to the work terminal 21.

In step S403, the controller 171 of the work terminal 21 receives the demand information and surgical instrument information from the information management system 40, and displays the shipping decision screen 220 on the display 173 of the work terminal 21.

FIG. 22 schematically illustrates the shipping decision screen 220 displayed on the display 173 of the work terminal 21 in the device center 20.

The shipping decision screen 220 has a demand list 221, a surgical instrument list 222, and a confirm button 223.

The demand list 221 is based on the demand information transmitted from the information management system 40. The demand list 221 contains the same items as the demand information table shown in FIG. 13.

The surgical instrument list 222 is displayed based on the surgical instrument information transmitted from the information management system 40. The surgical instruments 30 displayed in the surgical instrument list 222 are the surgical instruments 30 that can be selected for shipping, i.e., those with a status ID value of A (in stock). In addition to the same items as in the surgical instrument table shown in FIG. 12, the surgical instrument list 222 includes items indicating the facility ID and the name of the facility. If the surgical instrument table (see the upper part of FIG. 12) further includes an entry for the registration date, the surgical instrument list 222 may further include the registration date. A pull-down menu 222a is placed for each surgical instrument 30 in the item indicating the facility ID and facility name. The pull-down menu 222a is configured to allow selection of any one of all facilities 10 or a state in which no facility 10 is selected (blank).

The operator of device center 20 confirms the type and quantity of surgical instruments 30 required at each facility 10 by referring to the demand list 221, and operates the pull-down menu 222a of the surgical instruments list 222 to map the surgical instruments 30 to be delivered to the destination facility 10. When the operator has completed the operation of the pull-down menu 222a, he or she operates the confirm button 223.

Returning to FIG. 21, when the controller 171 of the work terminal 21 accepts the operation of the confirm button 223 on the shipping decision screen 220 (step S404: YES), in step S405, the decision contents including the mapping between the surgical instruments 30 and the facility 10 set in the surgical instruments list 222 of the shipping decision screen 220 are transmitted to the information management system 40.

When the controller 141 of the information management system 40 receives the decision contents from the work terminal 21 (step S413: YES), in step S414, in the surgical instrument table, the status ID value in the surgical instrument table (see FIG. 12) corresponding to the surgical instrument ID included in the decision contents is updated to B (shipping determined). In step S415, the controller 141 adds a record to the shipping information table (see FIG. 14) that associates the surgical instrument 30 in question with the shipping destination facility 10 based on the decision contents received. At this time, the shipping status item of the added record is set to undelivered.

The demand information table (see FIG. 13) may be updated after the process shown in FIG. 21 has been used to map the surgical instruments 30 to the shipping facility 10. In this case, a notification may be sent from the information management system 40 to the work terminal 21 via a web browser or prescribed application, indicating that a new demand for the surgical instruments 30 has occurred.

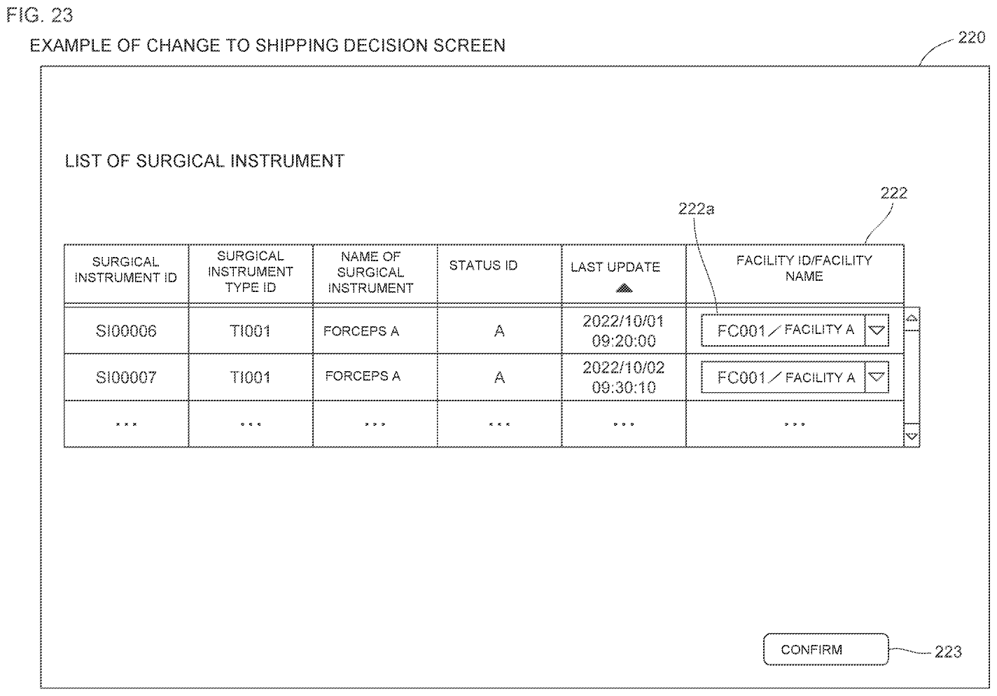

Although the demand list 221 corresponding to the demand information was displayed on the shipping decision screen 220 in FIG. 22, the demand list 221 may be omitted. In this case, for example, a partially modified surgical instrument list 222 from FIG. 22 is displayed, as shown in the modified example in FIG. 23.

FIG. 23 schematically illustrates the modified shipping decision screen 220.

In this modified example, the demand list 221 is omitted and the pull-down menu 222a is configured to allow only the facility 10 corresponding to the type of surgical instrument 30 in question to be selected based on the demand information. For example, if only forceps A and forceps B are needed at facility A, the pull-down menu 222a corresponding to forceps A and forceps B is configured so that facility A can be selected, but the pull-down menu 222a corresponding to other surgical instruments 30 such as forceps C is configured so that the facility A cannot be selected. This allows the operator to map the surgical instruments 30 to facility 10 without having to refer to the demand information.

In FIG. 23, each row of the surgical instrument list 222 is automatically sorted in the order of the date and time of the last update. In other words, in the surgical instrument list 222, the surgical instruments 30 are sorted in the order of the date and time that the status was set to in stock. Generally, it is not desirable for surgical instruments 30 to be stored in a warehouse for a long period of time. According to this configuration, the operator can easily determine the surgical instruments 30 that have been stored for a long period of time to be delivered by mapping the surgical instruments 30 and the facility 10 in order from the top of the surgical instruments list 222, thereby preventing the surgical instruments 30 from being stored in the warehouse for a long period of time.

In FIG. 23, the facility 10 is pre-selected in the pull-down menu 222a based on the demand and surgical instrument information. This allows the operator to map the surgical instruments 30 to the facilities 10 simply by operating the confirm button 223, without the need to select a facility 10 for each surgical instrument 30.

The configuration and automatic selection of the pull-down menu 222a and the sorting of the surgical instrument list 222, as shown in FIG. 23, may be performed by the controller 171 of the work terminal 21 based on the received demand information and selectable instruments information, or the controller 141 of the information management system 40 may generate information for displaying the shipping decision screen 220 beforehand and the controller 171 of the work terminal 21 may display the shipping decision screen 220 based on the information.

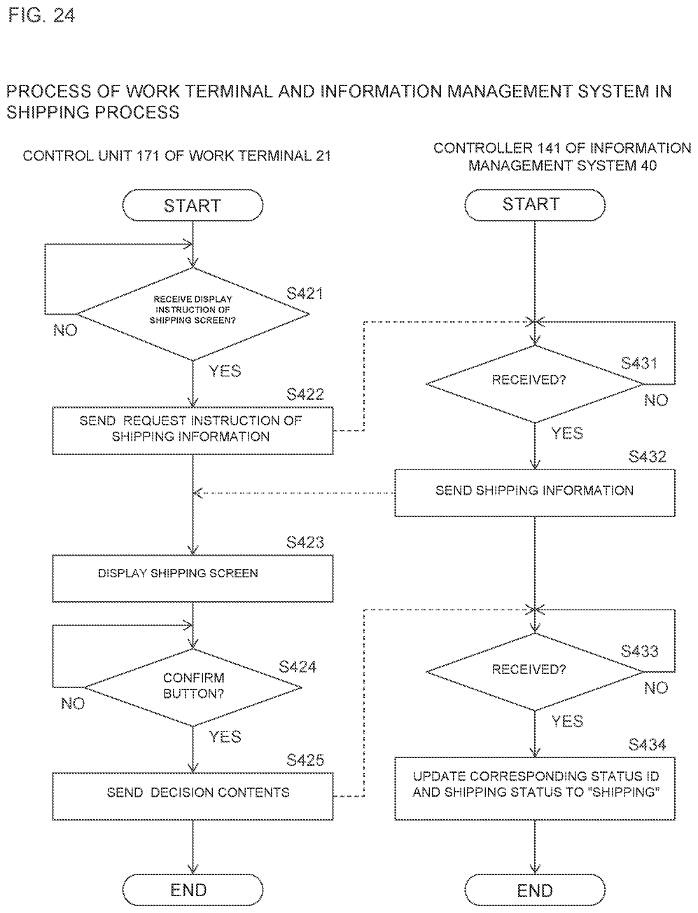

FIG. 24 is a flowchart showing the processing of work terminal 21 and information management system 40 in the shipping process of device center 20.

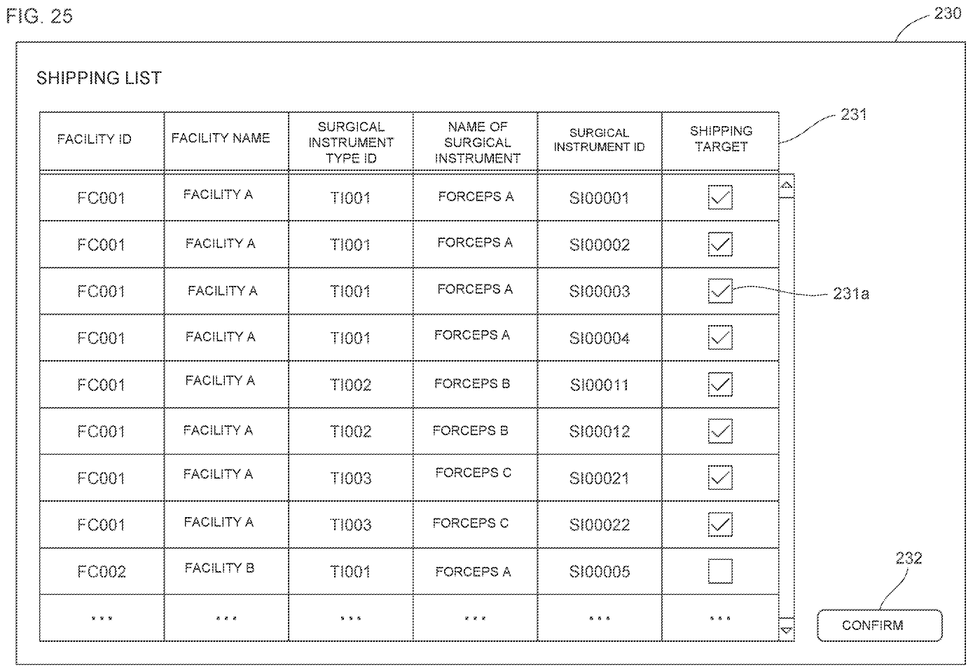

The operator in charge of the shipping process at the device center 20 inputs an instruction to display the shipping screen 230 (see FIG. 25). When the controller 171 of the work terminal 21 accepts the instruction to display the shipping screen 230 (step S421: YES), it sends an instruction to request shipping information to the information management system 40 in step S422.

When the controller 141 of the information management system 40 receives the request instruction sent in step S422 (step S431: YES), it sends the shipping information to the work terminal 21 that sent the request instruction in step S432. Specifically, the controller 141 generates shipping information including the shipping status of the surgical instruments 30 determined to be delivered to each facility 10 based on the shipping information table (see FIG. 14). The controller 141 then transmits the generated shipping information to the work terminal 21.

In step S423, the controller 171 of the work terminal 21 receives the shipping information from the information management system 40 and displays the shipping screen 230 on the display 173 of the work terminal 21.

FIG. 25 schematically illustrates the shipping screen 230 displayed on the display 173 of the work terminal 21 in the device center 20.

The shipping screen 230 has a shipping list 231 and a confirm button 232.

The shipping list 231 is displayed based on the shipping information transmitted from the information management system 40. The surgical instruments 30 displayed in the shipping list 231 are the surgical instruments 30 whose status ID values are B (shipping determined) or C (shipping). The shipping list 231 contains the same items as the shipping information table shown in FIG. 14. A check box 231A is placed for each surgical instrument 30 in the item for shipping. If the status of the surgical instrument 30 is shipping determined, check box 231a is unchecked; if the status of the surgical instrument 30 is shipping, check box 231a is checked.

The operator of the device center 20 changes the check box 231a of the surgical instruments 30 for which the check box 231a is “unchecked” to “checked” for the shipping procedure, and operates the confirm button 232.

Only surgical instruments 30 whose status is shipping determined may be displayed in the shipping list 231. However, if the surgical instruments 30 whose status is shipping are displayed as shown in FIG. 25, the operator can see which surgical instruments 30 have already been delivered in a given shipping period.

Returning to FIG. 24, when the controller 171 of the work terminal 21 accepts the operation of the confirm button 232 on the shipping screen 230 (step S424: YES), in step S425, the decision contents including the surgical instrument ID that has been newly checked in the shipping list 231 on the shipping screen 230 are transmitted to the information management system 40.

When the controller 141 of the information management system 40 receives the decision contents from the work terminal 21 (step S433: YES), it updates the status ID value in the surgical instrument table (see FIG. 12) corresponding to the surgical instrument ID included in the decision contents to C (shipping) in step S434. In addition, the controller 141 updates the shipping status corresponding to the surgical instrument ID included in the decision contents to shipping in the shipping information table.

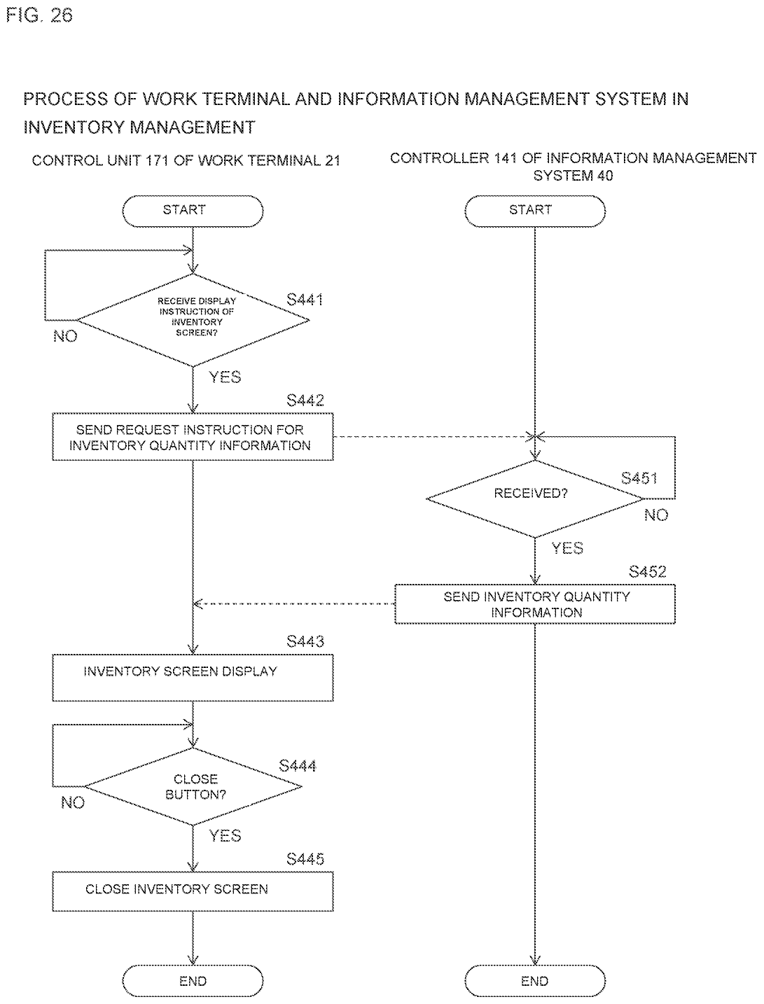

FIG. 26 is a flowchart showing the process of work terminal 21 and information management system 40 during inventory management at device center 20.

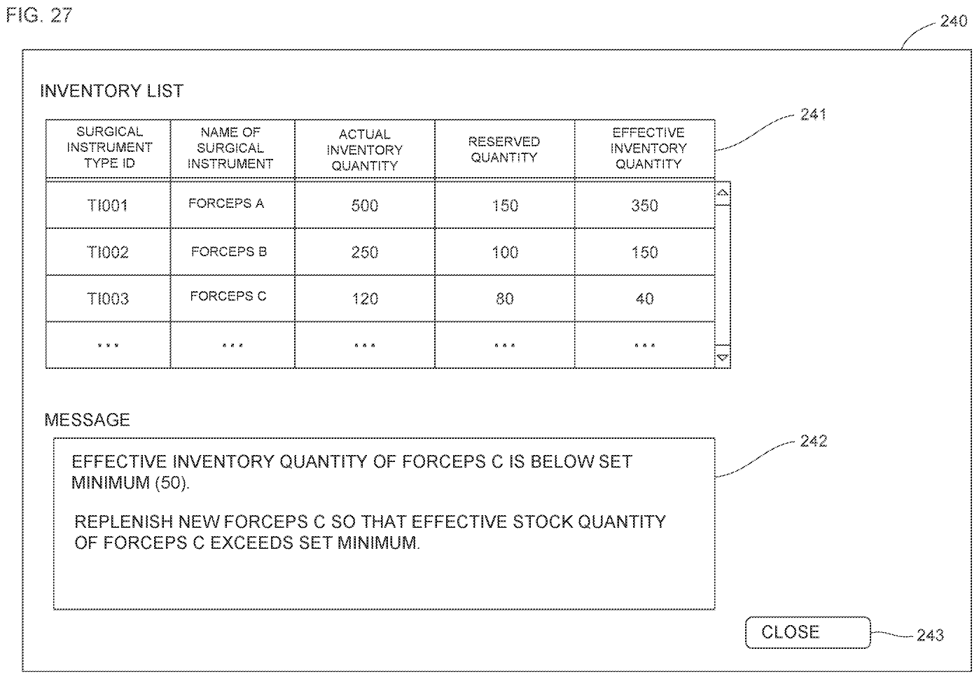

The operator in charge of the shipping process at the device center 20 inputs an instruction to display the inventory screen 240 (see FIG. 27). When the controller 171 of the work terminal 21 accepts the instruction to display the inventory screen 240 (step S441: YES), it sends an instruction to request inventory count information to the information management system 40 in step S442.

When the controller 141 of the information management system 40 receives the request instruction transmitted in step S442 (step S451: YES), it transmits the inventory count information to the work terminal 21 that transmitted the request instruction in step S452. Specifically, the controller 141 calculates the quantity of surgical instruments 30 whose status ID value is A (in stock) and the quantity of surgical instruments 30 whose status ID value is B (shipping determined) for each type ID of surgical instruments 30 based on the surgical instruments table (see FIG. 12). The controller 141 then transmits the above numbers for each type ID of the surgical instruments 30 generated to the work terminal 21 as the inventory count information.

In step S443, the controller 171 of the work terminal 21 receives the inventory count information from the information management system 40 and displays the inventory screen 240 on the display 173 of the work terminal 21.

FIG. 27 schematically illustrates the inventory screen 240 displayed on the display 173 of the work terminal 21 in the device center 20.

The inventory screen 240 has an inventory count list 241, a message area 242, and a close button 243.

The inventory count list 241 is displayed based on the inventory count information transmitted from the information management system 40. The inventory count list 241 includes items for the type ID of the surgical instrument 30, the name of the surgical instrument, the actual inventory quantity, the reserved quantity, and the effective inventory count. The item of actual inventory quantity indicates the sum of the quantity of surgical instruments 30 whose status ID value is A (in stock) and the quantity of surgical instruments 30 whose status ID value is B (shipping determined). The item “Reserved quantity” indicates the quantity of surgical instruments 30 whose status ID value is B (shipping determined). The item of the effective inventory count indicates the actual inventory quantity minus the reserved quantity. The inventory count list 241 displays the actual inventory quantity, the reserved quantity, and the effective inventory count for each type of surgical instrument 30.

In message area 242, a warning message regarding the inventory count is displayed. In the example of FIG. 27, a message indicating that the effective inventory quantity of forceps C is below a preset lower limit (e.g., 50) and a message urging replenishment of new forceps C are displayed. The message displayed in the message area 242 may be output audibly via a speaker instead of or in addition to the display.

By referring to the inventory count list 241 and message area 242, the operator of device center 20 can see, for example, that the effective inventory count is decreasing due to a rapid increase in the quantity of surgical instruments 30 discarded or reserved, and that new surgical instruments 30 need to be replenished.

Returning to FIG. 26, when the close button 243 is operated (step S444: YES), the controller 171 of the work terminal 21 closes the inventory screen 240 in step S444.

### <Effectiveness of the Method for Managing Surgical Instruments and Information Management System According to Embodiment 1>

As shown in FIGS. 1 to 4, the surgical instrument management system 1 manages surgical instruments 30 that are used in surgical robot systems 11 installed in each of a plurality of facilities 10, collected, maintained including at least cleaning and sterilization, and delivered to one of the facilities 10. As shown in the surgical instruments table in FIG. 12, a status ID (status information regarding the status of the surgical instruments 30) is stored for each of the multiple surgical instruments 30, associated with the surgical instrument ID (identification information) for each of the multiple surgical instruments 30. As shown in FIG. 4, the status ID (status information) corresponding to a surgical instrument 30 that has become available for shipping through maintenance is updated to a status ID (status information, specifically A (in stock)) indicating that it is available for selection for shipping to the facility 10.

According to this configuration, maintenance such as cleaning and sterilization of surgical instruments 30 used in the surgical robot system 11 does not need to be performed at the facility 10 where the surgical robot system 11 is installed, thus reducing the burden on the facility 10 using the surgical robot system 11. In addition, since it is possible to manage whether or not the surgical instruments 30 can be selected for shipping to facility 10 based on the status ID of the surgical instruments 30, surgical instruments 30 that become available for shipping through maintenance can be identified from among multiple surgical instruments 30 and provided to facility 10 for reuse.

The status ID (status information) corresponding to the surgical instruments 30 selected for shipping to facility 10 is updated to a status ID (status information, specifically B (shipping determined)) indicating that they cannot be selected for shipping to facility 10 (step S414 in FIG. 21).

This configuration prevents the surgical instruments 30 corresponding to the same surgical instrument ID from being selected as shipping targets in duplicate.

The surgical instruments 30 are shared by the surgical robotic systems 11 installed in each of the multiple facilities 10.

According to this configuration, the surgical instruments 30 corresponding to the same surgical instrument ID do not become the property of a particular facility 10 and are not used repeatedly only at that facility 10, but are shared among different facilities 10. Therefore, the facility 10 where the surgical robot system 11 is installed does not need to purchase surgical instruments 30 or manage inventory of surgical instruments 30. In addition, the device center 20, etc., which is responsible for storage of surgical instruments 30, does not need to store surgical instruments 30 for each facility 10 where the surgical robot system 11 is installed, thereby reducing the increase in the quantity of surgical instruments 30 stored.

The demand information table shown in FIG. 13 stores demand information regarding the type and quantity of surgical instruments 30 that need to be delivered to the facility 10, and the demand information is provided to the device center 20 (step S412 in FIG. 21) for selecting the surgical instruments 30 to be delivered to the facility 10.

According to this configuration, the device center 20, etc., which is responsible for storing the surgical instruments 30, can smoothly determine the type and quantity of surgical instruments 30 that need to be delivered to the facility 10.

Based on the status ID (status information), surgical instrument information indicating the surgical instruments 30 that can be selected for shipping to the facility 10 is generated (step S412 in FIG. 21), and the surgical instrument information, along with demand information, is provided to the device center 20.

According to this configuration, the device center 20, etc., which is responsible for storing the surgical instruments 30, can smoothly identify the surgical instruments 30 that can be selected for shipping, along with the type and quantity of the surgical instruments 30 that need to be delivered to the facility 10.

Based on the status ID (status information), the type and the quantity of the surgical instruments 30 that can be selected for shipping to the facility 10 are output (step S443 in FIG. 26).

According to this configuration, the inventory of surgical instruments 30 at facility 10 can be managed at device center 20, which is responsible for storage of surgical instruments 30.

As shown in FIGS. 1 and 2, the information management system 40 manages the surgical instruments 30 that are used in the surgical robot systems 11 installed in each of the multiple facilities 10, collected, maintained, including at least cleaning and sterilization, and delivered to one of the facilities 10. The information management system 40 includes a memory 152 (see FIG. 10) that stores a status ID (status information regarding the status of the surgical instruments 30) for each of the plurality of surgical instruments 30 in association with a surgical instrument ID (identification information) for each of the plurality of surgical instruments 30, and the controller 141 (see FIG. 10) that updates the status ID (status information) indicating that the surgical instrument 30 is available to be selected for shipping to the facility 10.

According to this configuration, maintenance such as cleaning and sterilization of surgical instruments 30 used in the surgical robot system 11 does not need to be performed at the facility 10 where the surgical robot system 11 is installed, thus reducing the burden on the facility 10 using the surgical robot system 11. In addition, since it is possible to manage whether or not the surgical instruments 30 can be selected for shipping to facility 10 based on the status ID of the surgical instruments 30, surgical instruments 30 that become available for shipping through maintenance can be identified from among multiple surgical instruments 30 and provided to facility 10 for reuse.

### Modification 1 of Embodiment 1

In embodiment 1, demand information and surgical instrument information were transmitted in step S412 of FIG. 21, but only demand information may be transmitted.

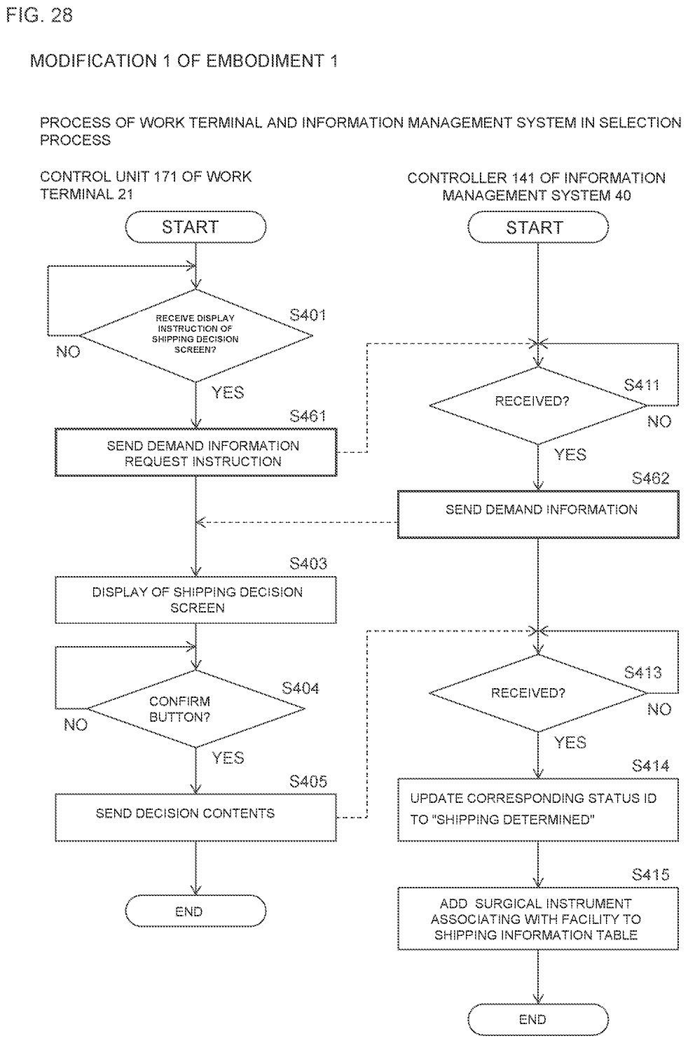

FIG. 28 is a flowchart showing the processing of work terminal 21 and information management system 40 in the selection process of device center 20 for this modification.

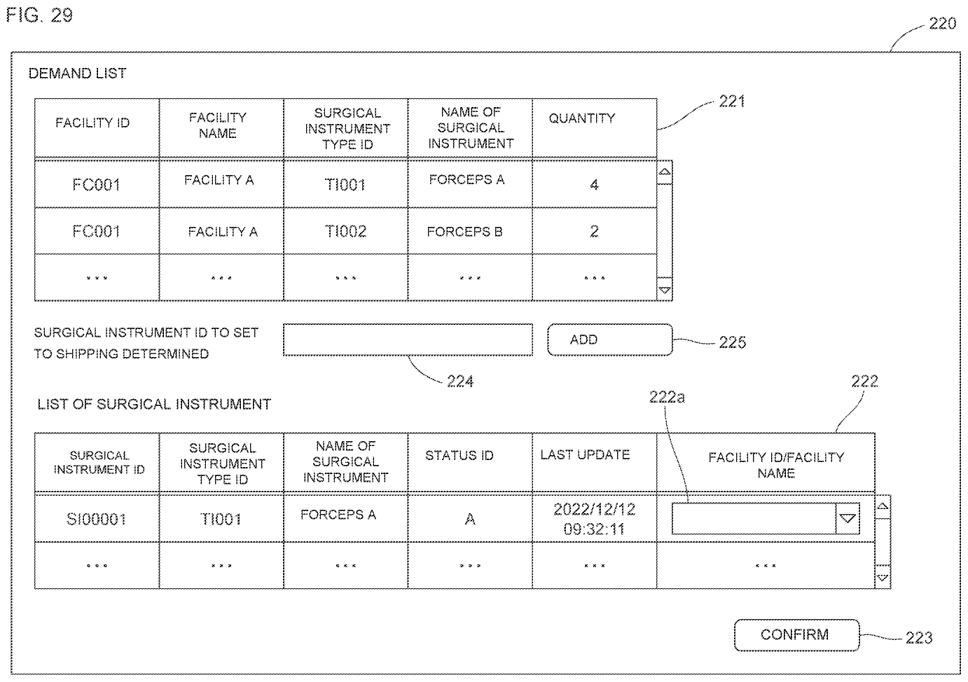

In FIG. 28, steps S461 and S462 are added in place of steps S402 and S412, respectively, compared to embodiment 1 shown in FIG. 21. In step S461, the controller 171 of the work terminal 21 sends a demand information request instruction. In step S462, the controller 141 of the information management system 40 generates demand information and transmits the generated demand information to the work terminal 21. In step S403, the controller 171 of the work terminal 21 receives the demand information from the information management system 40, and displays the shipping decision screen 220 shown in FIG. 29 on the display 173.

FIG. 29 schematically illustrates the shipping decision screen 220 displayed on the display 173 of the work terminal 21 in the device center 20 for this modification.

The shipping decision screen 220 in FIG. 29 has an additional text box 224 and an add button 225 compared to embodiment 1 shown in FIG. 22.

When the shipping decision screen 220 is displayed, the surgical instrument list 222 does not display information corresponding to the surgical instruments 30. In other words, initially, no surgical instruments 30 that can be selected as shipping targets are displayed.

The operator of the selection process at the device center 20 refers to the demand list 221 to determine the type and quantity of surgical instruments 30 that need to be delivered, and picks up the surgical instruments 30 to be shipped from the inventory. The operator then enters the surgical instrument ID of the picked-up surgical instruments 30 into the text box 211 and operates the add button 225. The controller 171 of the work terminal 21 displays the information about the surgical instrument ID entered in the text box 224 in the surgical instrument list 222. At this time, the controller 171 makes an inquiry to the information management system 40 based on the surgical instrument ID, obtains information about the relevant surgical instrument 30 from the information management system 40, and displays the obtained information in the surgical instrument list 222. If there are multiple surgical instruments 30 to be delivered, the operator performs the same operation to add information about the surgical instruments 30 to the surgical instrument list 222.

The operator then operates the pull-down menu 222a to select the shipping destination facility 10, as in embodiment 1, and then operates the confirm button 223. In this case, too, the surgical instruments 30 in the inventory are associated with the shipping destination facility 10, and the status of the surgical instruments 30 for which the shipping destination facility 10 is selected is updated to “shipping determined”.

In FIG. 29, when the add button 225 is operated, information about the surgical instrument ID entered in the text box 224 is obtained from the information management system 40, but information about the surgical instrument ID may not be obtained. In this case, for example, only the surgical instrument ID and the facility 10 items are displayed in the surgical instrument list 222.

### Modification 2 of Embodiment 1

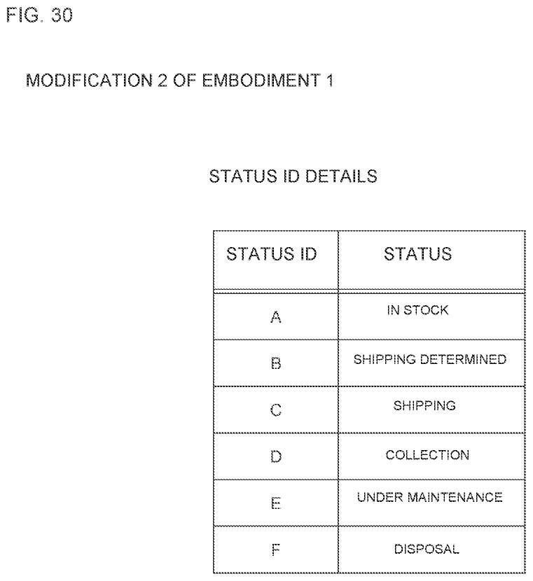

In embodiment 1, as shown in FIG. 12, the status ID value of the surgical instrument 30 could be set to any of the five types (A to E), but other status values may be set. For example, the status ID value may be further set to F (discard) as shown in FIG. 30.

As shown in FIG. 30, when disposal is provided as the status of the surgical instrument 30, the pull-down menu 212 of the update screen 210 shown in FIG. 19 is further configured to allow selection of F (disposal). The process for setting the status of the surgical instrument 30 to discard is the same as in FIG. 18.

This modification prevents the surgical instruments 30 set for disposal from being accidentally reused afterwards.

### Embodiment 2

In embodiment 2, compared to embodiment 1, the database DB stores information regarding the schedule of surgeries to be performed at each of the 10 facilities.

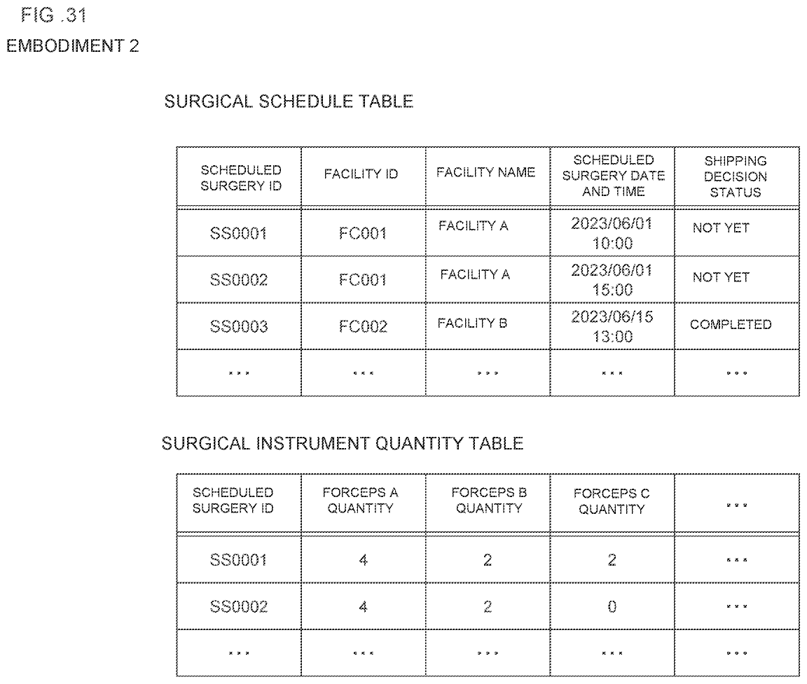

FIG. 31 schematically shows the structure of the surgery schedule table and the quantity of surgical instruments table stored in the database DB, according to embodiment 2.

As shown in the top row of FIG. 31, the surgery schedule table contains items for scheduled surgery ID, facility ID, facility name, scheduled surgery date and time, and shipping decision status. The scheduled surgery ID item indicates an ID that individually identifies the surgery to be performed at each of the 10 facilities. The scheduled surgery date and time item indicates the date and time of the surgery indicated by the scheduled surgery ID. The shipping decision status item indicates whether or not all surgical instruments 30 required for the surgery indicated by the scheduled surgery ID have been decided to be delivered.

As shown in the lower part of FIG. 31, the quantity of surgical instruments table contains items for the scheduled surgery ID and the quantity of surgical instruments 30 corresponding to each of the types of all surgical instruments 30. The item of the surgery schedule ID corresponds to the surgery schedule ID in the surgery schedule information table. The item of the quantity of surgical instruments 30 for each type indicates the quantity of surgical instruments 30 of that type that are scheduled to be used in the surgery corresponding to the scheduled surgery ID.

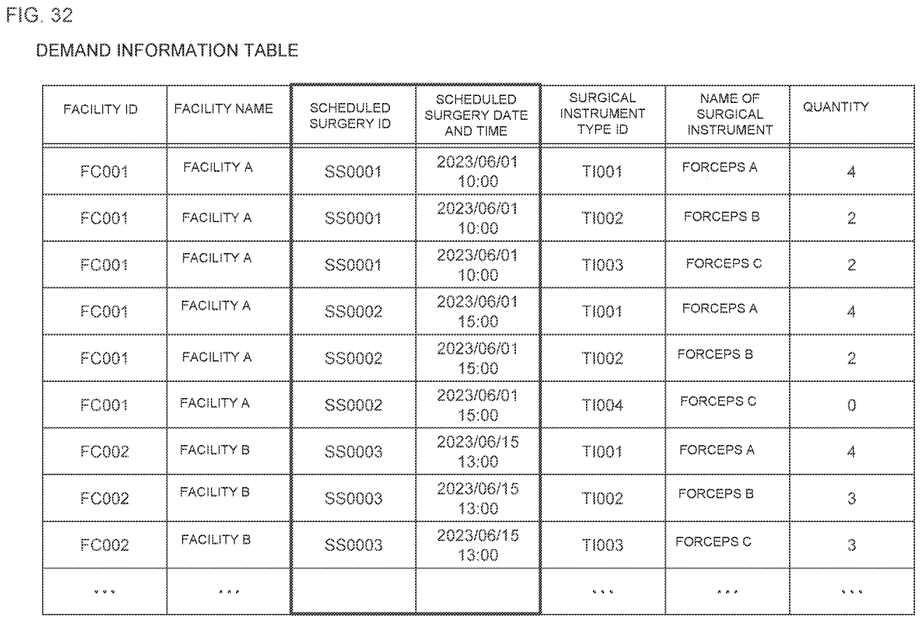

FIG. 32 schematically shows the composition of the demand information table stored in the database DB for embodiment 2.

Compared to embodiment 1 shown in FIG. 13, the demand information table in this modified example includes additional items for the scheduled surgery ID and the scheduled surgery date and time.

When the controller 141 of the information management system 40 receives the surgery schedule information (see FIG. 3) transmitted from the work terminal 13 of the facility 10, it adds records to the scheduled surgery table and the quantity of surgical instruments table shown in FIG. 31 based on the scheduled surgery information. The controller 141 also adds records to the demand information table based on the information added to the scheduled surgery table and the quantity of surgical instruments table.

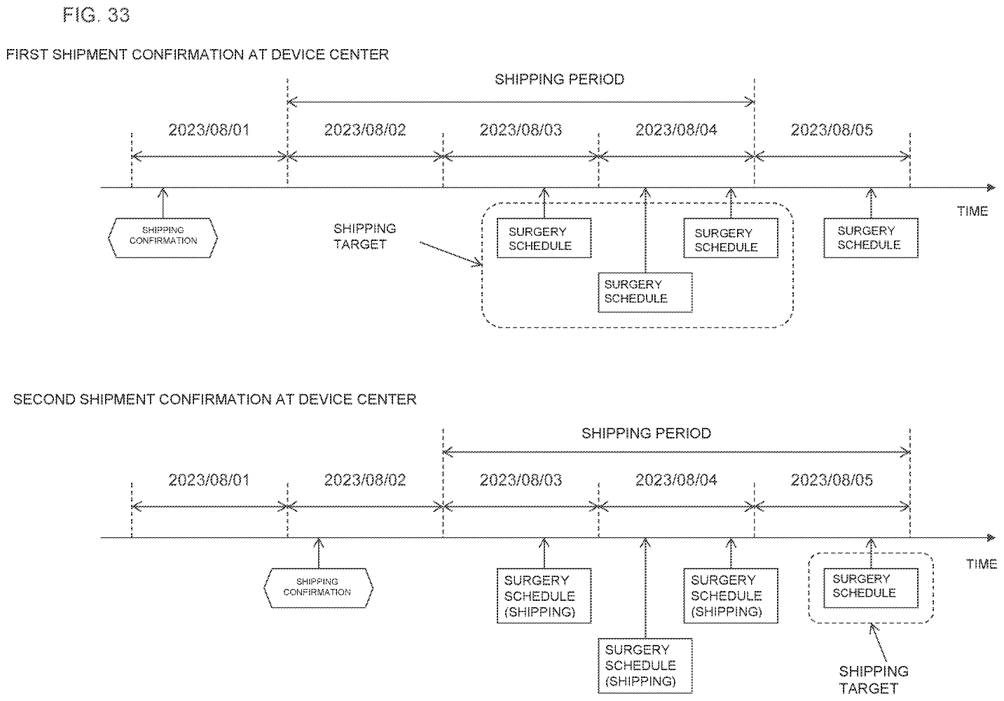

FIG. 33 is a schematic diagram illustrating shipment confirmation at the device center 20.

In the selection process of the device center 20, the operator confirms the shipment of the required surgical instruments 30 at each facility 10 at predetermined time intervals using the work terminal 21. For example, shipment confirmation is performed at 8:00 a.m. daily. The upper part of FIG. 33 shows an example of a shipment confirmation on Aug. 1, 2023, and the lower part of FIG. 33 shows an example of a shipment confirmation on the following day.

As shown in the top panel of FIG. 33, when the shipment confirmation is made on Aug. 1, 2023, the target period for delivery is determined. The target period for delivery is set to be the period of time from the shipping decision is made on the day of the shipment confirmation, which the surgical instruments 30 arrive at the destination facility 10 no too much earlier than the scheduled surgery date but no later than the scheduled surgery date. For example, if it takes up to three days for the surgical instruments 30 to arrive at the facility 10 after the shipping decision is made, the target shipping period is set to three days from the day after the shipment confirmation.

When the controller 141 of the information management system 40 receives a shipment confirmation from the work terminal 21 of the device center 20, it sets the target period for delivery as August 2 to August 4, refers to the surgical schedule table, and sets the surgical schedules set for the target period for delivery. Then, the controller 141 refers to the quantity of surgical instruments table to obtain the type and quantity of surgical instruments 30 required for the surgical schedule to be delivered, and sends the request information including the obtained surgical schedule, the type and the quantity of the surgical instruments 30 to the work terminal 21. The operator of the device center 20 then proceeds with the shipping procedures of the surgical instruments 30 required for the three scheduled surgeries on August 3 to August 4 based on the request information from the information management system 40.

As shown in the lower part of FIG. 33, the surgical instruments 30 required for the shipping target period on the previous day are already in shipping status. As on the previous day, when the shipment is confirmed on Aug. 2, 2023, the controller 141 of the information management system 40 sets the delivery target period to Aug. 3 to Aug. 5, 2023, and the surgical schedule set for the delivery target period. The operator of Device Center 20 then proceeds with the shipping procedure of the surgical instruments 30 required for the one scheduled surgery on August 5, based on the request information from the information management system 40.

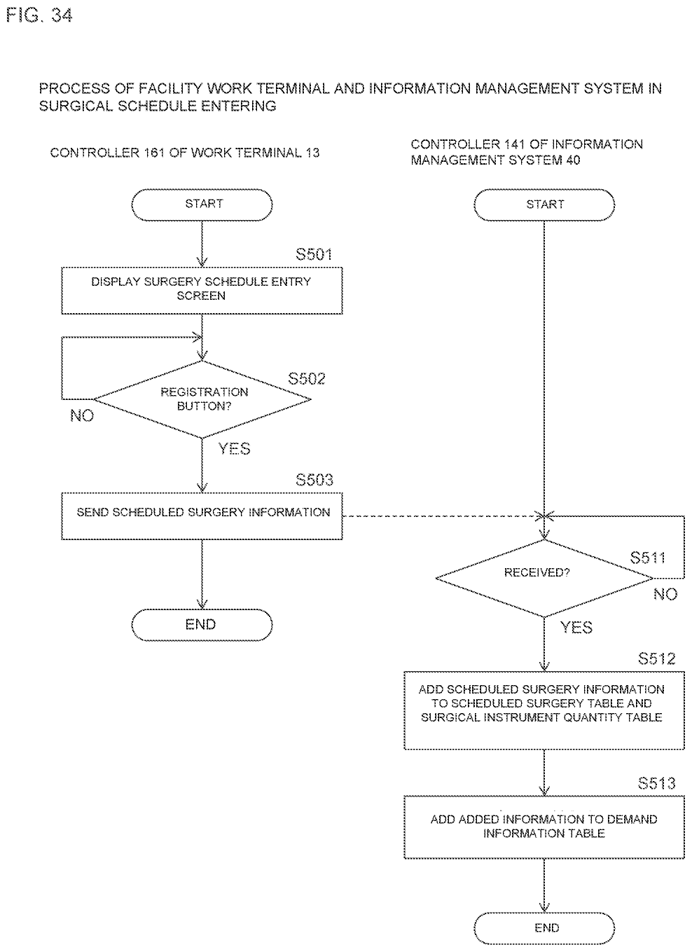

FIG. 34 is a flowchart showing the process of the work terminal 13 of the facility 10 and the information management system 40 when entering the surgery schedule.

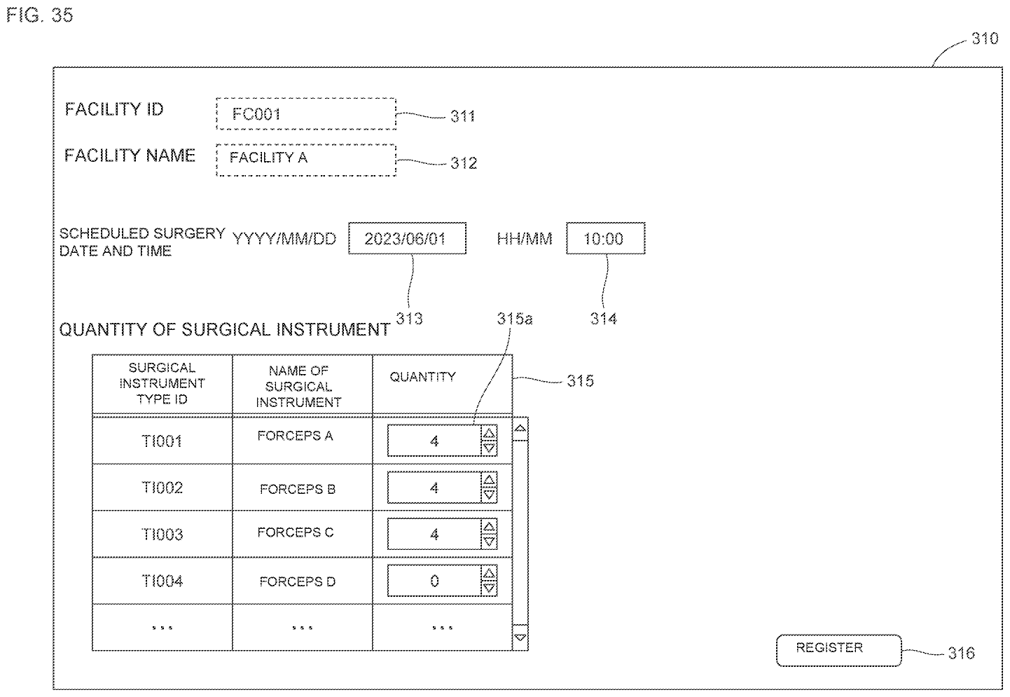

The operator in charge of entering the surgery schedule at the facility 10 operates the input unit 164 of the work terminal 13 and enters an instruction to display the surgery schedule input screen 310 (see FIG. 35) for entering the surgery schedule. When the controller 161 of the work terminal 13 receives the display instruction, it displays the surgery schedule input screen 310 on the display 163 in step S501.

FIG. 35 schematically depicts the surgery schedule input screen 310 displayed on the display 163 of the work terminal 13 in the facility 10.

The surgery schedule entry screen 310 has display areas 311 and 312, text boxes 313 and 314, a list of the quantity of surgical instruments 315, and a registration button 316.

Display areas 311 and 312 display the facility ID and name of the facility 10 in question, which are stored in the memory 162 of the work terminal 13 in advance. Text boxes 313 and 314 are text boxes for entering the scheduled surgery date and scheduled surgery time, respectively.

The list of the quantity of surgical instruments 315 includes items for the type IDs of the surgical instruments 30, the surgical instrument names, and the quantity of the surgical instruments 30. The list of the quantity of surgical instruments 315 displays the type IDs and surgical instrument names corresponding to the surgical instruments 30 of all type IDs, row by row. The item for the quantity of surgical instruments 30 has a text box 315a with an attached button to increase or decrease the number.

The operator of facility 10 uses the input unit 164 of work terminal 13 to enter the scheduled surgery date and time into text boxes 313 and 314, and the quantity of surgical instruments 30 of each type needed for that surgery into text box 315a. When the operator has completed the input, he or she operates the registration button 316.

Returning to FIG. 34, when the registration button 316 is operated (step S502: YES), in step S503, the controller 161 of the work terminal 13 sends the scheduled surgery information, including the facility ID and facility name entered in the display areas 311 and 312, the scheduled surgery date and time entered in the text boxes 313 and 314, the surgery schedule information including the facility ID and facility name entered in display areas 311 and 312, the scheduled surgery date and time entered in text boxes 313 and 314, and the quantity of the surgical instruments 30 per type entered in text box 315a is sent to the information management system 40.

When the controller 141 of the information management system 40 receives the surgery schedule information from the work terminal 13 (step S511: YES), it adds the surgery schedule information to the surgery schedule table and the quantity of surgical instruments table shown in FIG. 31 in step S512. Specifically, the controller 141 adds one record to the surgery schedule table, consisting of the automatically assigned surgery schedule ID, the facility ID, facility name, and scheduled surgery date and time included in the surgery schedule information. At this time, the shipping decision status item is initially set to “Not yet”. The controller 141 also adds one record to the quantity of surgical instruments table, consisting of the scheduled surgery ID and the quantity of surgical instruments 30 for each type included in the scheduled surgery information.

In step S513, the controller 141 of the information management system 40 adds the record containing the information added to the database DB in step S513 to the demand information table shown in FIG. 32.

In embodiment 2, the same process as in embodiment 1 shown in FIG. 21 is performed based on the demand information table updated as described above. However, in embodiment 2, in step S412 of FIG. 21, the controller 141 of the information management system 40 sets the delivery target period as shown in FIG. 33, and refers to the surgical schedule table (see FIG. 31) to obtain a surgical schedule ID whose shipping decision status is still “Not yet”. The controller 141 then generates demand information including the type and quantity of the surgical instruments 30 required for the surgical schedules for the delivery target period that have not yet been determined for shipping, based on the acquired surgical schedule IDs and the demand information table (see FIG. 32).

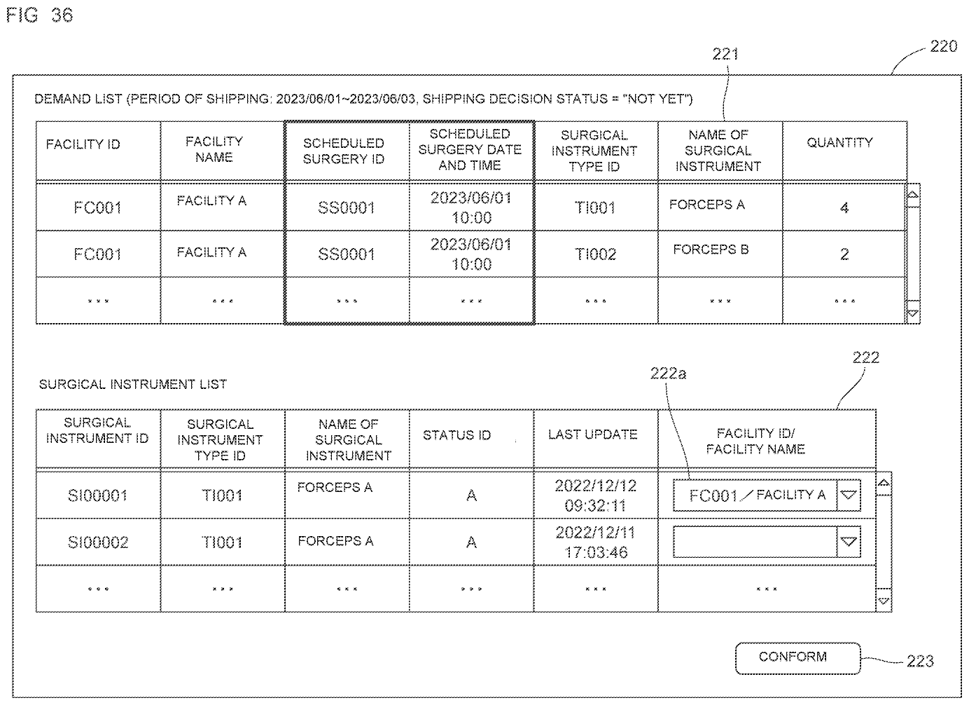

In embodiment 2, the shipping decision screen 220 displayed on the work terminal 21 in step S403 of FIG. 21 is configured as shown in FIG. 36. Compared to embodiment 1 shown in FIG. 22, the shipping decision screen 220 in FIG. 36 has an additional item of scheduled surgery ID and scheduled surgery date and time in the demand list 221. In embodiment 2, in the demand list 221, the type and quantity of surgical instruments 30 required for the scheduled surgery for the delivery target period are displayed based on the demand information transmitted from the information management system 40.

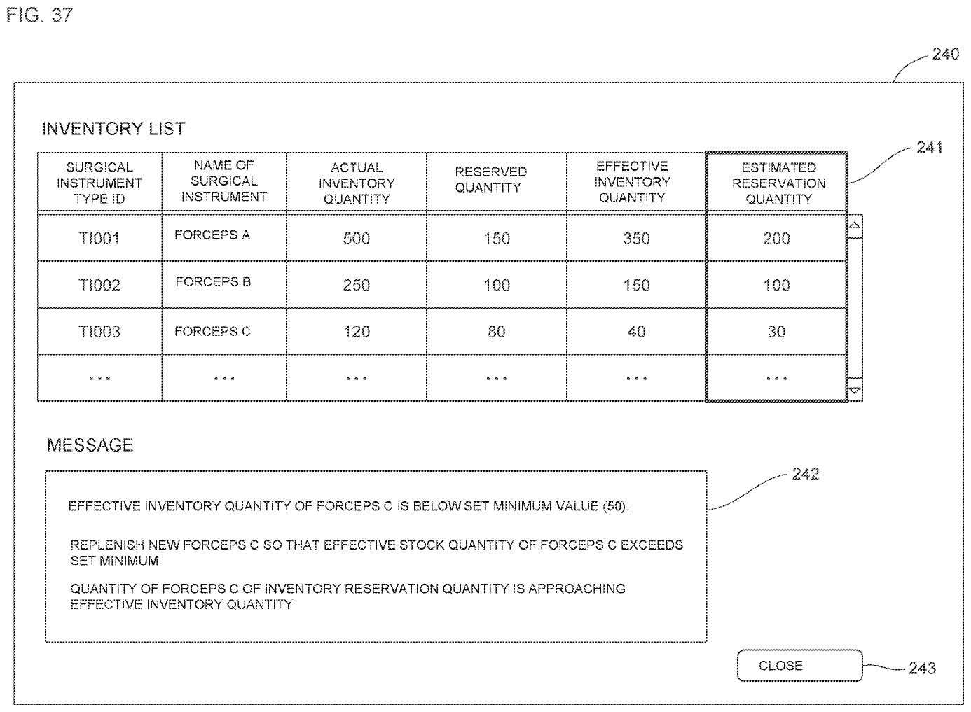

FIG. 37 schematically illustrates the inventory screen 240 displayed on the display 173 of the work terminal 21 in the device center 20, according to embodiment 2.

The inventory screen 240 of embodiment 2, compared to embodiment 1 shown in FIG. 27, has an additional item in the inventory count list 241, the estimated reservation quantity.

The item estimated reservation quantity indicates the quantity of the surgical instruments 30 required for all scheduled surgeries that have not yet been determined to be shipped (i.e., not yet reserved). The estimated reservation quantity is the quantity of surgical instruments 30 that will be shipped from the inventory in the future, together with the quantity of items to be reserved. If the sum of the quantity of reservations and the estimated reservation quantity exceeds the actual inventory, or in other words, if the estimated reservation quantity exceeds the number of available inventory, a situation may occur in the future where there will not be enough surgical instruments 30 available. For this reason, a warning message is displayed in the message area 242, indicating that the estimated reservation quantity of forceps C is approaching the number of effective inventory.

By referring to the inventory count list 241 and message area 242, the operator of device center 20 can refer to the quantity of surgical instruments 30 that have not yet been determined to be shipped but will be needed for future surgeries (the estimated reservation quantity) and understand that the inventory of surgical instruments 30 may be insufficient. The system can identify the possibility of a shortage of surgical instruments 30 in stock.

In embodiment 2, the inventory screen 240 is displayed by the same process as in embodiment 1 shown in FIG. 26. In this case, in step S452 of FIG. 26, based on the surgery schedule table (see FIG. 31) and the demand information table (see FIG. 32), the controller 141 of the information management system 40 obtains, for each type of surgical instrument 30, the quantity of surgical instruments 30 that have not yet been determined for shipping and are needed for future operations as the estimated reservation quantity. The controller 141 then transmits the information on the number of inventory items including the planned quantity of items to the work terminal 21.

### <Effectiveness of the Method for Managing Surgical Instruments and Information Management System According to Embodiment 2>

As shown in the demand list 221 in FIG. 36, the demand information transmitted from the information management system 40 to the work terminal 21 includes surgery schedule information regarding scheduled surgeries using the surgical robot system 11 at the facility 10.

According to this configuration, the type and quantity of surgical instruments 30 that need to be delivered to a facility 10, such as a medical facility, can be provided to the facility 10 based on the surgical schedule.

In embodiment 2, a list of the quantity of surgical instruments 315 for entering the quantity of surgical instruments 30 was placed on the surgery schedule input screen 310 in FIG. 35, but the list of the quantity of surgical instruments 315 may be omitted. In this case, the type and quantity of surgical instruments 30 required for one surgery are stored in the database DB, and the controller 141 of the information management system 40 sends the demand information including the type and quantity of surgical instruments 30 stored in the database DB in advance to the work terminal 21 of the device center 20.

According to this configuration, the operator of facility 10 does not need to enter the type and quantity of surgical instruments 30 required based on the surgery schedule. This further reduces the burden on the facility 10.

As shown in the demand list 221 in FIG. 36, the surgery schedule information contained by the demand information includes information about the scheduled surgery date on which the surgery will be performed using the surgical robot system 11.

This configuration allows the operator of the device center 20 to manage the shipping of the surgical instruments 30 in time for the scheduled surgery date.

In embodiment 2, the demand list 221 in FIG. 36 displays only information about the surgeries scheduled to be performed during the shipping target period. As explained with reference to FIG. 33, the target period for shipping is the period during which the surgical instruments 30 arrive at the destination facility 10 no too much earlier than the scheduled surgery date and no later than the scheduled surgery date after the shipping is decided on the same day of the shipment confirmation. This allows the operator of the device center 20 to smoothly deliver the surgical instruments 30 to the destination facility 10 at the appropriate time for the scheduled surgery date.

As shown in FIG. 36, demand list 221, demand information is provided at times corresponding to the scheduled surgery date.

According to this configuration, demand information can be provided to device center 20, etc., which is responsible for managing the shipping of surgical instruments 30, at a time corresponding to the scheduled surgery date. Specifically, the demand list 221 in FIG. 36 displays information on surgeries scheduled to be performed during the period covered by the shipping corresponding to the scheduled surgery date and time. This allows smooth shipping of the appropriate type and quantity of surgical instruments 30 according to the scheduled surgery date.

### Embodiment 3

In embodiment 2, the demand information table in FIG. 32 was updated based on the scheduled surgery table and the quantity of surgical instruments table in FIG. 31. In contrast, in embodiment 3, the surgery schedule table further includes other items and a combination table is stored in the database DB instead of the quantity of surgical instruments table.

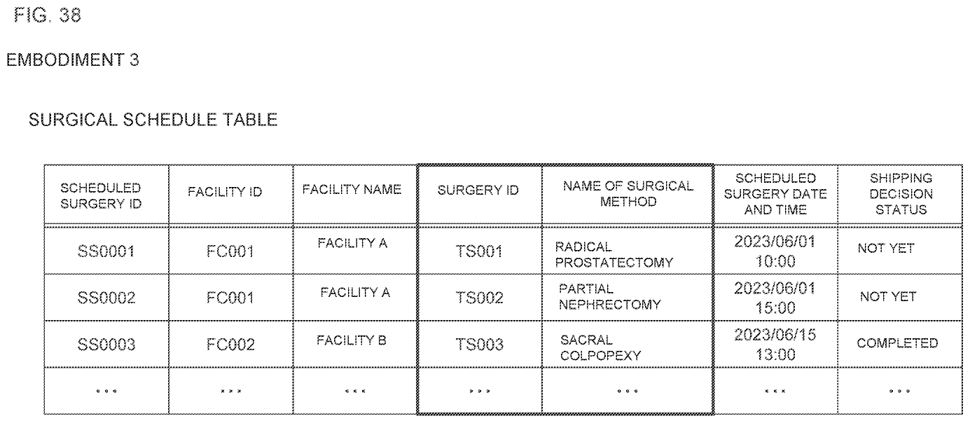

FIG. 38 schematically shows the structure of the surgery schedule table stored in the database DB for embodiment 3.

Compared to embodiment 2 shown in FIG. 31, the scheduled surgery table of embodiment 3 contains additional items for a surgical procedure ID and a surgical procedure name. The operation ID item indicates an ID that can individually identify the operation to be performed. The operation name item indicates the name of the operation indicated by the operation ID. In the example shown in FIG. 38, the surgery with the scheduled surgery ID of SS0001 is the surgery with the procedure ID of TS001 and the procedure name of radical prostatectomy. The surgery with the scheduled operation ID of SS0002 is a partial nephrectomy surgery with the operation ID of TS002. The surgery with the scheduled operation ID of SS0003 is a sacral colpopexy surgery with the operation ID of TS003.

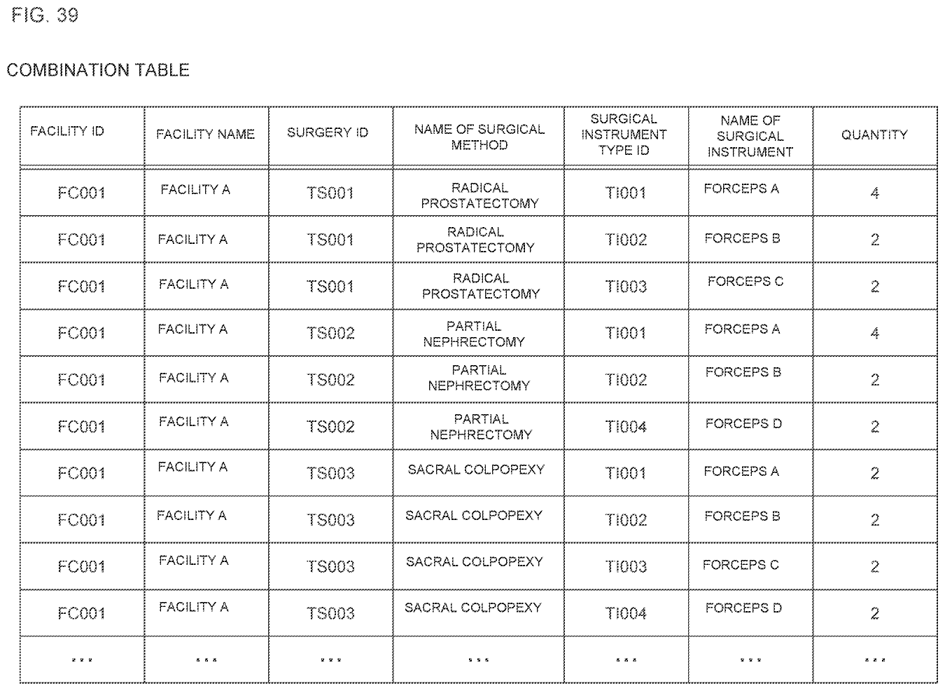

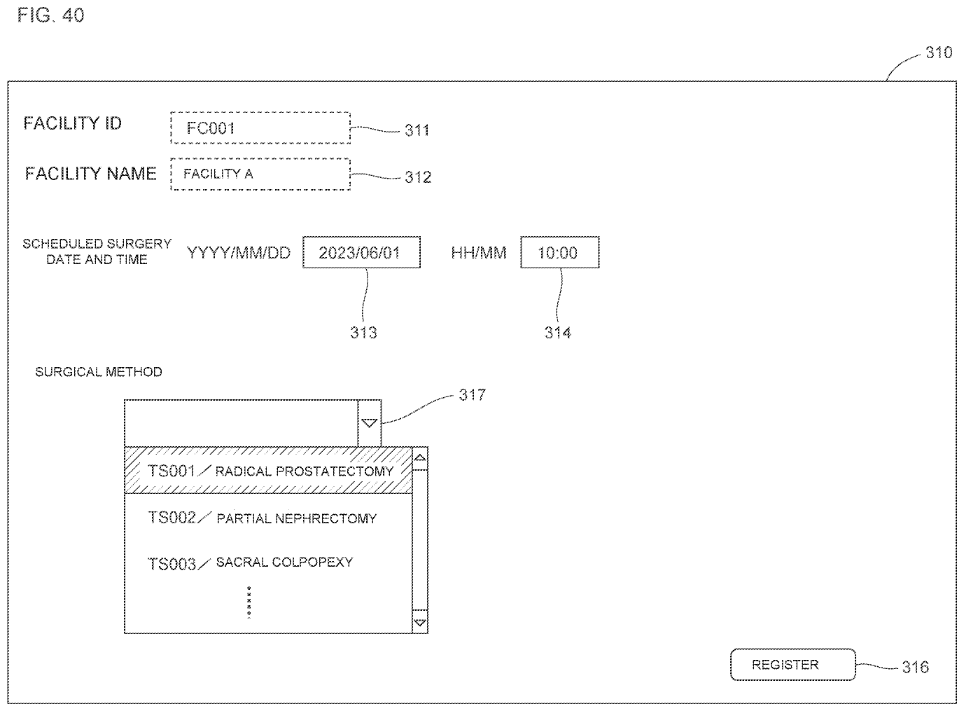

FIG. 39 schematically shows the structure of the combination table stored in the database DB according to embodiment 3.

The combination table contains the following items: facility ID, facility name, surgical procedure ID, surgical procedure name, type ID of surgical instruments 30, surgical instrument name, and quantity of surgical instruments 30. In other words, in embodiment 3, the type and quantity of surgical instruments 30 required at each facility 10 are determined according to the surgical procedure.

FIG. 40 schematically illustrates the configuration of the surgery schedule input screen 310 displayed on the display 163 of the work terminal 13 in the facility 10, in accordance with embodiment 3.

Compared to embodiment 2 shown in FIG. 35, embodiment 3's surgery schedule entry screen 310 has a surgical procedure pull-down menu 317 in place of the list of the quantity of surgical instruments 315.

The surgical procedure pull-down menu 317 is configured to allow selection of all surgical procedure ID and procedure name combinations. The operator of the facility 10 enters the scheduled surgery date and time into text boxes 313 and 314, and operates the surgery procedure pull-down menu 317 to select the operation type ID and name for that surgery. When the operator has completed the input, he or she operates the registration button 316.

In embodiment 3, the same process as in embodiment 2 shown in FIG. 34 is executed. However, in embodiment 3, in step S503 of FIG. 34, the controller 161 of the work terminal 13 transmits the surgery schedule information including the surgery ID and the surgery name selected in the surgery procedure pull-down menu 317 shown in FIG. 40. In step S512 of FIG. 34, the controller 141 of the information management system 40 adds one record including the automatically assigned surgery schedule ID, the facility ID, facility name, and scheduled surgery date and time included in the surgery schedule information, plus the surgery ID and name to the surgery schedule table in FIG. 38. In step S513 of FIG. 34, the controller 141 adds a record to the demand information table similar to that of embodiment 2 shown in FIG. 32 based on the record containing the information added to the database DB in step S513 and the combination table shown in FIG. 39.

Thus, in embodiment 3, instead of entering the type and quantity of surgical instruments 30 to be used in a scheduled surgery, the operator of facility 10 enters the procedure ID and the name of the procedure. This allows the type and quantity of surgical instruments 30 required for each scheduled surgery to be obtained from the demand information table (see FIG. 32), as in embodiment 2.

### <Effectiveness of the Method for Managing Surgical Instruments and Information Management System According to Embodiment 3>

Surgery schedule information includes a procedure ID and a procedure name item (content information regarding the contents of the surgery).

According to this configuration, the surgical instruments 30 required for a surgery can be prepared and delivered flexibly, even when the type and quantity of surgical instruments 30 required differ according to the type of surgery and other details of the surgery, such as the surgical technique.

A record is added to the demand information table based on the items of the procedure ID and procedure name (content information about the content of the surgery) and the combination table in FIG. 39 (information about the combination of multiple surgical instruments 30 that need to be delivered to the facility 10).

According to this configuration, since demand information is automatically generated based on the surgical procedure and the combination of the surgical instruments 30, it is not necessary for the person in charge at the facility 10 to combine the surgical instruments 30 required for the procedure, thus reducing the burden on the facility 10 side.

### Example of Modification of Embodiment 3

In embodiment 3, the surgery schedule table and combination table shown in FIGS. 38 and 39 may include other items together with or instead of the items of procedure ID and procedure name. In the modification of embodiment 3 shown below, the surgery schedule table and the combination table further include an item for the surgeon.

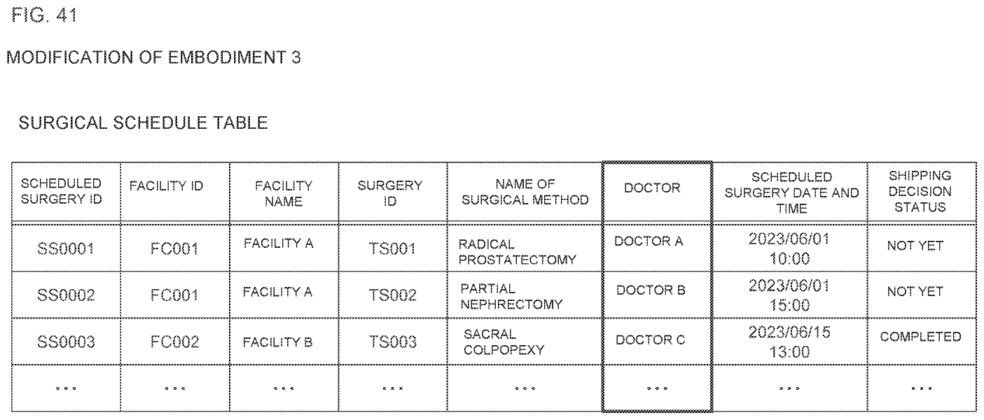

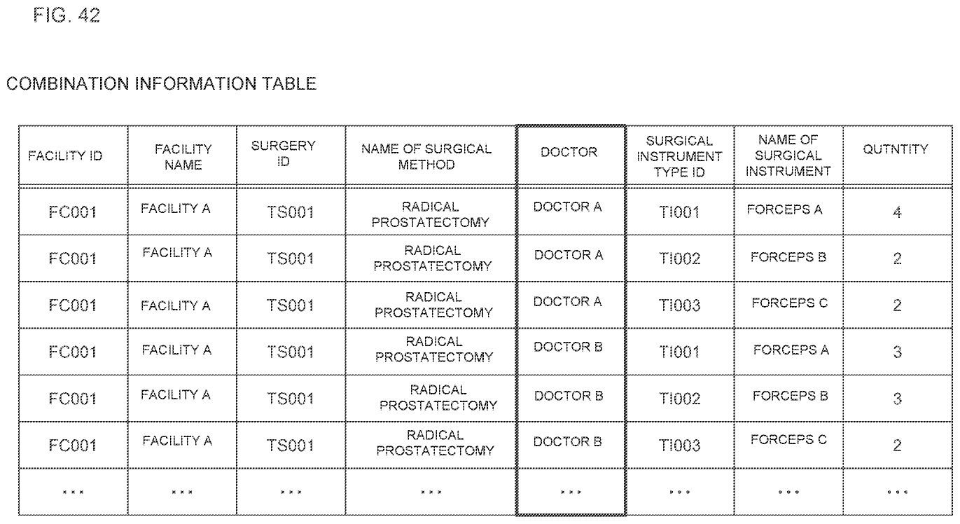

FIGS. 41 and 42 schematically illustrate the structure of the scheduled surgery table and the combination table for this modification.

Both the scheduled surgery table and the combination table in this modified example include an additional item for the surgeon, compared to embodiment 3 shown in FIGS. 38 and 39. The item of the surgeon indicates the name of the surgeon, such as the doctor who will perform the scheduled surgery. The surgeon's item should be configured so that the surgeon can be identified individually. The surgeon's item may be, for example, a surgeon ID assigned to the surgeon, or it may be the surgeon's name with other information added to the surgeon's item. In the example of the combination table shown in FIG. 42, the facility ID, facility name, procedure ID and procedure name are the same, but the quantity of the surgical instruments 30 required for each other is different when the surgeon is Doctor A and Doctor B.

In this modified example, the type and quantity of the surgical instruments 30 required for each surgical schedule can still be obtained based on the surgical schedule table and the combination table.

### <Effects of the Method of Managing Surgical Instruments and Information Management System Based on a Modification of Embodiment 3>

Compared to embodiment 3, the surgery schedule information further includes a surgeon item (surgeon information regarding the surgeon operating the surgical robot system 11).

According to this configuration, the surgical instruments 30 required for a surgery can be prepared and delivered flexibly, even when the type and the quantity of the surgical instruments 30 required differ according to the details of the surgery and the surgeon. In addition, since the item of the surgeon is added to embodiment 3, the type and quantity of surgical instruments 30 can be determined in detail according to the surgery.

In embodiment 3, the surgery schedule table and the combination table may include an item for the surgeon instead of an item for the procedure ID and procedure name. In this case, the surgical instruments 30 required for the surgery can still be prepared and delivered flexibly, even if the type and quantity of surgical instruments 30 required differ depending on the surgeon.

Based on the items of the procedure ID and procedure name (content information about the content of the surgery), the item of the surgeon (surgeon information about the surgeon operating the surgical robot system 11), and the combination table in FIG. 41 (information about the combination of multiple surgical instruments 30 that need to be delivered to the facility 10), a record in the demand information table is added to the demand information table.

According to this configuration, since demand information is automatically generated based on the surgical content and surgeon and the combination of surgical instruments 30, there is no need for the operator of facility 10 to combine the surgical instruments 30 necessary for the surgery, thereby reducing the burden on the facility 10 side. In addition, since the item of the surgeon is used compared to embodiment 3, the demand information can be determined in detail.

In embodiment 3, a record may be added to the demand information table based on the item of the surgeon (surgeon information regarding the surgeon operating the surgical robot system 11) and the combination table in FIG. 41 (information regarding the combination of multiple surgical instruments 30 that need to be delivered to the facility 10). In this case, the demand information is automatically generated based on the surgeon and the combination of surgical instruments 30, thus reducing the burden on the facility 10.

### Embodiment 4

In embodiment 4, compared to embodiment 2, a utilization result table is stored in the database DB indicating which type and quantity of surgical instruments 30 were used at which facilities 10 in the past.

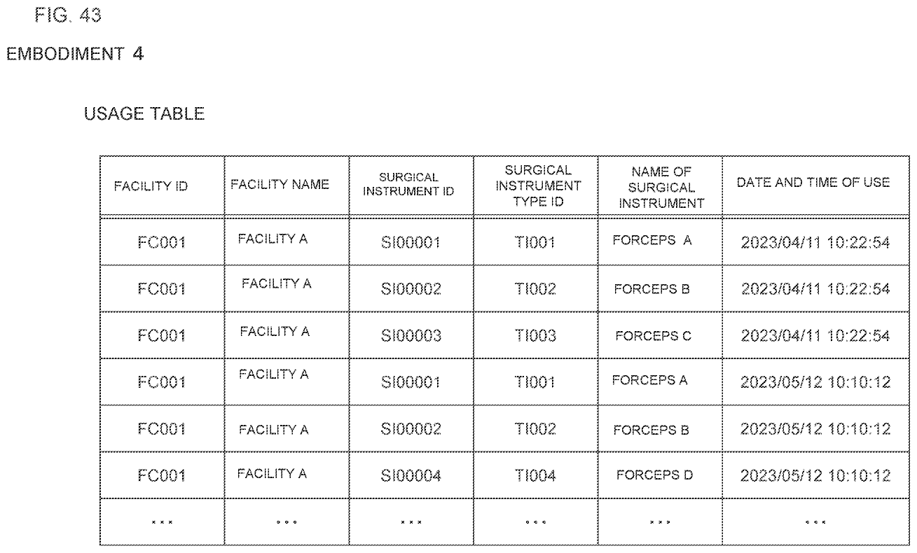

FIG. 43 schematically illustrates the structure of the utilization result table for embodiment 4.

The utilization table contains items for facility ID, facility name, surgical instrument ID, surgical instrument 30 type ID, surgical instrument name, and date and time of use. The date of use item indicates the date and time of the surgical instruments 30 used at the facility 10.

When a surgical instrument 30 is used and the status of the surgical instrument 30 is set to collection, the controller 141 of the information management system 40 adds a record to the usage table containing the facility ID and name of the facility where the surgical instrument 30 was used, the surgical instrument ID, type ID and surgical instrument name of the surgical instrument 30, the current date and time corresponding to the date of use of the surgical instrument 30. The records including the facility ID and name of the facility where the surgical instrument 30 was used, the surgical instrument ID, type ID and name of the surgical instrument 30, and the current date and time corresponding to the date and time of use of the surgical instrument 30 are added to the usage table.

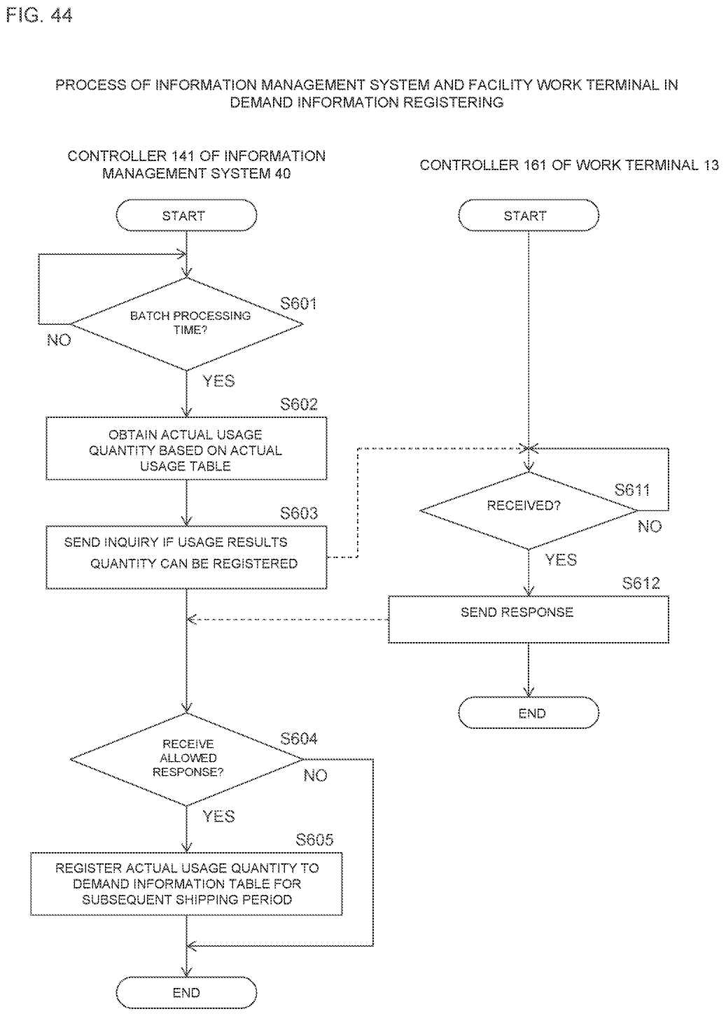

FIG. 44 is a flowchart showing the processing of the information management system 40 and work terminal 13 when registering demand information, according to embodiment 4.

When the current time becomes the batch processing time (step S601: YES), the controller 141 of the information management system 40 starts processing from step S602 onward. The batch processing time is set so that the processing of steps S602 to S605 will be completed by the time of the shipment confirmation (see FIG. 33) at the device center 20, for example, at 4:00 pm on the day before the shipment confirmation at the device center 20. The interval between batch processing is, for example, one month.

In step S602, the controller 141 obtains the number of actual use of the surgical instruments 30 expected to be used in the time width of the shipping target period (see FIG. 33) for each facility ID and type ID of the surgical instruments 30 based on the table of actual use shown in FIG. 43. The quantity of surgical instruments 30 expected to be used in the time span of the target shipping period is, for example, the average number of times the surgical instruments 30 were used in the time span of the target shipping period, based on the total actual usage for the past year in the actual usage table. In step S603, the controller 141 sends a query to each of the 10 facilities asking whether or not to register the number of actual uses in the demand information table similar to FIG. 13.

When the controller 161 of the work terminal 13 of the facility 10 receives the inquiry sent in step S603 (step S611: YES), the operator of the facility 10 sends a response to the information management system 40 in step S612, indicating whether the actual usage count is acceptable or not. Specifically, if the operator of facility 10 determines that there is no problem with the received actual usage count, the operator inputs the answer to the effect that the registration of the actual usage count in the demand information table is permitted to the work terminal 13. On the other hand, if the operator of facility 10 determines that there is a problem with the received number of actual usage, the operator inputs a response to the work terminal 13 to the effect that the registration of the number of actual usage to the demand information table is not permitted. The controller 161 then transmits the entered response to the information management system 40.

When the controller 141 of the information management system 40 receives a response of permission from the work terminal 13 (step S604: YES), it registers the number of actual usage results obtained in step S602 in the subsequent shipping target period (see FIG. 33) in the demand information table similar to FIG. 13. In this way, the type and quantity of the surgical instruments 30 required at each facility 10 are automatically registered in the demand information table without the operator of the facility 10 having to input the surgery schedule.

On the other hand, if the controller 141 receives a non-permitted response from the work terminal 13 (step S604: NO), step S605 is omitted. In this case, the operator of the facility 10 enters, for example, via the work terminal 13, the type and the quantity of the surgical instruments 30 planned to be used at the facility 10. The controller 141 of the information management system 40 registers the type and quantity of surgical instruments 30 received from the work terminal 13 in a demand information table similar to FIG. 13.

In embodiment 4, the same process as in FIG. 21 is performed. In this case, the demand information based on the demand information table in which the quantity of actual usage is registered as described above is displayed in the demand list 221 of the shipping decision screen 220 as in FIG. 22.

In the process of FIG. 44, the controller 141 of the information management system 40 inquired to the facility 10 whether or not the number of actual usage records could be registered, but the inquiry may be omitted and the registration may be performed automatically. In this case, the controller 141 notifies the corresponding facility 10 of the automatic registration.

### <Effectiveness of the Method for Managing Surgical Instruments and Information Management System According to Embodiment 4>

The information in the demand information table (demand information) is generated based on the information in the utilization result table (utilization result information regarding the surgical instruments 30 at the facility 10) (steps S602 and S605 in FIG. 44).

According to this configuration, the operator of the device center 20 can determine the type and the quantity of surgical instruments 30 needed at the facility 10 without the operator in charge of inputting the type and quantity of surgical instruments 30 needed for the scheduled surgery.

### Embodiment 5

In embodiment 3, fixed combination information was set in advance in the combination table shown in FIG. 39. In contrast, in embodiment 5, compared to embodiment 3, the combination information in the combination table is updated based on the information in the utilization result table shown in FIG. 43.

FIG. 45 schematically illustrates the structure of the utilization result table for embodiment 5.

Compared to embodiment 4 shown in FIG. 43, the utilization result table in embodiment 5 contains additional items for the procedure ID and the name of the procedure.

In embodiment 5, when the surgical instrument 30 is used and the status of the surgical instrument 30 is set to collection, the controller 141 of the information management system 40 adds a record to the usage performance table. The record includes the facility ID and facility name where the surgical instrument 30 was used, the surgical procedure ID and surgical procedure name where the surgical instrument 30 was used, the surgical instrument ID, type ID, and surgical instrument name of the surgical instrument 30, and the current date and time corresponding to the date and time the surgical instrument 30 was used

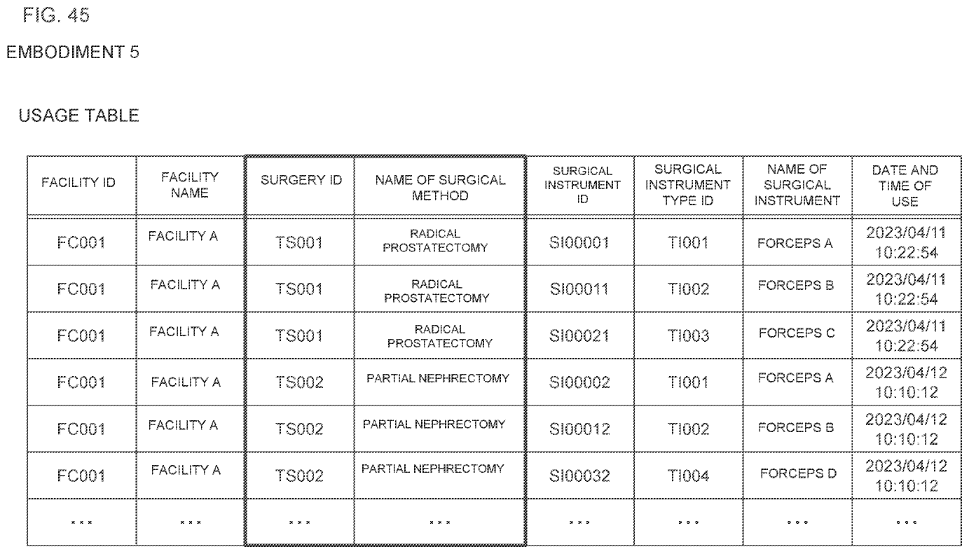

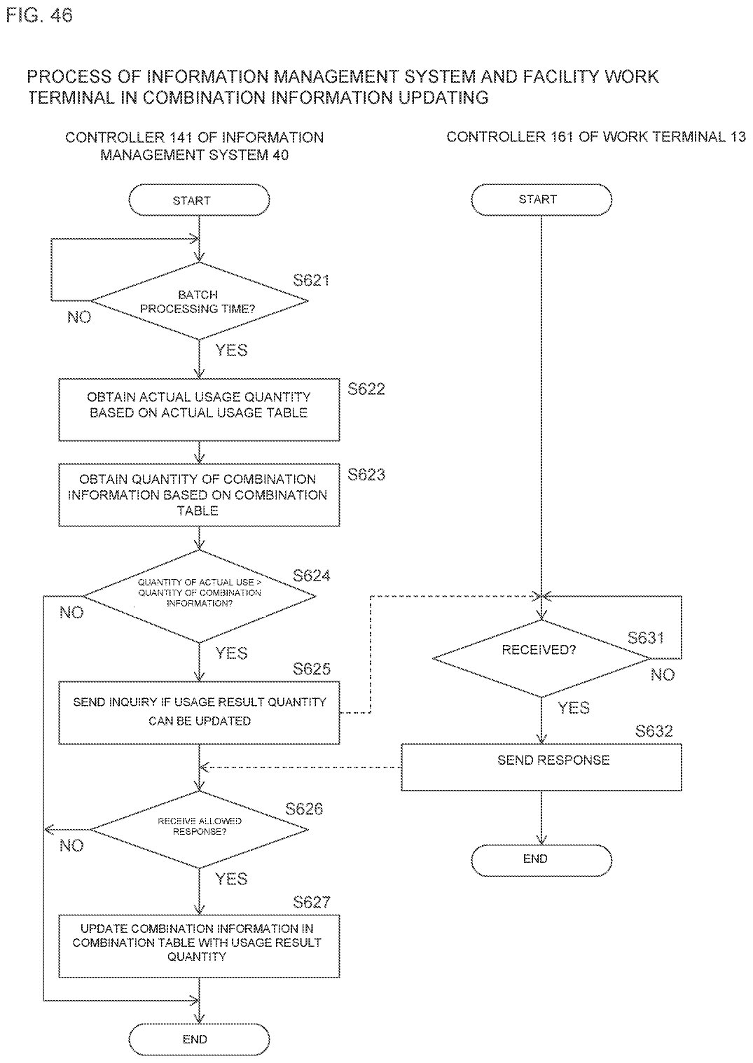

FIG. 46 is a flowchart showing the process of the information management system 40 and work terminal 13 when updating combination information.

When the current time becomes the batch processing time (step S621: YES), the controller 141 of the information management system 40 starts processing from step S622 onward. The batch processing time is set so that the processing of steps S622 to S627 will be completed before the shipment confirmation (see FIG. 33) at the device center 20, for example, at 4:00 p.m. on the day before the shipment confirmation at the device center 20. The interval between batch processing is, for example, one month.

In step S622, the controller 141 of the information management system 40 obtains the number of actual use of the surgical instruments 30 that are expected to be used in the time width of the shipping target period (see FIG. 33) for each facility ID, procedure ID and type ID of the surgical instruments 30 based on the utilization result table shown in FIG. 45. The quantity of the surgical instruments 30 expected to be used in the time span of the target shipping period is, for example, the average number of times the surgical instruments 30 were used in the time span of the target shipping period based on all the past one-year utilization records in the utilization result table.

In step S623, the controller 141 obtains the number of combination information, which is the number of usage of the surgical instruments 30 in the time range of the shipping target period, for each facility ID, procedure ID and type ID of the surgical instruments 30 based on the combination table shown in FIG. 39. In this case, the combination table defines the number of usage of surgical instruments 30 in the shipping target period in the number field.

In step S624, the controller 141 determines whether the number of actual utilization results obtained in step S622 is greater than the number of combination information obtained in step S623 for each facility ID, procedure ID and type ID of surgical instruments 30. If the number of actual utilization results is less than or equal to the number of combination information in all the determinations in step S624 (step S624: NO), the controller 141 omits processing in step S625 and below, and terminates the processing in FIG. 46.

If the number of actual usage is greater than the number of combination information in one or more determinations in step S624 (step S624: YES), in step S625, controller 141 sends a query to each of the 10 facilities asking whether the number of actual usage is updated to the combination table as in FIG. 39.

When the controller 161 of the work terminal 13 of the facility 10 receives the inquiry sent in step S625 (step S631: YES), the operator of the facility 10 sends a response to the information management system 40 in step S632, indicating whether or not the number of actual usage is acceptable. Specifically, if the operator of facility 10 determines that there is no problem with the received number of actual usage, the operator inputs the answer to the effect that the registration of the number of actual usage to the combination table is permitted to the work terminal 13. On the other hand, if the operator of facility 10 determines that there is a problem with the number of usage results received, the operator inputs a response to the work terminal 13 to the effect that the registration of the number of usage results to the combination table is not permitted. The controller 161 then transmits the entered response to the information management system 40.

When the controller 141 of the information management system 40 receives the response of permission from the work terminal 13 (step S626: YES), it updates the combination information in the combination table similar to FIG. 39 in step S627 with the number of actual usage results obtained in step S622.

On the other hand, if the controller 141 receives a disapproved response from the work terminal 13 (step S626: NO), it omits step S627 and terminates the process in FIG. 46.

In the process of FIG. 46, the controller 141 of the information management system 40 inquired to the facility 10 whether the combination table should be updated with the number of actual usage results, but not limited to this, if the number of actual usage results is greater than the number of combination information, the inquiry may be omitted and the update may be performed automatically. In this case, the controller 141 notifies the corresponding facility 10 of the automatic updating.

### Embodiment 6

In embodiment 6, compared to embodiment 1, a lead time is set for each status of the surgical instruments 30. The lead time is the expected number of days until the surgical instrument 30 becomes available for selection (in stock).

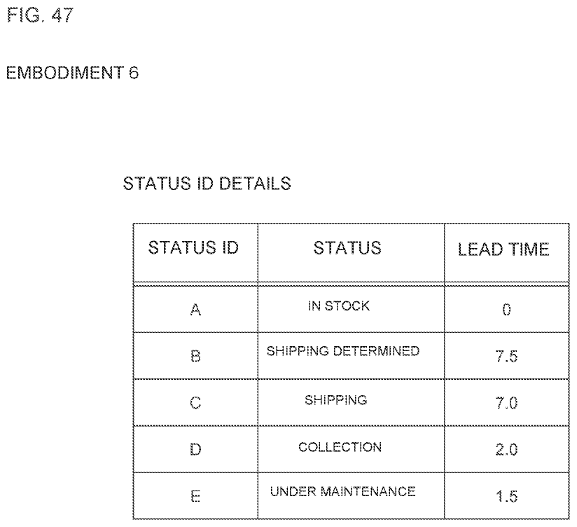

FIG. 47 shows the lead time set for each status ID.

When the value of the status ID is A (in stock), B (shipping determined), C (shipping), D (collection), or E (in maintenance), the expected number of days, or lead time, until the surgical instruments 30 become available for selection (in stock) is 0, 7.5, 7.0, 2.0, and 1.5, respectively. The lead time for each status ID is stored in the database DB in advance.

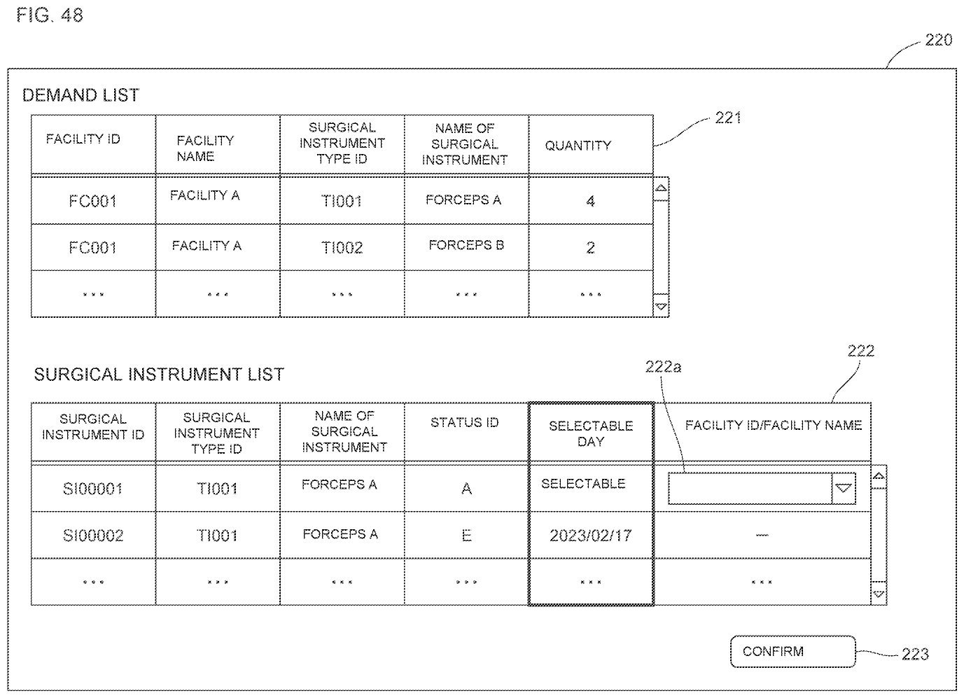

In embodiment 6, in step S412 of FIG. 21, the controller 141 of the information management system 40 transmits surgical instrument information including information on all surgical instruments 30 and a selectable date based on the lead time of the status ID of each surgical instrument 30 to the work terminal 21 of the device center 20. The selectable date is calculated, for example, by adding the lead time for each status ID shown in FIG. 47 to the last update date and time of the surgical instruments 30. This causes the shipping decision screen 220 shown in FIG. 48 to be displayed on the display 173 of the work terminal 21.

FIG. 48 schematically illustrates the shipping decision screen 220 displayed on the display 173 of the work terminal 21 in the device center 20, in accordance with embodiment 6.

In the shipping decision screen 220 of embodiment 6, compared to embodiment 1 shown in FIG. 22, the surgical instrument list 222 includes a selectable date item. When the surgical instrument 30 is selectable (in stock), the selectable date item displays the string “selectable” and the facility ID and facility name items display a pull-down menu 222a. On the other hand, if the surgical instruments 30 are not selectable, the selectable date is displayed in the selectable date item, and “-” is displayed in the facility ID and facility name items, indicating that the facility 10 cannot be selected.

### <Effectiveness of the Method for Managing Surgical Instruments and Information Management System According to Embodiment 6>

As shown in FIG. 47, the status ID (status information) is associated with a lead time (period information) indicating the period of time before the surgical instrument 30 becomes available for selection as a shipping target to the facility 10 through maintenance. As shown in FIG. 48, the lead time (duration information) provides a selectable date (timing information) regarding when the surgical instrument 30 becomes selectable for shipping to the facility 10.

According to this configuration, the quantity of the surgical instruments 30 in stock can be adjusted to take into consideration when the surgical instruments 30 can be selected for shipping to the facility 10, thus preventing the quantity of surgical instruments 30 in stock from being overstocked.

### Embodiment 7

In embodiment 7, compared to embodiment 1, six additional status values are added to the status ID of the surgical instrument 30.

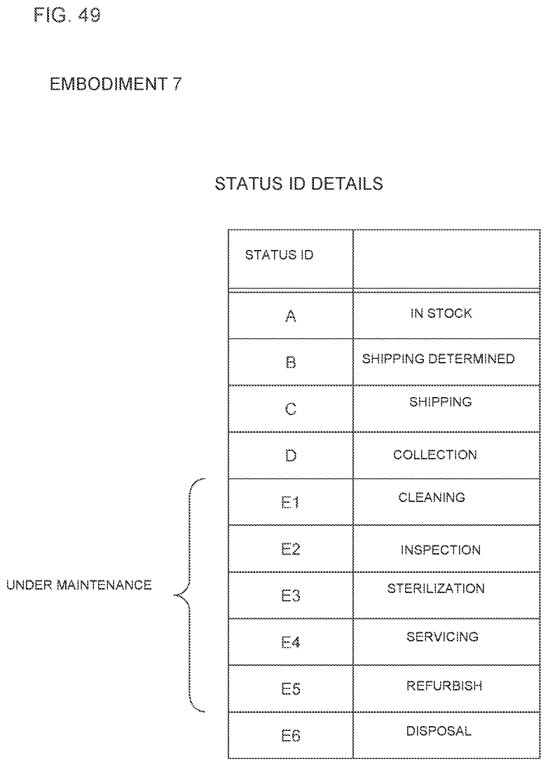

FIG. 49 schematically shows the details of the status ID for embodiment 7.

In embodiment 7, E1 to E6 are added as status ID values compared to embodiment 1 shown in the lower part of FIG. 12. The status indicated by the status ID values E1 to E5 is included in the status indicated by the status ID value E in embodiment 1 (under maintenance).

The status ID value E1 (Cleaning) indicates that the surgical instrument 30 is being cleaned in the maintenance process. The status ID value E2 (Inspection) indicates that the surgical instrument 30 is being inspected in the maintenance process. The inspection includes checking for damage or malfunction of the surgical instruments 30 and checking for exceeding the useful life limit. The value E3 (Sterilization) of the status ID indicates that the surgical instrument 30 is being sterilized in the maintenance process. The status ID value E4 (Repair) indicates that the surgical instrument 30 is being repaired in the maintenance process. Repair indicates that parts are replaced or adjustments are made without disassembling the surgical instrument 30. The status ID value E5 (Refurbish) indicates that the surgical instrument 30 is being refurbished in the maintenance process. Refurbish indicates that the surgical instrument 30 is disassembled and remanufactured by replacing parts, etc. The status ID value E6 (disposed) indicates that the surgical instrument 30 is in a disposed state.

FIG. 50 schematically shows the structure of the surgical instrument table stored in the database DB, according to embodiment 7.

In embodiment 7, compared to embodiment 1 shown in the top row of FIG. 12, the surgical instrument table includes an additional item for cumulative time of use.

In embodiment 7, in step S113 of FIG. 17, the controller 141 of the information management system 40 further calculates the cumulative usage time of the surgical instrument 30 when the surgery using the surgical instrument 30 is completed and the status of the surgical instrument 30 is updated to collection. Specifically, the controller 141 calculates the cumulative usage time by accumulating the time that the surgical instrument 30 has been worn based on the operation log illustrated in FIG. 16. The controller 141 then updates the surgical instrument table with the calculated cumulative usage time.

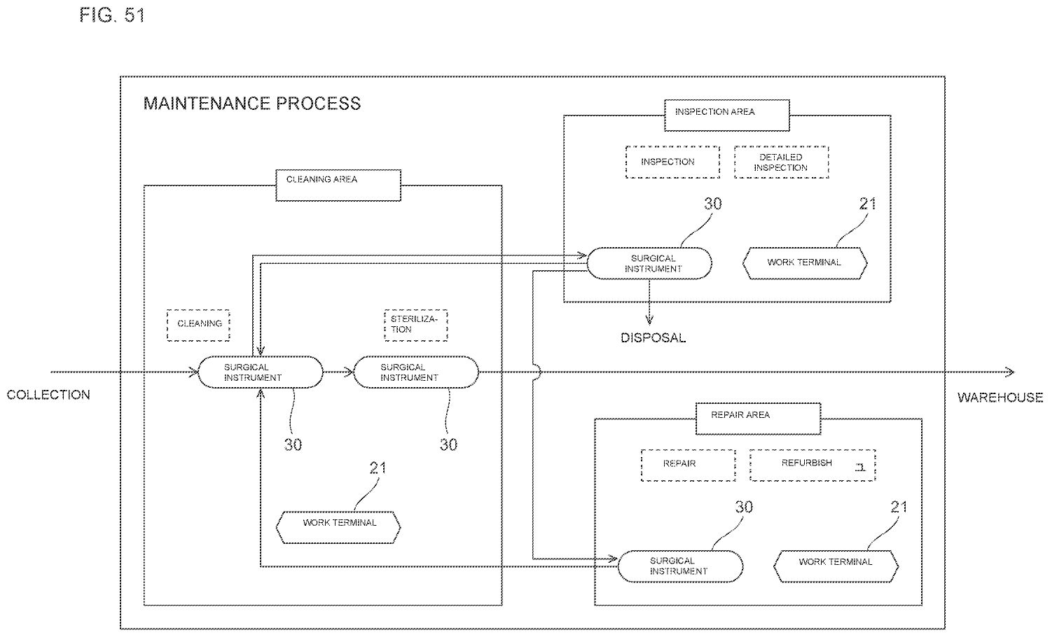

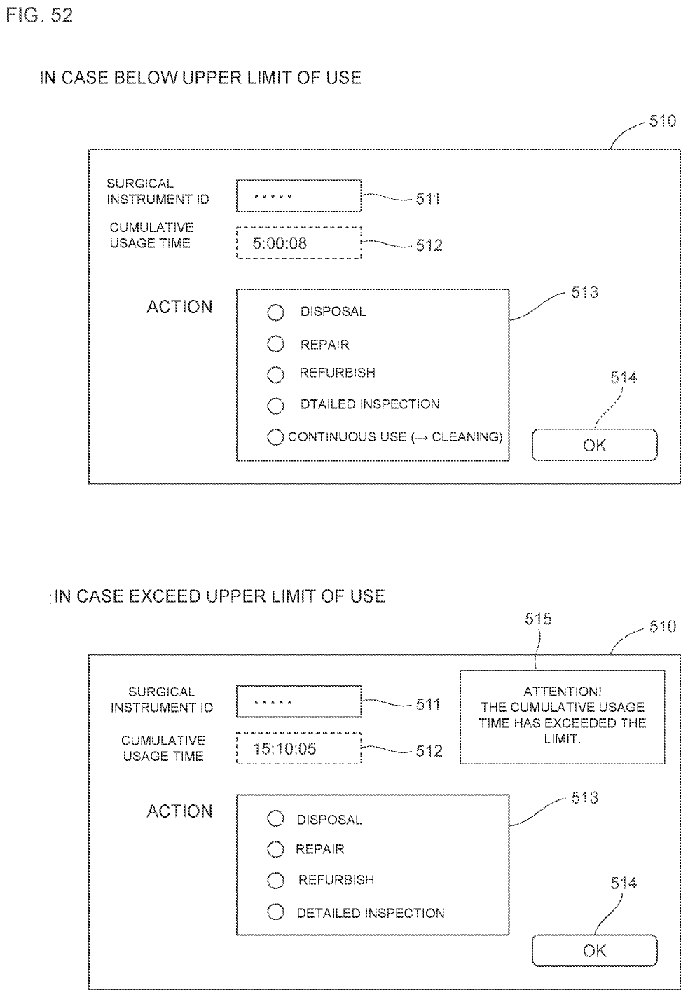

FIG. 51 is a schematic diagram of the maintenance process flow of the device center 20.

In the maintenance process of embodiment 7, cleaning and sterilization are performed in the cleaning area, inspection and detailed inspection are performed in the inspection area, and repair and refurbishing are performed in the repair area.

Once the surgical instruments 30 are collected at the device center 20, the cleaning operator in the cleaning area (hereinafter referred to as “cleaning operator”) updates the status of the collected surgical instruments 30 to cleaning using the work terminal 21. The Cleaning Operator then cleans the recovered surgical instruments 30 according to the process shown in steps S301 to S305 of FIG. 20. After the cleaning is completed, the cleaning operator updates the status of the surgical instruments 30 to inspect using the work terminal 21, and transfers the surgical instruments 30 to the inspection area.

The operator of the inspection area (hereinafter referred to as the “inspection operator”) inspects the surgical instruments 30 and determines whether disposal, repair, refurbish, detailed inspection, or continued use (reuse) should be performed on the surgical instruments 30. If the cumulative usage time of the surgical instruments 30 exceeds the maximum limit, the Inspection Operator will perform a detailed inspection and again make the above decision.

If the inspection operator decides to dispose of the surgical instrument 30, the operator updates the status of the surgical instrument 30 to dispose of it using the work terminal 21, and disposes of the surgical instrument 30. If the inspection operator decides to repair or refurbish the surgical instrument 30, the operator updates the status of the surgical instrument 30 to repair or refurbish using the work terminal 21, respectively, and transfers the surgical instrument 30 to the repair area. If the inspection operator decides to continue using the surgical instrument 30, the operator updates the status of the surgical instrument 30 to clean using the work terminal 21 and transfers the surgical instrument 30 to the cleaning area.

The operator in the repair area (hereinafter referred to as the “repair operator”) repairs surgical instruments 30 whose status is repair and refurbishes surgical instruments 30 whose status is “refurbish”. After the repair or refurbish is completed, the Repair Operator updates the status of the surgical instruments 30 that have been repaired or refurbished to cleaning using the work terminal 21, and transfers the surgical instruments 30 to the cleaning area.

When a surgical instrument 30 returns from the inspection and repair area to the wash area, the wash operator uses work terminal 21 to update the status of the returned surgical instrument 30 to wash and wash the surgical instrument 30. When the cleaning of the surgical instruments 30 returned from the repair area is completed, the cleaning operator updates the status of the surgical instruments 30 to inspect using the work terminal 21, and transfers the surgical instruments 30 to the inspection area again. When the cleaning of the surgical instruments 30 returned from the inspection area is completed, the cleaning operator updates the status of the surgical instruments 30 to sterilization using the work terminal 21. The cleaning operator then sterilizes the surgical instruments 30 according to the process shown in steps S306 and S307 of FIG. 20. When sterilization is complete, the cleaning operator updates the status of the surgical instruments 30 to in stock and transfers the surgical instruments 30 to the warehouse.

FIG. 52 schematically illustrates the configuration of the inspection screen 510 displayed on the display 173 of the work terminal 21 in the inspection area, according to embodiment 7.

The inspection screen 510 has a text box 511, a display area 512, a selection area 513, and an OK button 514. The inspection screen 510 is displayed on the display 173 of the work terminal 21 when an inspection operator inputs instructions to display the inspection screen 510 on the work terminal 21.

The inspection operator uses the reading unit 175 of the work terminal 21 to read the tag 35 of the surgical instrument 30 and enter the surgical instrument ID into text box 211. Alternatively, the operator refers to the string of the surgical instrument ID on the surface of the tag 35 and enters the surgical instrument ID into the text box 511 using the input unit 174 of the work terminal 21. The controller 171 of the work terminal 21 then sends a query including the surgical instrument ID to the information management system 40. Upon receiving the inquiry, the controller 141 of the information management system 40 reads the accumulated usage time corresponding to the surgical instrument ID included in the received inquiry from the surgical instrument table (see FIG. 50) and sends an answer including the read accumulated usage time to the work terminal 21. The controller 171 of the work terminal 21 displays the cumulative usage time included in the received response in the display area 512.

If the cumulative usage time is below a predetermined threshold (upper usage limit), the inspection screen 510 shown in the upper row of FIG. 52 is displayed, and if the cumulative usage time exceeds the predetermined threshold (upper usage limit), the inspection screen 510 shown in the lower row of FIG. 52 is displayed. The upper usage limit for the cumulative usage time is, for example, 10 hours. In the inspection screen 510 shown in the upper part of FIG. 52, the selection area 513 includes radio buttons for determining whether to dispose of, repair, refurbish, conduct a detailed inspection, or continue use. In the lower inspection screen 510 in FIG. 52, the selection area 513 includes radio buttons for determining whether to dispose, repair, refurbish, or perform a detailed inspection, and a message area 515 is added to indicate that the cumulative usage time exceeds the usage limit. The message displayed in message area 515 may be output audibly via a speaker instead of or in addition to the display.

When the inspection operator selects the radio button corresponding to repair, refurbish, or continued use on inspection screen 510 and operates the OK button 514, the status of the subject surgical instrument 30 is updated to repair, refurbish, or clean. Specifically, the controller 171 of the work terminal 21 sends the surgical instrument ID and the status ID corresponding to the radio button to the information management system 40, and the controller 141 of the information management system 40 updates the status ID in the surgical instrument table corresponding to the received surgical instrument ID with the received status ID. The controller 141 of information management system 40 updates the status ID in the surgical instrument table corresponding to the received surgical instrument ID with the received status ID.

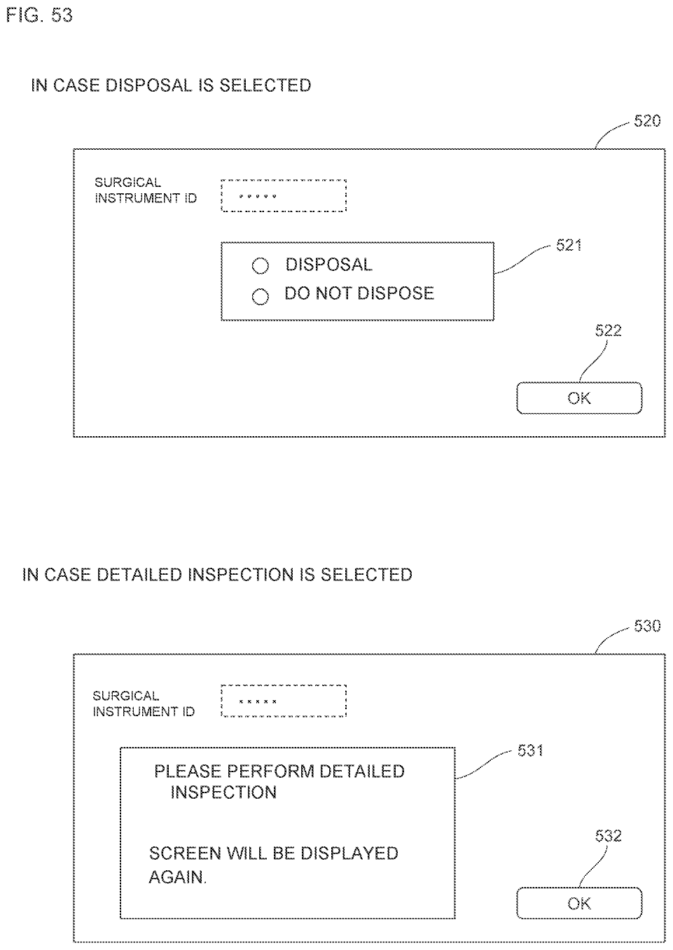

When the inspection operator selects the radio button corresponding to disposal on the inspection screen 510 and operates the OK button 514, a confirmation screen 520 including the selection area 521 and the OK button 522 is displayed, as shown in the upper part of FIG. 53.

When the inspection operator disposes the surgical instrument 30, the inspection operator selects the radio button corresponding to execute the disposal in the selection area 521 and operates the OK button 522. This closes the confirmation screen 530 and updates the status of the subject surgical instrument 30 to discard. The inspection operator then discards the surgical instruments 30. On the other hand, if the inspection operator does not dispose of the surgical instrument 30, he or she selects the radio button corresponding to non-execute disposal in the selection area 521 and operates the OK button 522. This closes the confirmation screen 520, and the upper or lower inspection screen 510 of FIG. 52 is displayed again. Displaying the confirmation screen 520 in this manner prevents the inspection operator from incorrectly setting the disposal.

When the inspection operator selects the radio button corresponding to the detailed inspection on the inspection screen 510 and operates the OK button 514, a confirmation screen 530 including message area 531 and OK button 532 is displayed, as shown in the lower part of FIG. 53. In the message area 531, a message is displayed urging the operator to perform a detailed inspection. The inspection operator confirms this message, inspects the surgical instrument 30 to be inspected in detail, and operates the OK button 532. This closes the confirmation screen 530 and the original inspection screen 510 shown in the upper or lower row of FIG. 52 is displayed again.

Here, if the cumulative usage time exceeds the usage limit, a radio button corresponding to continued use is placed in the selection area 513 on the screen 510 for inspection that is displayed again. Thus, when the cumulative usage time exceeds the upper usage limit, the radio button for continued use is placed only when a detailed inspection is performed. This prevents continuous use from being selected without a detailed inspection when the cumulative usage time exceeds the upper usage limit.

In the confirmation screen 530 shown in the lower part of FIG. 53, a message or button may be placed to confirm whether or not the detailed inspection has passed, and a message or button may be placed to confirm whether or not approval for continued use has been obtained from the responsible person at the device center 20.

### <Effectiveness of the Method for Managing Surgical Instruments and Information Management System According to Embodiment 7>

As shown in FIG. 51, maintenance of the surgical instrument 30 includes inspection of the surgical instrument 30.

According to this configuration, the quality of the surgical instruments 30 can be checked and quality-assured surgical instruments 30 can be provided to the facility 10.

As explained with reference to FIG. 51, inspection of the maintenance process includes inspection whether the surgical instrument 30 can be used (reused) continuously.

According to this configuration, the quality of the surgical instruments 30 to be reused can be assured because the decision to reuse the surgical instruments 30 is made after inspection.

As explained with reference to FIG. 50, when the status of the surgical instrument 30 is updated to collection, the cumulative usage time (usage information regarding the cumulative degree of use) of the surgical instrument 30 is obtained. As explained with reference to FIGS. 52 and 53, maintenance of the surgical instruments 30 includes inspection of the surgical instruments 30 based on the cumulative hours of use (use information).

As the cumulative usage time of the surgical instruments 30 increases, the surgical instruments 30 may deteriorate and become non-reusable. According to the above configuration, since the surgical instruments 30 are inspected based on the accumulated usage time, the accuracy of the inspection of the surgical instruments 30 is enhanced.

As described in FIGS. 52 and 53, the status ID (status information) of the surgical instruments 30 designated for disposal based on the disposal condition is updated to indicate that they cannot be selected for shipping to the facility 10.

Disposal conditions include, for example, that the cumulative degree of use has exceeded the upper limit of use, or that a detailed inspection by the inspection operator has determined that continued use is not possible. For example, if a defect such as a scratch is found on the surgical instrument 30 in a post-cleaning inspection, the above configuration can prevent the surgical instrument 30 from being reused by selecting it for disposal.

### Modification 1 of Embodiment 7

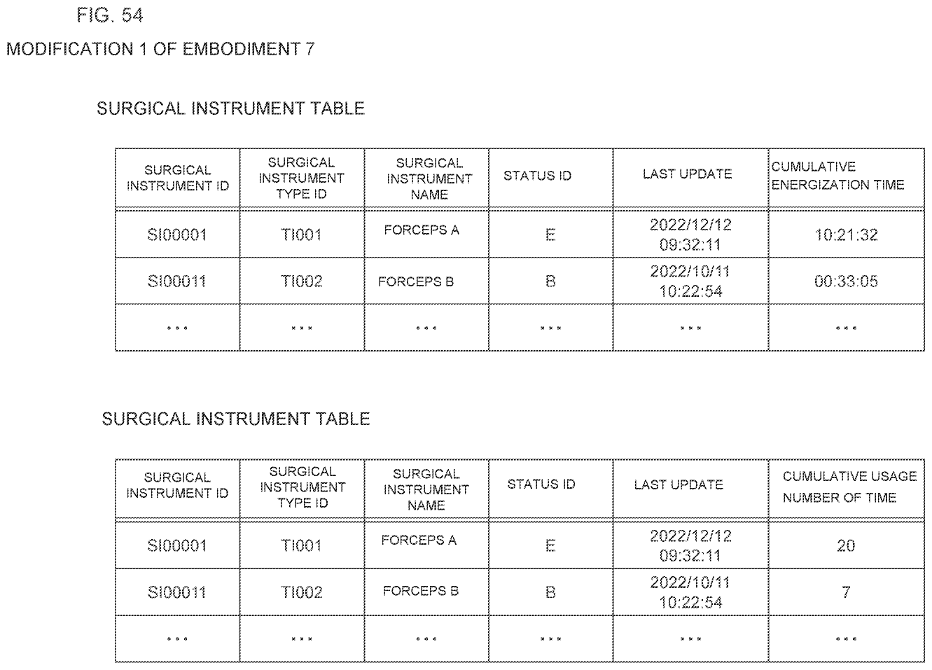

In embodiment 7, the cumulative usage time was the total time the surgical instrument 30 was worn, but it can also be the cumulative energization time, which is the accumulated time that an operation was performed on the surgical instrument 30 (e.g., energization time), as shown in the top row of FIG. 54. The cumulative energization time can be calculated based on the operation flags for each type of operation described above. In this case, the operation log can include the energized state of the surgical instrument 30 as well as the attachment and removal of the surgical instrument 30.

In embodiment 7, the cumulative usage time was used as usage information regarding the cumulative degree of use, but it may not be limited to this, and the cumulative number of usage may also be used. The cumulative number of usage may be counted up according to the fact that the surgical instrument 30 has been used in one surgery, or according to the fact that the surgical instrument 30 has been attached to the surgical robot system 11.

In both the upper and lower sections of FIG. 54, the accuracy of the inspection of the surgical instrument 30 is enhanced because the inspection of the surgical instrument 30 is based on the cumulative energization time or the cumulative number of usage (use information regarding the cumulative degree of use). The cumulative degree of usage may be calculated based on two or more of the cumulative wearing time, cumulative energization time, and cumulative number of usage.

### Modification 2 of Embodiment 7

In the inspection of the maintenance process of embodiment 7, the operation of the surgical instruments 30 may be inspected using the surgical robot system 11.

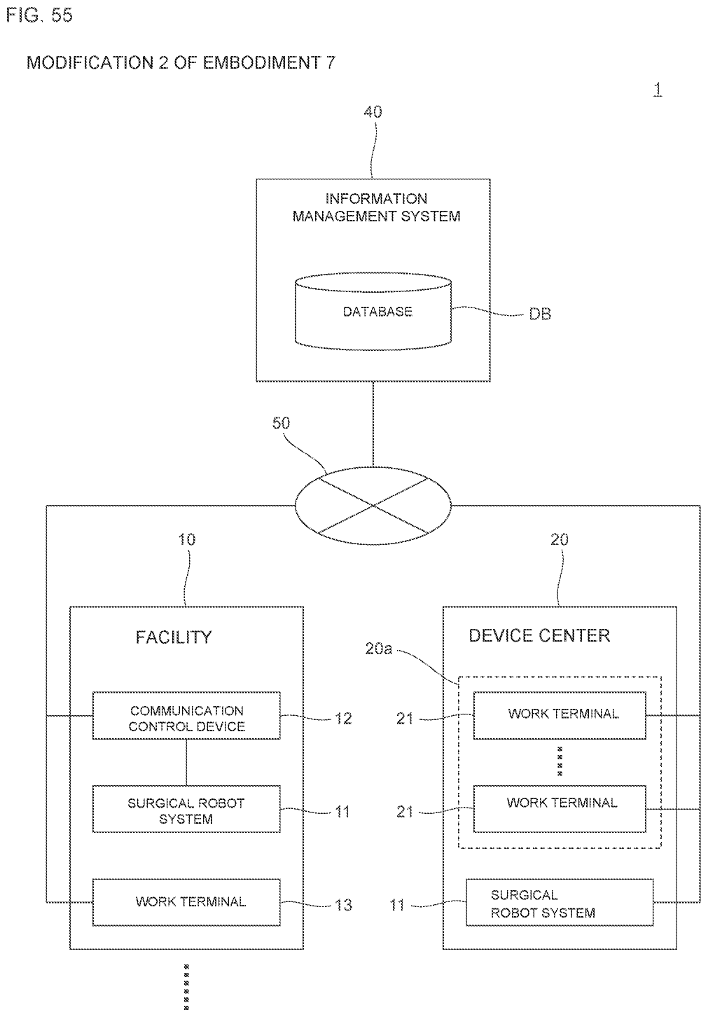

FIG. 55 is a block diagram showing the functional structure of the surgical instrument management system 1 for this modification.

The device center 20 of embodiment 7 is further equipped with a surgical robot system 11 as compared to embodiment 1 shown in FIG. 2. The surgical robot system 11 in the device center 20 is located in the inspection area (see FIG. 51) and is connected to the information management system 40 via the network 50. An inspection operator attaches a surgical instrument 30 to the surgical robot system 11 to be inspected and operates the surgical instrument 30 using the surgical robot system 11 to inspect the surgical instrument 30.

Here, the operation apparatus 120 transmits drive instructions to the control device 130 in response to the operation. The control device 130 generates command values based on the drive instructions received from the operation apparatus 120 and transmits the generated command values to the patient-side device 110. The patient-side device 110 operates the surgical instruments 30 together with the parts of the patient-side device 110 according to the command values transmitted from the control device 130. On the other hand, the patient-side device 110 transmits the drive amount output from the encoder of each operating section to the control device 130 as the current value. The control device 130 transmits the command value and the current value corresponding to the command value to the information management system 40 at predetermined time intervals (e.g., 1 second).

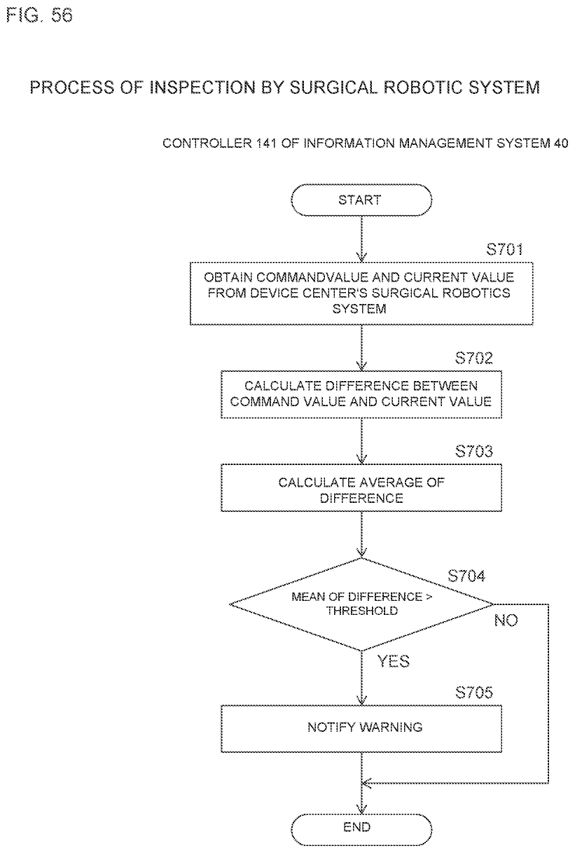

FIG. 56 is a flowchart showing the process of inspection using the surgical robot system 11 for this modification.

In the inspection area of the device center 20, the inspection operator attaches the surgical instrument 30 to the patient-side device 110 of the surgical robot system 11 for inspection and operates the patient-side device 110 to operate the surgical instrument 30 against a standard object for inspection, such as a simulated organ. As a result, command values and current values are transmitted from the control device 130 of the surgical robot system 11 in the device center 20 to the information management system 40.

In step S701, the controller 141 of the information management system 40 obtains command values and current values from the surgical robot system 11 of the device center 20 and stores the obtained command values and current values in the database DB. After the operation of the surgical robot system 11 in the inspection is completed, the controller 141 calculates the difference between the command value and the current value corresponding to the operation of the surgical instrument 30 for each pair in step S702, and calculates the average value of each difference in step S703. In step S704, the controller 141 determines whether the average value of the differences calculated in step S703 is greater than a predetermined threshold value. For example, if the surgical instrument 30 operated normally, the average value of the differences is smaller, and if the surgical instrument 30 did not operate normally, the average value of the differences is larger. Therefore, by comparing the average value of the differences with the predetermined threshold value, it is possible to determine whether the surgical instrument 30 is normal or not.

If the average value of the differences is greater than the predetermined threshold value (step S704: YES), in step S705, the controller 141 sends a notification to the surgical robot system 11 of the device center 20 to display a warning on the display of the operation unit 112 (see FIG. 6). When the warning is displayed on the display of the operation unit 112, the inspection operator can grasp that the surgical instrument 30 to be inspected attached to the surgical robot system 11 is not working properly.

The patient-side device 110 may transmit the current value applied to the surgical instrument 30 when operating the surgical instrument 30 to the information management system 40, including the current value. In this case, the controller 141 of the information management system 40 calculates the difference between the command value converted to correspond to the current value and the current value for each pair. For example, if the surgical instrument 30 is a grasping forceps, the wires in the shaft 32 of the forceps may have been stretched and loosened by previous use. In this case, when the operation is performed on a standard object for inspection, such as a simulated organ, a larger current value than usual is required, resulting in a larger difference between the command value and the current value. Therefore, in this case also, if a warning notification is sent when the average value of the difference is greater than a predetermined threshold, the inspection operator can know that the grasping forceps for the sample object is not operating normally.

### Embodiment 8

In embodiment 8, a usage status ID is further added to the status ID of embodiment 7 (see FIG. 49).

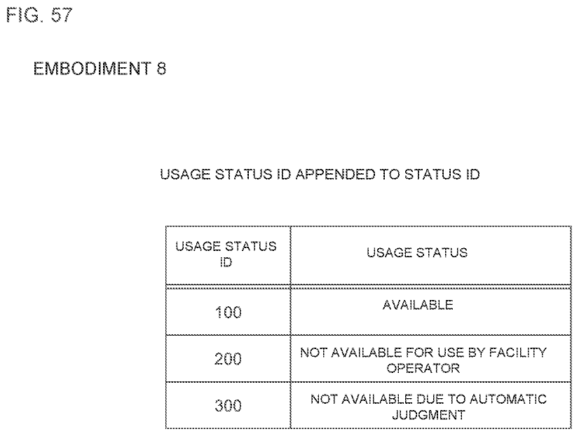

FIG. 57 shows the usage status ID added to the status ID for embodiment 8.

The use state ID contains three different values: 100, 200, and 300. A value of 100 for the use state ID indicates that the surgical instrument 30 is ready for use. The value 200 of the use state ID indicates that the surgical instrument 30 is set to disabled for use by the operator of the facility 10. A value of 300 for the use information ID indicates that the device has been set to disabled for use by automatic determination. Disable by automatic determination is set when the use information regarding the cumulative degree of use (e.g., cumulative wearing time, cumulative energizing time, and cumulative number of usage) exceeds a predetermined threshold value.

For example, if the surgical instrument 30 is kept in inventory and available for use, the status ID of the surgical instrument 30 is “A-100”, which is the combination of A indicating the inventory status and 100 indicating the available status. In this case, the surgical instrument 30 with status ID A-100 is displayed in the surgical instrument list 222 shown in FIG. 22.

If the operator of facility 10 determines that there was a problem with the surgical instruments 30 used in the surgery, the operator of facility 10 will update the status ID of the surgical instruments 30 to “D-200” (collection and unavailable for use by the operator of facility 10) using work terminal 13.

The work terminal 13 may display a screen for accepting additional information regarding problems with the surgical instruments 30, and may accept additional information from the operator regarding problems with the surgical instruments 30. In such a case, the accepted additional information may be transmitted from the work terminal 13 to the information management system 40, and the information management system 40 may store the received additional information in association with the surgical instrument ID of the corresponding surgical instrument 30. The additional information may be text, images, and audio, or a combination thereof.

If a surgical instrument 30 that has been set as unusable by the operator of facility 10 is transferred to the inspection area, the status ID of this surgical instrument 30 is updated to “E2-200” (status of inspection and status of unusable by the operator of facility 10). In this case, the inspection operator decides to repair or refurbish the surgical instrument 30. If the results of the inspection after repair or refurbish indicate that the surgical instrument 30 is ready for continued use, the status ID of the surgical instrument 30 is updated to “E1-100” (cleaned and ready for use) and the instrument is transferred to the cleaning area.

In embodiment 8, when displaying the inspection screen 510, etc. shown in FIG. 52, the controller 141 of the information management system 40 sets the status ID of the surgical instrument 30 to “E2-300” (status of inspection and status of disuse by automatic judgment) when the controller 30 determines that the usage information (e.g., cumulative wearing time, cumulative energization time, and cumulative number of usage) regarding the cumulative degree of use for the surgical instrument 30 to be inspected has exceeded a predetermined threshold value. This prevents the surgical instruments 30 with a use status ID value of 300 from being mistakenly selected for shipping.

### Modification of Embodiment 8

In embodiment 8, a usage status ID is added to the status ID of embodiment 7 (see FIG. 49), but a refurbish ID indicating the refurbish count may be added in place of the usage status ID, as shown below.

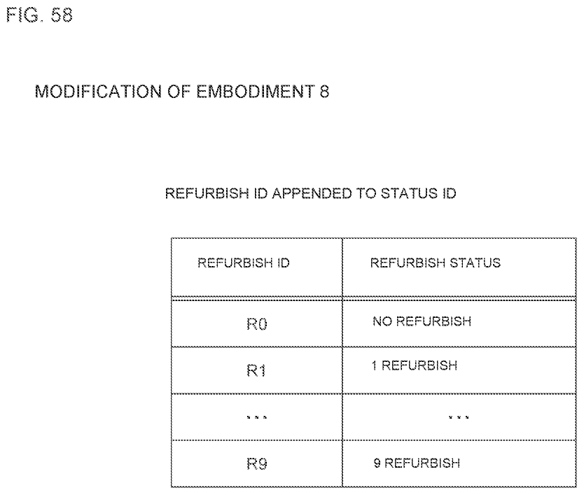

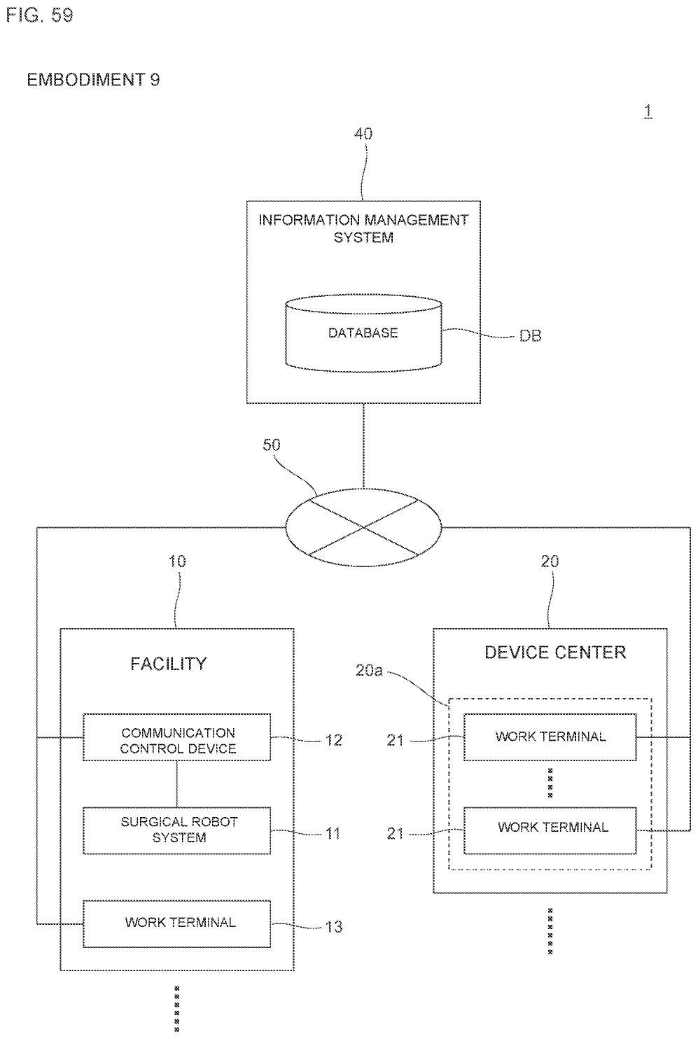

FIG. 58 shows the refurbish ID added to the status ID for this modification.

The refurbish ID contains 10 different values, R0 to R9. The subscripts (numeric part) of the values R0 to R9 indicate the number of times the refurbish has been performed. The value R0 of the refurbish ID indicates that no refurbish has ever been performed on the surgical instrument 30. Refurbish R1 to R9 indicates that refurbishing has been performed 1 to 9 times for the surgical instrument 30, respectively. Each time the controller 141 of the information management system 40 updates the status of the surgical instrument 30 to refurbish in the repair area, the subscript of the refurbish ID assigned to the status ID of the surgical instrument 30 is increased by one.

When displaying the inspection screen 510, etc. shown in FIG. 52, the controller 141 of the information management system 40 obtains the refurbish count of the surgical instruments 30 by obtaining the subscript of the refurbish ID added to the status ID. The controller 141 transmits the acquired refurbish count to the work terminal 21 in the inspection area. As a result, the refurbish count of the surgical instruments 30 to be inspected is displayed on the inspection screen 510 shown in FIG. 52. Thus, the inspection operator can determine the treatment for the surgical instruments 30 to be inspected with reference to the refurbish count.

When the use status ID of embodiment 7 is used together with the refurbish ID, the controller 141 of the information management system 40 may, when the refurbish count exceeds a predetermined threshold (e.g., 9 times), add to the status ID of the surgical instrument 30 in question a use status ID value indicating that the instrument cannot be used by automatic determination 300 may be added. A repair ID indicating the number of times the device has been repaired may also be added to the status ID.

### Embodiment 9

In embodiment 9, compared to embodiment 1, the surgical instrument management system 1 has multiple device centers 20, as shown in FIG. 59.

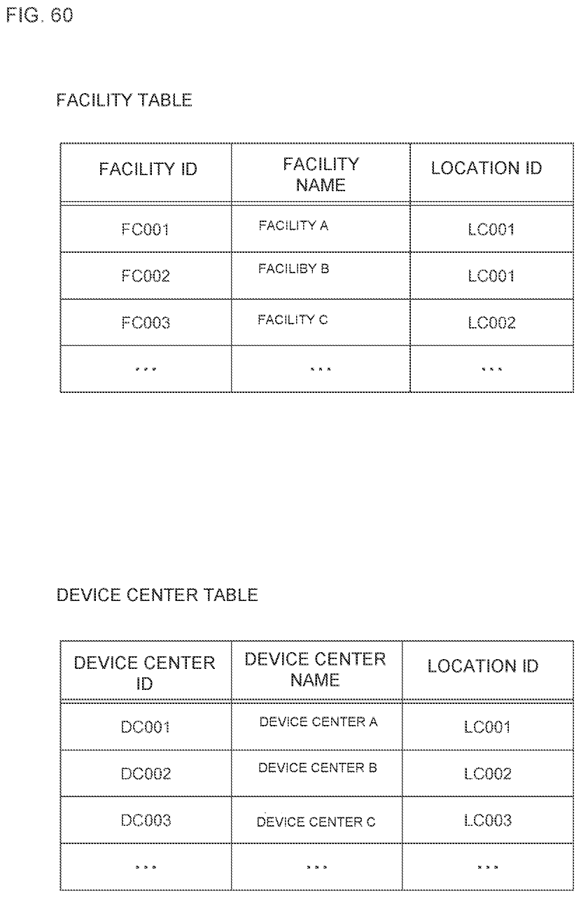

FIG. 60 schematically shows the structure of the facility table and device center table stored in the database DB, according to embodiment 9.

As shown in the top row of FIG. 60, the facility table includes entries for facility ID, facility name, and location ID. As shown in the bottom row of FIG. 60, the device center table includes entries for device center ID, device center name, and location ID. The device center ID is an ID that can identify device centers 20 individually. The device center name indicates the name of the device center 20 indicated by the device center ID.

The location ID is an ID that can individually identify the region in which the device center 20 is located. In the example shown in FIG. 60, one device center ID corresponds to one location ID. If multiple facilities 10 exist in the region corresponding to one location ID, the location IDs of these multiple facilities 10 will be common. Multiple location IDs correspond, for example, to two regions in eastern and western Japan, or three regions in northern, eastern, and western Japan, in the case of Japan.

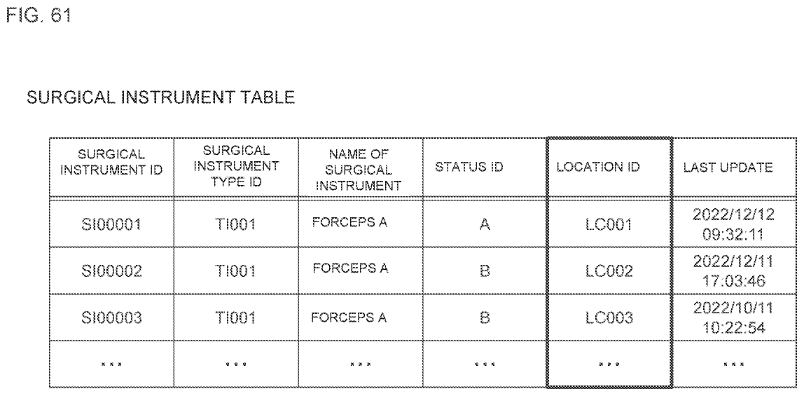

FIG. 61 schematically shows the structure of the surgical instrument table stored in the database DB, according to embodiment 9.

The surgical instrument table of embodiment 9 includes an additional location ID entry compared to embodiment 1 shown in the top row of FIG. 12.

In embodiment 9, the same process as in FIG. 21 is performed in the selection process. However, in embodiment 9, the location ID of the device center 20 of the transmission source is added to the request instruction transmitted in step S402 of FIG. 21. In step S412 of FIG. 21, the controller 141 of the information management system 40 obtains the facility ID corresponding to the location ID based on the location ID of the device center 20 of the transmission source and the facility table shown in the upper part of FIG. 60, and obtains the demand information corresponding to the obtained facility ID from the demand information from the same demand information table shown in FIG. 13. The controller 141 also obtains the surgical instrument information for the surgical instruments 30 that correspond to this location ID and whose status ID value is A (in stock) based on the location ID of the device center 20 of the transmission source and the surgical instrument table shown in FIG. 61.

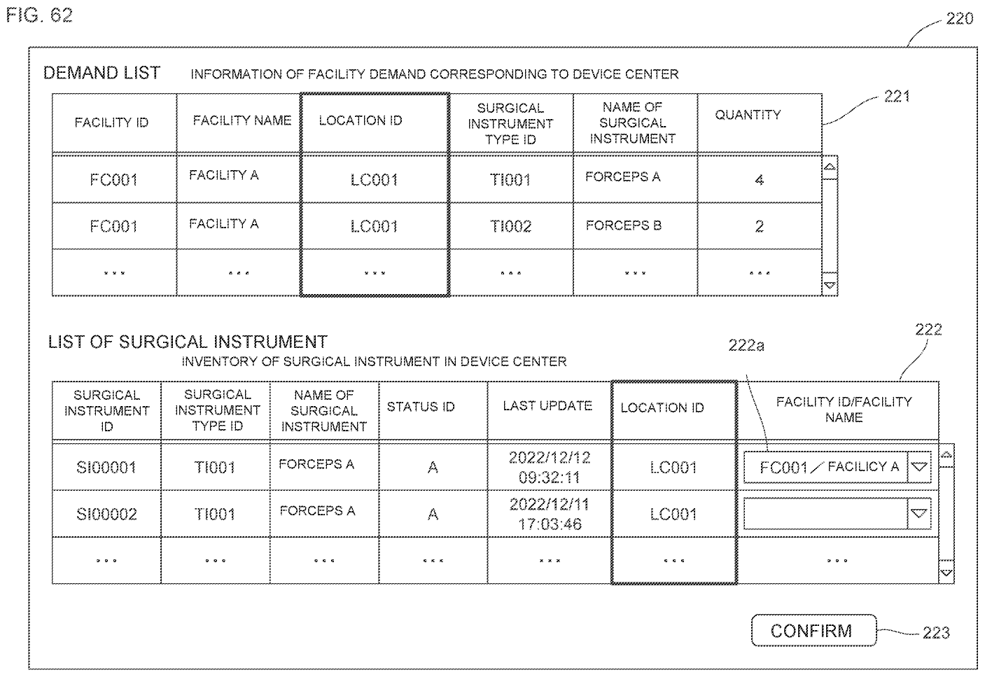

FIG. 62 schematically illustrates the shipping decision screen 220 displayed on the display 173 of the work terminal 21 in the device center 20, in accordance with embodiment 9.

In the shipping decision screen 220 of embodiment 9, compared to embodiment 1 shown in FIG. 22, both the demand list 221 and the surgical instrument list 222 include a location ID item. The demand list 221 displays demand information for the facility 10 located at the same location as the device center 20 where the shipping decision screen 220 is displayed. The surgical instrument list 222 displays the inventory of surgical instruments 30 at the device center 20 where the shipping decision screen 220 is displayed. In this case, the surgical instruments 30 to be delivered to facility 10 are delivered from the warehouse of device center 20 in the same location as that facility 10.

The surgical instruments 30 delivered to a facility 10 may be delivered from the inventory of device center 20 in the same location as that facility 10, as well as from the inventory of device center 20 in a different location than that facility 10. In this case, the demand list 221 displays the demand information for all facilities 10, and the surgical instrument list 222 displays the inventory of surgical instruments 30 at all device centers 20. The surgical instruments 30 to be delivered to the 10 facilities should be delivered from the device center 20 in the same location as much as possible to reduce shipping time. If there is an imbalance in the inventory counts of two device centers 20 in different locations, the surgical instruments 30 in the warehouses of the two device centers 20 may be transferred between each other's warehouses so that the imbalance is eliminated.

### <Effectiveness of the Method for Managing Surgical Instruments and Information Management System According to Embodiment 9>

As shown in FIG. 61, for each of the multiple surgical instruments 30, a location ID (location information about the current location of the surgical instruments 30) is stored in association with the surgical instrument ID (identification information) for each of the multiple surgical instruments 30.

According to this configuration, for example, surgical instruments 30 located close to facility 10 can be delivered to the facility 10, thus enabling efficient shipping of surgical instruments 30 to facility 10.

### Embodiment 10

In embodiment 10, compared to embodiment 1, in step S412 of FIG. 21, the controller 141 of the information management system 40 calculates the elapsed time since the maintenance was completed, in other words, the elapsed time since the status became in stock, for the surgical instruments 30 whose status is in stock. Specifically, the controller 141 calculates the elapsed time from the last update date and time to the current time for the surgical instrument 30 whose status ID value is A (in stock). The controller 141 then includes the elapsed time for each surgical instrument 30 in the surgical instrument information.

The controller 141 of the information management system 40 may obtain the elapsed time since the completion of maintenance by referring to the elapsed time entry in the surgical instrument table, not limited to calculating it each time upon request when displaying the shipping decision screen 220 as described above. In this case, the controller 141 updates the elapsed time in the surgical instrument table every hour, for example.

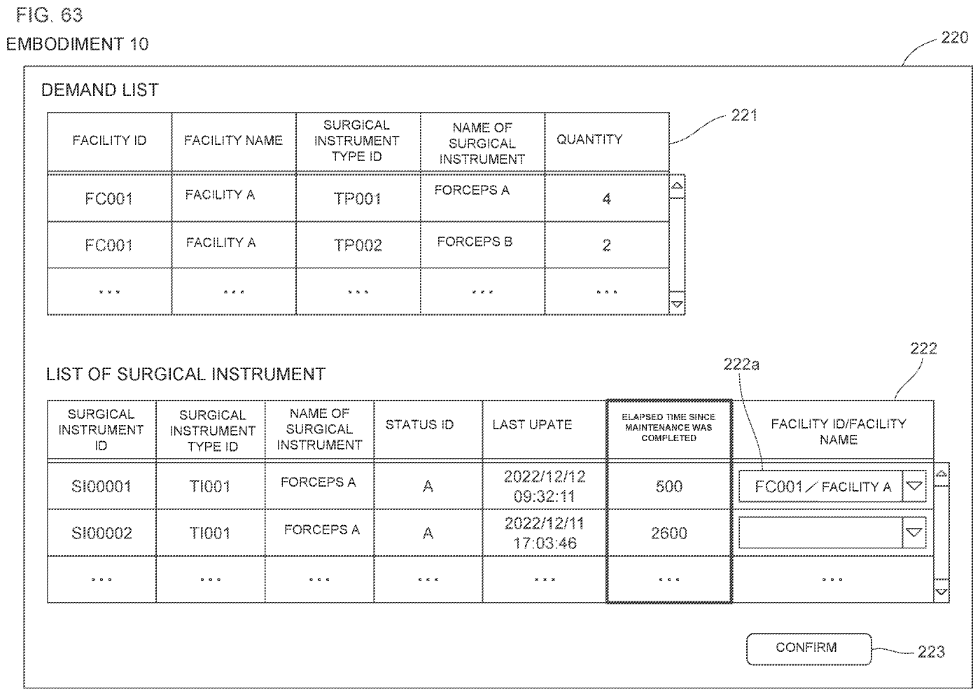

FIG. 63 schematically illustrates the shipping decision screen 220 displayed on the display 173 of the work terminal 21 in the device center 20, according to embodiment 10.

In the shipping decision screen 220 of embodiment 10, compared to embodiment 1 shown in FIG. 22, the surgical instrument list 222 includes an item for the elapsed time since maintenance was completed. In this case, the operator in the selection process determines the items in the surgical instrument list 222 as shipping targets in order of elapsed time since maintenance completion. This prevents the surgical instruments 30 from being stored in the warehouse for a long period of time.

The controller 141 of the information management system 40 calculates the elapsed time since the completion of maintenance for the surgical instruments 30 in inventory status at each predetermined timing (e.g., one hour), and if the calculated elapsed time exceeds a predetermined threshold (e.g., 2500 hours), the status of the surgical instruments 30 in question The status of such surgical instrument 30 may be changed to “under maintenance”. Such surgical instruments 30 are again cleaned and sterilized.

The controller 141 of the information management system 40 may calculate the date and time of expiration by adding a predetermined threshold value (e.g., 2500 hours) to the update date and time when the status of the surgical instrument 30 is updated to in stock.

In this case, the controller 141 transmits the calculated date and time of expiration date and the surgical instrument ID corresponding to the expiration date to the work terminal 21 of the device center 20. The operator of the device center 20 uses the work terminal 21 to store the received date and time of expiration in the memory 31 (see FIG. 5) of the corresponding surgical instrument 30. When the surgical instrument 30 is attached to the patient-side device 110 of the surgical robot system 11 of the facility 10, the controller 131 of the surgical robot system 11 reads the expiration date and time from the memory 31. If the current date/time is past the read-out expiration date, the controller 131 displays a warning on the display of the operation unit 112 indicating that the expiration date of the surgical instrument 30 has passed. This prevents the use of the surgical instrument 30 whose expiration date has passed.

### Embodiment 11

In embodiment 11, compared to embodiment 1, a deliverable flag entry is added to the surgical instrument table.

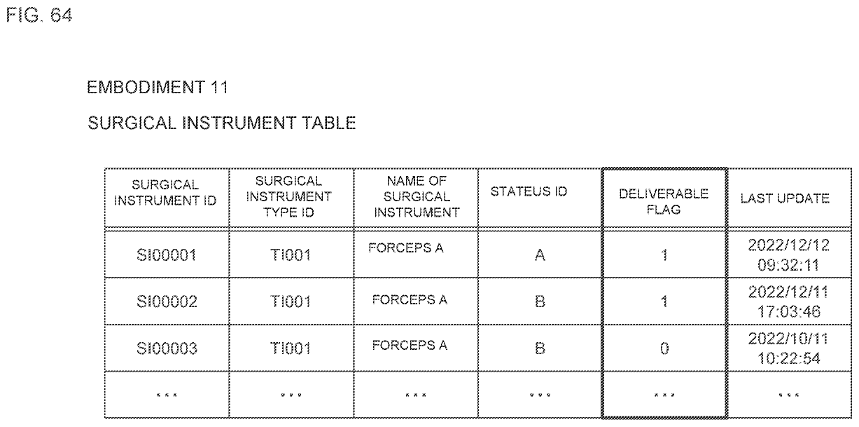

FIG. 64 is a block diagram schematically showing the structure of the surgical instrument table stored in the database DB, according to embodiment 11.

The deliverability flag is status information indicating whether or not the corresponding surgical instruments 30 are deliverable, i.e., whether or not they are in the status of “A: In Stock” in embodiment 1, where “0” indicates that shipping is not possible and “1” indicates that shipping is possible. The initial value of the deliverable flag is “0”. In embodiment 11, the controller 141 of the information management system 40 updates the deliverable flag from “0” to “1” in step S212 (see FIG. 18), along with updating the status ID.

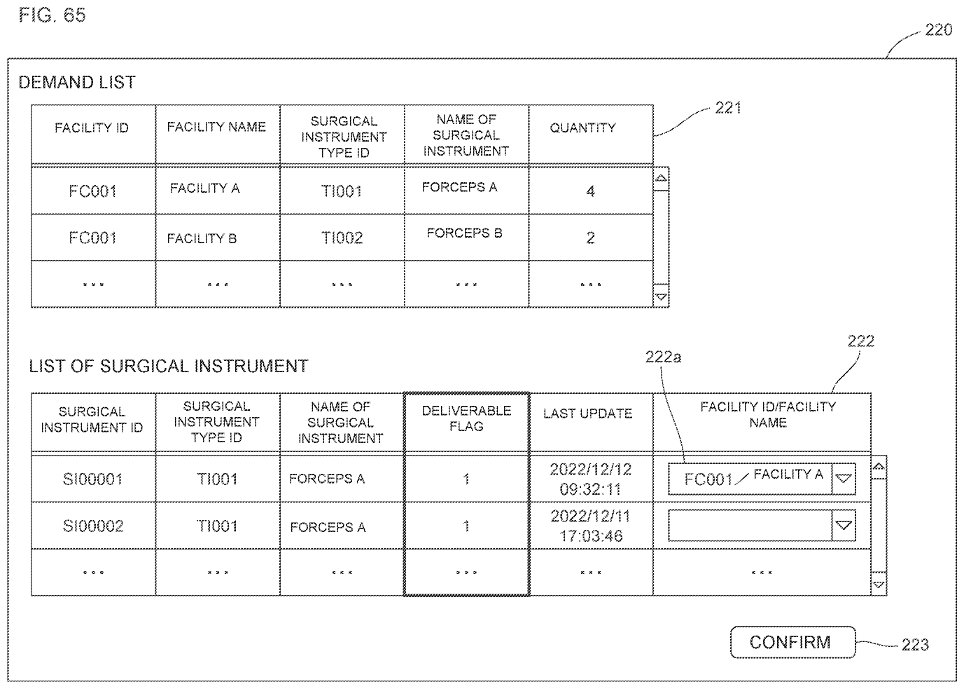

FIG. 65 schematically illustrates the shipping decision screen 220 displayed on the display 173 of the work terminal 21 in the device center 20, according to embodiment 11.

The list of surgical instruments 30 with the deliverable flag set to “1” is displayed in the surgical instrument list 222 of the shipping decision screen 220, and the deliverable flag is displayed instead of the status ID. The display of the deliverable flag may be omitted in the surgical instrument list 222.

### Example of Modification of Embodiment 11

The status ID entry may be deleted from the surgical instrument table and the status ID update process in the information management system 40 may be omitted.

### <Effectiveness of the Method of Managing Surgical Instruments and Information Management System According to Embodiment 11 and its Variants>

As in embodiment 1, maintenance such as cleaning and sterilization of surgical instruments 30 used in the surgical robot system 11 does not need to be performed at the facility 10 where the surgical robot system 11 is installed, thus reducing the burden on the facility 10 that uses the surgical robot system 11. In addition, since it is possible to manage whether or not the surgical instruments 30 can be selected for shipping to facility 10 based on the deliverability flag of the surgical instruments 30, the surgical instruments 30 that become deliverable through maintenance can be identified from among multiple surgical instruments 30 and provided to facility 10 for reuse.

### Other Examples of Changes

In embodiments 1 to 11, the status ID and the last update date/time are stored in the surgical instrument table of the database DB, but the status ID and the last update date/time may be stored in the memory 31 of the surgical instrument 30 (see FIG. 5). In this case, an interface for connecting the surgical instruments 30 is provided in the work terminals 13 and 21, and the work terminals 13 and 21 read the status ID and the last update date and time from the memory 31 of the surgical instruments 30 via the interface, and also the status ID and the last update date and time are updated.

In embodiments 1 to 11, the status of the surgical instruments 30 delivered to facility 10 was updated to collection when the surgery was completed, but the status may be updated to, for example, “Facility” upon shipping to facility 10. In this case, the operator in charge of receiving at facility 10 reads the surgical instrument ID from the surgical instrument 30 delivered to facility 10 using the reading unit 165 of work terminal 13. The work terminal 13 then transmits the surgical instrument ID read by the work terminal 13 to the information management system 40, thereby updating the status of the surgical instrument 30 to “facility”.

As described above, the method for managing surgical instruments and the information management system for one or more embodiments may allow reuse of surgical instruments used in a surgical robotic system while reducing the burden of maintenance of surgical instruments at facilities using the surgical robotic system.

One or more embodiments may be modified in various ways as appropriate within the scope of the technical concept.

As a supplement, summarize one or more embodiments of a surgical instrument management method and information management system.

A method of managing surgical instruments, according to one or more embodiments, that are used in surgical robotic systems installed in each of facilities, that are collected, for each of which a maintenance comprising at least cleaning and sterilization is performed, and that are shipped to any one of the facilities, which may include:
storing, for each of the surgical instruments, status information regarding status of the surgical instruments in association with identification information for each of the surgical instruments, and
updating the status information corresponding to a surgical instrument that has been made available for shipping by the maintenance to the status information indicating that the surgical instrument is available to be selected for shipping to any one of the facilities.

In the method of managing surgical instruments according to one or more embodiments, the surgical instrument made available for shipping by the maintenance comprises a surgical instrument that maintenance is completed and in stock status.

In the method of managing surgical instruments according to one or more embodiments, the method of managing surgical instruments may further include updating status information corresponding to a surgical instrument that has been selected for shipping to any one of the facilities to the status information indicating that the surgical instrument is not available to be selected for shipping to the facilities.

In the method of managing surgical instruments according to one or more embodiments, the surgical instruments may be shared by the surgical robotic systems installed in each of the facilities.

In the method of managing surgical instruments according to one or more embodiments, the method of managing surgical instruments, may further include storing demand information regarding types and quantities of surgical instruments that need to be shipped to any one of the facilities; and providing the demand information for the selection of surgical instruments to be shipped to any one of the facilities.

In the method of managing surgical instruments, the method of managing surgical instruments may further include: generating surgical instrument information indicating surgical instruments that are available to be selected for shipping to any one of the facilities based on the status information; and providing the surgical instrument information along with the demand information.

In the method of managing surgical instruments according to one or more embodiments, the demand information may include surgery schedule information regarding scheduled surgeries using the surgical robot systems at the facilities.

In the method of managing surgical instruments according to one or more embodiments, the surgery schedule information may include information regarding the scheduled surgery date on which a surgery using one of the surgical robot systems is performed.

In the method of managing surgical instruments according to one or more embodiments, the providing the demand information may include providing the demand information at a time according to the scheduled surgery date.

In the method of managing surgical instruments according to one or more embodiments, the surgery schedule information may include at least one of content information regarding a content of a surgery, and surgeon information regarding a surgeon operating one of the surgical robot systems.

In the method of managing surgical instruments according to one or more embodiments, the providing the demand information may include generating the demand information based on at least one of the content information and the surgeon information, and information regarding a combination of the surgical instruments that need to be shipped to one of the facilities.

In the method of managing surgical instruments according to one or more embodiments, the status information is associated with time period information indicating a period of time until each of the surgical instruments becomes available to be selected for shipping to any one of the facilities by the maintenance, the method further comprises providing, based on the time period information, timing information indicating when one of the surgical instruments will be available to be selected for shipping to one of the facilities.

In a method of managing surgical instruments according to one or more embodiments, the maintenance may include inspection of each of the surgical instruments.

In the method of managing surgical instruments according to one or more embodiments, the inspection may include an inspection that determines if each of the surgical instruments is reusable.

In the method of managing surgical instruments according to one or more embodiments, the method may further comprise obtaining usage information regarding the cumulative degree of use of each of the surgical instruments, wherein the maintenance may include inspection of each of the surgical instruments based on the usage information.

In the method of managing surgical instruments according to one or more embodiments, the usage information may relate to at least one of cumulative number of times of use of each of the surgical instruments and a cumulative duration of use each of the surgical instruments.

In the method of managing surgical instruments according to one or more embodiments, the method may further include updating the status information for a surgical instrument designated for disposal based on disposal condition to the status information indicating that the surgical instrument is not selectable for shipping to the facilities.

In the method of managing surgical instruments according to one or more embodiments, the demand information may be generated based on utilization information regarding an actual usage of the surgical instruments at one of the facilities.

In the method of managing surgical instruments according to one or more embodiments, the method may further include outputting types and quantities of surgical instruments that are selectable for shipping to any one of the facilities based on the status information.

In the method of managing surgical instruments according to one or more embodiments, the method may further include storing, for each of the surgical instruments, location information regarding the current location of each of the surgical instruments in association with the identification information of each of the surgical instruments.

An information management system that manages surgical instruments that are used in surgical robotic systems installed in each of facilities, that are collected, for each of which a maintenance comprising at least cleaning and sterilization is performed, and that are shipped to any one of the facilities, comprising:
a memory that stores, for each of the surgical instruments, status information regarding status of the surgical instruments in association with identification information for each of the surgical instruments; and
a controller that updates the status information corresponding to a surgical instrument that has been made available for shipping by the maintenance to the status information indicating that surgical instrument is available to be selected for shipping to any one of the facilities.

1. A method of managing surgical instruments that are used in surgical robotic systems installed in each of facilities, that are collected, for each of which a maintenance comprising at least cleaning and sterilization is performed, and that are shipped to any one of the facilities, comprising:
storing, for each of the surgical instruments, status information regarding status of the surgical instruments in association with identification information for each of the surgical instruments, and
updating the status information corresponding to a surgical instrument that has been made available for shipping by the maintenance to the status information indicating that the surgical instrument is available to be selected for shipping to any one of the facilities.

2. The method of managing surgical instruments according to claim 1, wherein
the surgical instrument made available for shipping by the maintenance comprises a surgical instrument that the maintenance is completed and in stock status.

3. The method of managing surgical instruments according to claim 1, further comprising
updating the status information corresponding to a surgical instrument that has been selected for shipping to any one of the facilities to the status information indicating that the surgical instrument is not available to be selected for shipping to the facilities.

4. The method of managing surgical instruments according to claim 1, wherein
the surgical instruments are shared by the surgical robotic systems installed in each of the facilities.

5. The method of managing surgical instrument according to claim 1, further comprising:
storing demand information regarding types and quantities of surgical instruments that need to be shipped to any one of the facilities; and
providing the demand information for the selection of surgical instruments to be shipped to any one of the facilities.

6. The method of managing surgical instruments according to claim 5, further comprising:
generating surgical instrument information indicating surgical instruments that are available to be selected for shipping to any one of the facilities based on the status information; and
providing the surgical instrument information along with the demand information.

7. The method of managing surgical instruments according to claim 5, wherein
the demand information comprises surgery schedule information regarding scheduled surgeries using the surgical robot systems at the facilities.

8. The method of managing surgical instruments according to claim 7, wherein
the surgery schedule information comprises information regarding a scheduled surgery date on which a surgery using one of the surgical robot systems is performed.

9. The method of managing surgical instruments according to claim 8, wherein
the providing the demand information comprises providing the demand information at a time according to the scheduled surgery date.

10. The method of managing surgical instruments according to claim 7, wherein
the surgery schedule information comprises at least one of content information regarding a content of a surgery and surgeon information regarding a surgeon operating one of the surgical robot systems.

11. The method of managing surgical instruments according to claim 10, wherein
the providing the demand information comprises generating the demand information based on at least one of the content information and the surgeon information, and information regarding a combination of the surgical instruments that need to be shipped to the one of the facilities.

12. The method of managing surgical instruments according to claim 1, wherein
the status information is associated with time period information indicating a period of time until each of the surgical instruments becomes available to be selected for shipping to any one of the facilities by the maintenance,
the method further comprises providing, based on the time period information, timing information indicating when one of the surgical instruments will be available to be selected for shipping to one of the facilities.

13. The method of managing surgical instruments according to claim 1, wherein
the maintenance comprises inspection of each of the surgical instruments.

14. The method of managing surgical instruments according to claim 13, wherein
the inspection comprises an inspection that determines if each of the surgical instrument is reusable.

15. The method of managing surgical instruments according to claim 1, further comprising obtaining usage information regarding a cumulative degree of use of each of the surgical instruments, wherein
the maintenance comprises inspection of each of the surgical instruments based on the usage information.

16. The method of managing surgical instruments according to claim 15, wherein
the usage information relates to at least one of cumulative number of times of use of each of the surgical instruments and a cumulative duration of use of each of the surgical instruments.

17. The method of managing surgical instruments according to claim 1, further comprising updating the status information for a surgical instrument designated for disposal based on disposal condition to the status information indicating that the surgical instrument is not selectable for shipping to the facilities.

18. The method of managing surgical instruments according to claim 5, wherein
the demand information is generated based on actual utilization performance information regarding an actual utilization performance of the surgical instruments at one of the facilities.

19. The method of managing surgical instruments according to claim 1, further comprising
outputting types and quantities of the surgical instruments that are selectable for shipping to any one of the facilities based on the status information.

20. The method of managing surgical instruments according to claim 1, further comprising
storing, for each of the surgical instruments, location information regarding a current location of each of the surgical instruments in association with the identification information of each of the surgical instruments.

21. An information management system that manages surgical instruments that are used in surgical robotic systems installed in each of facilities, that are collected, for each of which a maintenance comprising at least cleaning and sterilization is performed, and that are shipped to any one of the facilities, comprising:
a memory that stores, for each of the surgical instruments, status information regarding status of the surgical instruments in association with identification information for each of the surgical instruments; and
a controller that updates the status information corresponding to a surgical instrument that has been made available for shipping by the maintenance to the status information indicating that surgical instrument is available to be selected for shipping to any one of the facilities.
